(19) United States
(12) Patent Application Publication
(10) Pub. No.: US 2020/0009135 A1
(43) Pub. Date: Jan. 9, 2020

(54) THERAPEUTIC APPLICATIONS OF MALT1 INHIBITORS

(71) Applicant: MEDIVIR AB, Huddinge (SE)

(72) Inventors: Mark ALBERTELLA, Huddinge (SE); Fredrik ÖBERG, Huddinge (SE)

(21) Appl. No.: 16/482,399

(22) PCT Filed: Jan. 30, 2018
(86) PCT No.: PCT/EP2018/052286

§ 371 Date: Jul. 31, 2019

(30) Foreign Application Priority Data
Feb. 1, 2017 (SE) 1750082-8
May 24, 2017 (SE) 1750652-8

(51) Int. Cl. A61K 31/50; A61P 35/00

(52) U.S. Cl. CPC A61K 31/50; A61P 35/00

(57) ABSTRACT

The present invention relates to novel applications for inhibitors, notably small molecule inhibitors, of the protease in which the inhibitors are used in an immunooncology setting to treat certain cancers. This in turn means that the compounds are directed to immune components and not to the tumour tissue directly.

### FIELD OF THE INVENTION

The present invention relates to novel applications for inhibitors of the protease MALT1 within the field of cancer therapy in mammals, including humans.

### BACKGROUND OF THE INVENTION

MALT1 (mucosa associated lymphoid tissue lymphoma translocation protein 1) is an intracellular signalling protein, known from innate (natural killer cells NK, dendritic cells DC, and mast cells) and adaptive immune cells (T cells and B cells). The function of MALT1 is best known in the context of T cell receptor (TCR signalling), where it mediates nuclear factor κB (NFκB) signalling leading to T cell activation and proliferation. Accordingly, MALT1 was of interest in the mechanism of autoimmune and inflammatory pathologies. Additionally, it was noted that constitutive (dysregulated) MALT1 activity is associated with MALT lymphoma and activated B cell-like diffuse large B Cell lymphoma (ABC-DLBCL).

MALT1 is a paracaspase with both scaffold functions (contributing to the assembly of other signalling complexes) and protease functions cleaving a limited repertoire of proteins. The MALT1 proteolytic activity appears essential for T cell activation and also the B cell lymphomas identified above.

Several groups have identified inhibitors of MALT1 activity as potential therapeutics. Rebaud et al Nat Immunol 2008 9(3), 272-81 describes a warhead-equipped substrate analogue zVRPRfmk, while Lim et al J Med Chem 2015 58(21) 8591-8502 describes the small molecule MALT1 inhibitor MI2. Nagel et al Cancer Cell 2012 22(6) 825-37 describes another small molecule inhibitor mepazine. Characteristic for these prior art inhibitors of MALT1, is that the compounds are proposed for autoimmune or inflammatory pathways, or cancers dependent on dysregulated NFκB pathway activity.

Similarly, Novartis WO2015/181747 discloses a genus of small molecule inhibitors of MALT1 with the formula (I)

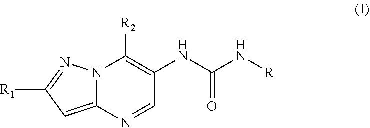

in which R₁, R₂ and R are defined below. Novartis assay their compounds in a MALT1 biochemical assay, and also an NFκB reporter gene assay driven by ectopic expression of the cIAP2-MALT1 fusion protein typical of MALT-lymphomas, and an IL2 promoter-driven reporter gene assay. Representative disorders treatable with the Novartis compounds are proposed to be autoimmune disorders and inflammatory diseases such as rheumatoid arthritis. No oncology data is provided, but ABC-DLBCL, eg with activating mutations in card 11, and MALT lymphoma are highlighted as potential indications. These cancers are well known to be dependent on dysregulated NFκB pathway activity. It is also striking that the list of adjunct therapies provided by Novartis for use in combination with their compounds is dominated by immunosuppressive agents, such as cyclosporine, rapamycin, methotrexate and the like, which would be expected to impede any immuno-oncologic activities in the affected tissue.

Novartis have further published, in WO2017/081641, a subset of the compounds in the paragraph immediately above, also predominantly intended for autoimmune and inflammatory disorders mediated by MALT1. WO2017/081641 does, at page 79, speculate on using the compounds in oncological disorders, but specifically defines that it is oncological disorders that are characterised by dysregulated NF-kB regulation.

### SUMMARY OF THE INVENTION

MALT1 inhibitors have previously been proposed for treatment of cancers in which the NFκB pathway is overactive (e.g. ABC-DLBCL). Blockade/inhibition of MALT1 directly down-regulates the NFκB pathway in such cancers, resulting in treatment. The present invention is based on the appreciation of an additional activity of MALT1 inhibitors, which is independent of the direct inhibition of dysregulated NFκB pathway activity in tumour cells. Rather it is a function of the effect on various components of the immune system of inhibiting MALT1.

In other words, rather than, or in addition to, MALT1 inhibitors acting directly on the tumour tissue, with all the difficulties of reaching the target organ which this implies, the present invention envisages that the site of MALT1 action is within specified T cell populations of a subject. This appreciation dramatically expands the range of cancers for which administration of a MALT1 inhibitor is desirable, because a MALT1 inhibitor can now be used as an immunomodulatory agent to activate or augment the T cell anti-cancer response in a subject, irrespective of whether the cancer has dysregulated NFκB pathway activity. As shown in Biology Example 6 below and contrary to the speculation in the above Novartis publications, many, if not most, cancer tissues are essentially unaffected by exposure to the small molecule MALT1 inhibitors exemplified in those publications.

The invention thus provides a MALT1 inhibitor for use as an immunomodulatory agent in the prevention or treatment of cancer, independently of dysregulated NFκB pathway activation within the cancer cells. In other words, the present invention provides a method for the prevention or treatment of cancer in a subject, the method comprising administering to said subject a MALT 1 inhibitor as an immunomodulatory agent. The method of the invention may additionally comprise administering to the subject a further therapeutic agent. The further therapeutic agent may be:
(i) an additional immunomodulatory agent which blocks or inhibits an immune system checkpoint, which checkpoint may or may not be a component of the NFκB pathway; and/or
(ii) an agent which directly stimulates an immune effector response, such as a cytokine or chemokine (or an agent which stimulates production of either), a tumour specific adoptively transferred T cell population, or an antibody specific for a protein expressed by a tumour cell; and/or
(iii) a composition comprising a tumour antigen or immunogenic fragment thereof; and/or
(iv) a chemotherapeutic agent.

The invention also provides per se novel MALT1 inhibitors, such as (S)-1-(6-(4-(aminomethyl)-1H-pyrazol-1-yl)-5-chloropyridin-3-yl)-3-(2-chloro-7-(1-methoxyethyl)pyrazolo[1,5-a]pyrimidin-6-yl)urea (1f). These MALT1 inhibitors are suitable for use in the methods of the invention.

### BRIEF DESCRIPTION OF THE FIGURES

FIG. 1 depicts the ratio of FOXP3+CD25+ as a percentage of control for three concentrations of a MALT1 inhibitor, in three donors, as described more fully in Biological Example 2;

FIG. 2 depicts T_{reg} suppressive activity as the percentage suppression of CD4+ cell proliferation for various T_{eff}:T_{reg} ratios exposed to three concentrations of a MALT1 inhibitor in three donors, as described more fully in Biological Example 2;

FIG. 3 depicts ex vivo effect of the compound of Example 2 on percentage IFNg+CD8+NLV+ T-cells in a human blood loop system. Each blood loop contained 2 mL of freshly taken human whole blood from HLA-A2+ and CMV+ donors. Compound (Example 2) 4 uM final concentration and/or CMV lysate final concentration 1 ug/mL were added directly after blood sampling and loops were set to rotate at 37° C. After 2 hours, Brefeldin A was added to inhibit secretion of cytokines, allowing intracellular analysis of cytokines after 6 hour incubation, as further described in Biological Example 3;

FIG. 4 depicts in vivo effect on T_{reg} and T_{eff} cells of Example 2 in the MB49 mouse bladder cancer model. Percent of T_{reg} cells (FOXP+CD3+CD4+) and T_{eff} cells (CD25+CD8+CD3+) infiltrating the tumour (T) and in tumour-draining lymph nodes (TDLN).

### DETAILED DESCRIPTION OF THE INVENTION

### General

It is to be understood that different applications of the disclosed products and methods may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural referents unless the content clearly dictates otherwise. Thus, for example, reference to “an inhibitor” includes two or more such inhibitors, or reference to “an oligonucleotide” includes two or more such oligonucleotide and the like.

A “subject” as used herein refers to an animal, typically a mammal. For example, subject may refer to, for example, primates (e.g., humans, male or female), cows, sheep, goats, horses, dogs, cats, rabbits, rats, mice, fish, birds and the like, in certain embodiments, the subject is a primate. The subject is preferably a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof), in another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” refers to an amount of a substance that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of a MALT1 inhibitor that, when administered to a subject, is sufficient to achieve an immunomodulatory effect which at least partially alleviates, inhibits, prevents and/or ameliorates a cancerous condition, independently of dysregulated NFkB pathway activation within the cancer cells.

All publications, patents and patent applications cited herein, whether supra or infra, are hereby incorporated by reference in their entirety.

### Immune System Involvement in Cancer

Accumulating evidence shows a correlation between tumor-infiltrating lymphocytes in cancer tissue and favorable prognosis in various malignancies. In particular, the presence of CD8+ T-cells and a high ratio of CD8+ effector T-cells compared to FoxP3+ regulatory T-cells (T_{regs}) correlates with improved prognosis and long-term survival in solid cancers, e.g. colorectal-, and ovarian cancer, hepatocellular carcinoma; bladder cancer, malignant melanoma; and renal cell carcinoma. Similarly, high levels of infiltrating T_{regs} have been found to be associated with poor prognosis in a number of cancers, e.g. ovarian carcinoma, breast cancer, cervical and renal carcinoma, and malignant melanoma. Therapies resulting in reduction of T_{regs} and thereby changing the T_{eff}/T_{reg} ratio would therefore be expected to have a positive influence on cancer outcome.

FoxP3 (forkhead box P3), also known as scurfin, is a protein involved in immune system responses and appears to function as a master regulator of the regulatory pathway in the development and function of regulatory T cells. While the precise control mechanism has not yet been established, FOX proteins belong to the forkhead/winged-helix family of transcriptional regulators and are presumed to exert control via similar DNA binding interactions during transcription. In regulatory T cell model systems, the FOXP3 transcription factor occupies the promoters for genes involved in regulatory T-cell function.

FoxP3 is a specific marker for natural T regulatory cells (nT_{regs}, a lineage of T cells) and adaptive/induced T regulatory cells (a/iT_{regs}), also identified by other less specific markers such as CD25 or CD45RB. In animal studies, T_{regs} that express FOXP3 are critical in the transfer of immune tolerance, especially self-tolerance. The induction or administration of FoxP3 positive T cells has, in animal studies, led to marked reductions in autoimmune disease severity in models of diabetes, MS, asthma, inflammatory bowel disease and renal disease. Human trials using regulatory T cells to treat graft versus host disease have shown efficacy.

Several FOXP3 recognising antibodies are commercially available, and immunohistochemistry (IHC) or flow cytometry methods are widely available for recognising Fox P3 positive T_{reg} lymphocytes, and the tumours which they infiltrate.

CD8+T effector lymphocytes, also known as cytotoxic T lymphocyte or CTL bearing the CD8 glycoprotein, which binds to the constant portion of the class 1 MHC molecule during antigen recognition and apoptosis. CD8+T effector lymphocytes are readily identified by IHCor by flow cytometry.

CD4+T effector lymphocytes, also known as T helper cells, express the surface protein CD4, a coreceptor of the TCR complex which binds to a different location on the class II MHC molecule.

### Methods for the Prevention or Treatment of Cancer

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

The methods of the invention concern preventing or treating cancer. The cancer is preferably of a type which is not characterised by abnormally high activity in the NF-κB pathway.

The cancer may be characterised by the presence of both infiltrating regulatory T cells (T_{reg} cells) and infiltrating effector T cells (T_{eff} cells) in the tumour. T_{reg} cells are typically characterised as FOXP3+. T_{eff} cells are typically characterised as CD4+ or CD8+. The number of T_{reg} and T_{eff} cells in a tumour may be determined by any suitable method, but typically this involves the quantification of each cell type in a tumour sample or a sample from a tumour draining lymph node. Suitable methods for the quantification of cells include flow cytometry, which may be performed in accordance with the protocols set out in the Examples.

The cancer may be prostate cancer, brain cancer, breast cancer, colorectal cancer, pancreatic cancer, ovarian cancer, lung cancer, cervical cancer, liver cancer, head/neck/throat cancer, skin cancer, bladder cancer or a hematologic cancer. The cancer may take the form of a tumour or a blood born cancer. The tumour may be solid. The tumour is typically malignant and may be metastatic. The tumour may be an adenoma, an adenocarcinoma, a blastoma, a carcinoma, a desmoid tumour, a desmopolastic small round cell tumour, an endocrine tumour, a germ cell tumour, a lymphoma, a leukaemia, a sarcoma, a Wilms tumour, a lung tumour, a colon tumour, a lymph tumour, a breast tumour or a melanoma.

Types of blastoma include hepatblastoma, glioblastoma, neuroblastoma or retinoblastoma. Types of carcinoma include colorectal carcinoma or heptacellular carcinoma, pancreatic, prostate, gastric, esophegal, cervical, and head and neck carcinomas, and adenocarcinoma. Types of sarcoma include Ewing sarcoma, osteosarcoma, rhabdomyosarcoma, or any other soft tissue sarcoma. Types of melanoma include Lentigo maligna, Lentigo maligna melanoma, Superficial spreading melanoma, Acral lentiginous melanoma, Mucosal melanoma, Nodular melanoma, Polypoid melanoma, Desmoplastic melanoma, Amelanotic melanoma, Soft-tissue melanoma, Melanoma with small nevus-like cells, Melanoma with features of a Spitz nevus and Uveal melanoma. Types of lymphoma and leukaemia include Precursor T-cell leukemia/lymphoma, acute myeloid leukaemia, chronic myeloid leukaemia, acute lymphcytic leukaemia, Follicular lymphoma, Diffuse large B cell lymphoma, Mantle cell lymphoma, chronic lymphocytic leukemia/lymphoma, MALT lymphoma, Burkitt's lymphoma, Mycosis fungoides, Peripheral T-cell lymphoma, Nodular sclerosis form of Hodgkin lymphoma, Mixed-cellularity subtype of Hodgkin lymphoma. Types of lung tumour include tumours of non-small-cell lung cancer (adenocarcinoma, squamous-cell carcinoma and large-cell carcinoma) and small-cell lung carcinoma.

The cancer may preferably be selected from
bladder cancer,
colon cancer,
hepatocellular cancer, or
Small Cell or Non-Small Cell lung cancer.

### Combinations

The method of the invention may additionally comprise administering to the subject a further therapeutic agent. The further therapeutic agent may preferably be:
(v) an additional immunomodulatory agent which blocks or inhibits an immune system checkpoint, which checkpoint may or may not be a component of the NFκB pathway; and/or
(vi) an agent which directly stimulates an immune effector response, such as a cytokine or chemokine (or an agent which stimulates production of either), a tumour specific adoptively transferred T cell population, or an antibody specific for a protein expressed by a tumour cell; and/or
(vii) a composition comprising a tumour antigen or immunogenic fragment thereof; and/or
(viii) a chemotherapeutic agent.

The MALT1 inhibitor may be administered either simultaneously with, or before or after, the further therapeutic agent. The MALT1 inhibitor may be administered separately, by the same or different route of administration, or together in the same pharmaceutical composition as the further therapeutic agent.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula (I) and a co-agent, are both administered to a patient simultaneously in the form of a single entity or dosage. The term “non-fixed combination” means that the active ingredients, e.g. a compound of formula (I) and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients. In one embodiment, the Invention provides a product comprising a MALT1 inhibitor, such as a compound of formula (I) and at least one other therapeutic agent as a combined preparation for simultaneous, separate or sequential use in therapy. Products provided as a combined preparation include a composition comprising the MALT1 inhibitor such as a compound of formula (I) and the other therapeutic agent(s) together in the same pharmaceutical composition, or the compound of formula (I) and the other therapeutic agent(s) in separate form, e.g. in the form of a kit. In one embodiment, the invention provides a pharmaceutical composition for use in therapy comprising a compound of formula (I) and an additional immunomodulatory agent or a composition comprising a tumour antigen or immunogenic fragment thereof. Optionally, the pharmaceutical composition may comprise a pharmaceutically acceptable excipient.

It will be appreciated that many, of the further therapeutic agents used in the methods of the invention may be biologicals requiring intravenous, intraperitoneal or depot administration. In a favoured embodiment of the invention, the MALT1 inhibitor is an orally administered small molecule inhibitor and the further therapeutic agent is administered parenterally, for example intravenously, intraperitoneally or as a depot.

### Immune System Checkpoint

Effector T cell activation is normally triggered by the T cell receptor recognising antigenic peptide presented by the MHC complex. The type and level of activation achieved is then determined by the balance between signals which stimulate and signals which inhibit the effector T cell response. The term “immune system checkpoint” is used herein to refer to any molecular interaction which alters the balance in favour of inhibition of the effector T cell response. That is, a molecular interaction which, when it occurs, negatively regulates the activation of an effector T cell. Such an interaction might be direct, such as the interaction between a ligand and a cell surface receptor which transmits an inhibitory signal into an effector T cell. Or it might be indirect, such as the blocking or inhibition of an interaction between a ligand and a cell surface receptor which would otherwise transmit an activatory signal into the effector T cell, or an interaction which promotes the upregulation of an inhibitory molecule or cell, or the depletion by an enzyme of a metabolite required by the effector T cell, or any combination thereof.

Examples of immune system checkpoints include:
a) The interaction between Indoleamine 2,3-dioxygenase (IDO1) and its substrate;
b) The interaction between PD1 and PDL1 and/or PD1 and PDL2;
c) The interaction between CTLA4 and CD86 and/or CTLA4 and CD80;
d) The interaction between B7-H3 and/or B7-H4 and their respective ligands;
e) The interaction between HVEM and BTLA;
f) The interaction between GAL9 and TIM3;
g) The interaction between MHC class I or II and LAG3; and
h) The interaction between MHC class I or II and KIR
i) The interaction between OX40(CD134) and OX40L (CD252)
k) The interaction between CD40 and CD40L (CD154)
l) The interaction between 4-1 BB (CD137) and ligands including 4-1 BBL
m) The interaction between GITR and ligands including GITRL

A preferred checkpoint for the purposes of the present invention is checkpoint (b), namely the interaction between PD1 and either of its ligands PD-L1 and PD-L2. PD1 is expressed on effector T cells. Engagement with either ligand results in a signal which downregulates activation. The ligands are expressed by some tumours. PD-L1 in particular is expressed by many solid tumours, including melanoma. These tumours may therefore down regulate immune mediated anti-tumour effects through activation of the inhibitory PD-1 receptors on T cells. By blocking the interaction between PD1 and one or both of its ligands, a checkpoint of the immune response may be removed, leading to augmented anti-tumour T cell responses. Therefore PD1 and its ligands are examples of components of an immune system checkpoint which may preferably be targeted in the method of the invention

Another preferred checkpoint for the purposes of the present invention is checkpoint (c), namely the interaction between the T cell receptor CTLA-4 and its ligands, the B7 proteins (B7-1 and B7-2). CTLA-4 is ordinarily upregulated on the T cell surface following initial activation, and ligand binding results in a signal which inhibits further/continued activation. CTLA-4 competes for binding to the B7 proteins with the receptor CD28, which is also expressed on the T cell surface but which upregulates activation. Thus, by blocking the CTLA-4 interaction with the B7 proteins, but not the CD28 interaction with the B7 proteins, one of the normal check points of the immune response may be removed, leading to augmented anti-tumour T cell responses. Therefore CTLA4 and its ligands are examples of components of an immune system checkpoint which may preferably be targeted in the method of the invention

### Immunomodulatory Agent

An “immunomodulatory agent” is used herein to mean any agent which, when administered to a subject, blocks or inhibits the action of an immune system checkpoint, resulting in the upregulation of an immune effector response in the subject, typically a T cell effector response, which preferably comprises an anti-tumour T cell effector response.

The immunomodulatory agent used in the method of the present invention may block or inhibit any of the immune system checkpoints described above. The agent may be an antibody or any other suitable agent which results in said blocking or inhibition. The agent may thus be referred to generally as an inhibitor of a said checkpoint.

An “antibody” as used herein includes whole antibodies and any antigen binding fragment (i.e., “antigen-binding portion”) or single chains thereof. An antibody may be a polyclonal antibody or a monoclonal antibody and may be produced by any suitable method. Examples of binding fragments encompassed within the term “antigen-binding portion” of an antibody include a Fab fragment, a F(ab′)2 fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody.

Preferred antibodies which block or inhibit the CTLA-4 interaction with B7 proteins include ipilumumab, tremelimumab, or any of the antibodies disclosed in WO2014/207063. Other molecules include polypeptides, or soluble mutant CD86 polypeptides. Ipilumumab is most preferred.

Preferred antibodies which block or inhibit the PD1 interaction with PD-L1 include Nivolumab, Pembrolizumab, Lambrolizumab, Pidilzumab, BGB-A317 and AMP-224. Nivolumab or pembrolizumab is most preferred. Anti-PD-L1 antibodies include atezolizemab, avelumab or durvalumab, MEDI-4736 and MPDL3280A.

Preferred antibodies which block or inhibit the interaction between 4-1 BB and its ligand include utomilumab.

Other suitable inhibitors include small molecule inhibitors (SMI), which are typically small organic molecules. Preferred inhibitors of IDO1 include Epacadostat (INCB24360), Indoximod, GDC-0919 (NLG919) and F001287. Other inhibitors of IDO1 include 1-methyltryptophan (1MT).

### Direct Stimulation of Immune Effector Responses

As used herein, “an agent which directly stimulates an immune effector response” means any suitable agent, but typically refers to a cytokine or chemokine (or an agent which stimulates production of either), a tumour specific adoptively transferred T cell population, or an antibody specific for a protein expressed by a tumour cell.

The cytokine may be an interferon selected from IFNα, IFNβ, IFNγ and IFNλ, or an interleukin, preferably IL-2. The chemokine may be an inflammatory mediator, for example selected from CXCL9, 10, and 11, which attract T cells expressing CXCR3. The agent which stimulates production of a cytokine or chemokine may be an adjuvant suitable for administration to humans. A preferred example is Bacille Calmette-Guerin (BCG), which is typically administered intravesically (i.e. urethral catheter) for treatment of bladder cancer. A typical dosage regime of BCG for bladder cancer is once per week for six weeks, but given its long safety history it is also administered indefinitely as maintenance. BCG has been shown to stimulate immune responses to bladder cancer. BCG has also been used as an adjuvant in combination with compositions which comprise tumour antigens (i.e. with cancer vaccines), particularly for colon cancer when it is administered typically intradermally. Such uses of BCG are also envisaged in the present invention.

The tumour specific adoptively transferred T cell population directly increases the size of the tumour specific T cell population in an individual, and may be generated by any suitable means. However, typically the process involves isolating tumour specific T cells from a tumour sample taken from a patient, and selectively culturing those cells before returning the expanded population of tumour-specific T cells to the patient. Alternatively a tumour specific T cell population may be produced by genetic engineering of the T cell receptor locus, followed by expansion of the altered cell.

Antibodies specific for proteins expressed by a tumour cell typically stimulate immune activity by binding to the tumour cell and promoting destruction of the cell via antibody-dependent cell-mediated cytotoxicity (ADCC). Preferred examples of antibodies of this type include anti-CD20 antibodies such as ofatumumab or rituximab, and anti-CD52 antibodies such as alemtuzumab.

### Compositions Comprising Tumour Antigens

A composition as used in the method of the invention may comprise any tumour antigen or any antigenic fragment thereof. Such a composition may alternatively be described as a vaccine against the said tumour antigen, which stimulates an adaptive immune response to the antigen when administered to a subject. The tumour antigen or fragment may be present in the composition in polypeptide (or peptide) form, or may be encoded by a nucleic acid, for example an RNA or DNA molecule, or may be present as whole cells (e.g. an autologous tumour cell vaccine).

Tumour antigens are typically molecules which are located on the surface of the tumour cell. Tumour antigens may e selected from proteins which are overexpressed in tumour cells compared to a normal, non-cancerous cell. Tumour antigens include antigens expressed in cells which are not cancerous but are associated with a tumour. Antigens which are connected with tumour-supplying vessels or formation thereof, in particular those antigens which are associated with neo-vascularization, e.g. VEGF, bFGF, are also included herein. Antigens associated with a tumour furthermore include antigens from cells or tissues, typically embedding the tumour.

Tumour antigens can be divided further into tumour-specific antigens (TSAs) and tumour-associated-antigens (TAAs). TSAs can only be expressed by tumour cells and not by normal “healthy” cells. They typically result from a tumour specific mutation. TAAs, which are more common, may be expressed by both tumour and healthy cells. These antigens are recognized and the antigen-expressing cell can be destroyed by cytotoxic T cells. Additionally, tumour antigens can also occur on the surface of the tumour in the form of, e.g., a mutated receptor. In this case, they can be recognized by antibodies. Further, tumour associated antigens may be classified as tissue-specific antigens, examples of which include melanocyte-specific antigens, cancer-testis antigens and tumour-specific antigens. Cancer-testis antigens are typically understood to be peptides or proteins of germ-line associated genes which may be activated in a wide variety of tumours. Human cancer-testis antigens may be further subdivided into antigens which are encoded on the X chromosome, so-called CT-X antigens, and those antigens which are not encoded on the X chromosome, the so-called non-X CT antigens. Cancer-testis antigens which are encoded on the X-chromosome comprise, for example, the family of melanoma antigen genes, the so-called MAGE-family. The genes of the MAGE-family may be characterised by a shared MAGE homology domain (MHD). Each of these antigens, i.e. melanocyte-specific antigens, cancer-testis antigens and tumour-specific antigens, may elicit autologous cellular and humoral immune responses. Preferred tumour antigens of the invention include a melanocyte-specific antigen, a cancer-testis antigen or a tumour-specific antigen, preferably a CT-X antigen, a non-X CT-antigen, a binding partner for a CT-X antigen or a binding partner for a non-X CT-antigen or a tumour-specific antigen, more preferably a CT-X antigen, a binding partner for a non-X CT-antigen or a tumour-specific antigen.

Particularly preferred tumour antigens are selected from 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, calreticulin, CAMEL, CASP-8/m, cathepsin B, cathepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R171, HLA-A11/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1R, IL-13Ra2, IL-2R, IL-5, immature laminin receptor, kallikrein-2, kallikrein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MC1R, M-CSF, ME1/m, mesothelin, MG50/PXDN, MMP11, MN/CA IX-antigen, MRP-3, MUC-1, MUC-2, MUM-1/m, MUM-2/m, MUM-3/m, myosin class I/m, NA88-A, N-acetylglucosaminyltransferase-V, Neo-PAP, Neo-PAP/m, NFYC/m, NGEP, NMP22, NPM/ALK, N-Ras/m, NSE, NY-ESO-B, NY-ESO-1, OA1, OFA-iLRP, OGT, OGT/m, OS-9, OS-9/m, osteocalcin, osteopontin, p15, p190 minor bcr-abl, p53, p53/m, PAGE-4, PAI-1, PAI-2, PAP, PART-1, PATE, PDEF, Pim-1-Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD168, RU1, RU2, S-100, SAGE, SART-1, SART-2, SART-3, SCC, SIRT2/m, Sp17, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGFbeta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, and WT1.

Most preferred tumour antigens are selected from p53, CAI25, EGFR, Her2/neu, hTERT, PAP, MAGE-A1, MAGE-A3, Mesothelin, MUC-1, GP100, MART-1, Tyrosinase, PSA, PSCA, PSMA, STEAP-1, VEGF, VEGFR1, VEGFR2, Ras, CEA or WT1.

Tumour antigens also may encompass idiotypic antigens associated with a cancer or tumour disease, particularly lymphoma or a lymphoma associated disease, wherein said idiotypic antigen is an immunoglobulin idiotype of a lymphoid blood cell or a T cell receptor idiotype of a lymphoid blood cell.

### Chemotherapeutic Agents

As used herein, “chemotherapeutic agent” means any agent which has been approved for use as a chemotherapy for cancer. Examples include but are not limited to: all-trans retinoic acid, actimide, azacitidine, azathioprine, bleomycin, carboplatin, capecitabine, cisplatin, chlorambucil, cyclophosphamide, cytarabine, daunorubicin, docetaxel, doxifluridine, doxorubicin, epirubicin, etoposide, fludarabine, fluorouracil, gemcitabine, hydroxyurea, idarubicin, irinotecan, lenalidomide, leucovorin, mechlorethamine, melphalan, mercaptopurine, methotrexate, mitomycin, mitoxantrone, oxaliplatin, paclitaxel, pemetrexed, revlimid, temozolomide, teniposide, thioguanine, thiotepa, valrubicin, vinblastine, vincristine, vindesine and vinorelbine. a chemotherapeutic agent for use in the combinations described herein may, itself, be a combination of different chemotherapeutic agents. suitable combinations include a combination of 5-fluorouracil (5-FU), leucovorin, and oxaliplatin (may be referred to as FOLFOX), or a combination of irinotecan, 5-FU, and leucovorin (may be referred to as IFL).

In a particular embodiment, provided herein is a method for the treatment of bladder cancer comprising the administration of a MALT1 inhibitor and at least one of BCG and a chemotherapeutic agent selected from mitomycin, valrubicin, docataxel, thiotepa and gemcitabine, wherein at least the BCG and the chemotherapeutic agent are preferably administered intravesically, i.e. via urethral catheter.

In another embodiment, provided herein is a method for the treatment of colon cancer comprising the administration of a MALT1 inhibitor and at least one of BCG and a composition comprising a tumour antigen, preferably an autologous tumor cell vaccine. At least the BCG and the composition comprising a tumour antigen are preferably administered parenterally, optionally as a single combined preparation.

### MALT 1 Inhibitors

The MALT1 inhibitor of the invention will typically have good ability to get into T cells, for example an RNA-based drug or preferably a small molecule drug. In a favoured embodiment the MALT1 inhibitor is a small molecule drug which is orally administered. In certain forms of cancer it may be preferable to administer the MALT1 inhibitor locally, eg topically, or intravesically in the case of bladder cancer. Where the subject receives other medicaments, whether as part of a method of the invention or otherwise, it may be convenient to administer the MALT1 inhibitor by the same route as the other medicaments. Such routes may include parenterally in the case of many immunomodulatory agents, or as TACE for hepatocellular cancer or intrathecally/intracerebrally for glioblastoma, astrocytoma or other nerve tissue cancers.

The MALT1 inhibitor may change the ratio of T_{reg}/T_{eff} cells infiltrating a tumour in favour of the T_{eff} cells. This may typically be achieved by reducing the number of infiltrating T_{reg} cells whilst maintaining or increasing the level of infiltrating T_{eff} cells. The ratio of T_{reg}/T_{eff} cells in a tumour may be determined by any suitable method, but typically involves the quantification of each cell type in a tumour sample or a sample from a tumour draining lymph node. Suitable methods include flow cytometry, which may be performed in accordance with the protocols set out in the Examples.

The MALT1 inhibitor of the invention is preferably a small molecule drug of the formula I

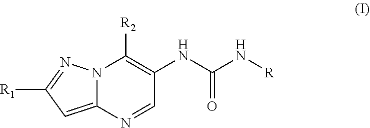

wherein,
R₁ is halogen, cyano, or C₁-C₃ alkyl optionally substituted by halogen;
R₂ is C₁-C₆ alkyl optionally substituted one or more times by C₁-C₆ alkyl, C₂-C₆ alkenyl, hydroxyl, N,N-di-C₁-C₆ alkylamino, N-mono-C₁-C₆ alkylamino, O—Rg, Rg, phenyl, or by C₁-C₆ alkoxy wherein said alkoxy again may optionally be substituted by C₁-C₆ alkoxy, N,N-di-C₁-C₆ alkylamino, Rg or phenyl; C₃-C₆ cycloalkyl optionally substituted by C₁-C₆ alkyl, N,N-di-C₁-C₆ alkylamino or C₁-C₆ alkoxy-C₁-C₆ alkyl, and/or two of said optional substituents together with the atoms to which they are bound may form an annulated or spirocyclic 4-6 membered saturated heterocyclic ring comprising 1-2 O atoms; phenyl optionally substituted by C₁-C₆ alkoxy; a 5-6 membered heteroaryl ring having 1 to 3 heteroatoms selected from N and O said ring being optionally substituted by C₁-C₆ alkyl which may be optionally substituted by amino or hydroxy; Rg; or N,N-di-C₁-C₆alkyl amino carbonyl; wherein
Rg is a 5-6 membered heterocyclic ring having 1-3 heteroatoms selected from N and O said ring being optionally substituted by C₁-C₆ alkyl, C₁-C₆ alkoxy-C₁-C₆ alkyl, C₁-C₆ alkoxy-carbonyl; R is phenyl independently substituted two or more times by Ra, 2-pyridyl independently substituted one or more times by Rb, 3-pyridyl independently substituted one or more times by Rc, or 4-pyridyl independently substituted one or more times by Rd; wherein Ra independently from each other is halogen; cyano; —COOC₁-C₆alkyl; C₁-C₆ alkoxy; C₁-C₆ alkyl optionally substituted by halogen or a 5-6 membered heterocyclyl ring having 1 to 2 heteroatoms selected from N and O which ring is optionally substituted by C₁-C₆ alkyl; a 5-6 membered heteroaryl ring having 1 to 3 heteroatoms selected from N and O said ring being optionally substituted by amino, C₁-C₆ alkyl optionally substituted by amino or hydroxy, or by N-mono- or N,N-di-C₁-C₆ alkylamino carbonyl; and/or
two Ra together with the ring atoms to which they are bound may form a 5 to 6 membered heterocyclic or heteroaromatic ring having 1 to 2 N atoms, any such ring being optionally substituted by C₁-C₆ alkyl or oxo;
Rb, Rc and Rd independently from each other are halogen; oxo; hydroxy; cyano; C₁-C₆ alkoxy optionally substituted by halogen; C₁-C₆ alkoxy carbonyl; phenyl; N,N-di-C₁-C₆ alkyl amino; C₁-C₆ alkyl optionally substituted by halogen or phenyl; a 5-6 membered heteroaryl ring having 1 to 3 N atoms said ring being optionally substituted by C₁-C₆ alkyl optionally substituted by amino or hydroxy, or by mono- or di-N—C₁-C₆ alkylamino carbonyl; O—Rh; or Rh; wherein
Rh is a 5-6 membered heterocyclyl ring having 1 to 4 heteroatoms selected from N, O and S said ring being optionally substituted by C₁-C₆ alkyl, hydroxyl or oxo.

In a particular embodiment, the MALT1 inhibitor is of formula (I), wherein R, is halogen; R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkyl amino, O—Rg, Rg, phenyl, or by C₁-C₆ alkoxy, wherein said alkoxy again may optionally be substituted by C₁-C₆ alkoxy, N,N-di-C₁-C₆ alkylamino, Rg or phenyl; wherein

Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is 2-pyridyl independently substituted one or more times by Rb, 3-pyridyl independently substituted one or more times by Rc, or 4-pyridyi independently substituted one or more times by Rd; and
Rb, Rc and Rd are as defined in Embodiment A;
or a pharmaceutically acceptable salt thereof.

In a particular embodiment, the MALT1 inhibitor is of formula (I), wherein R, is cyano;

R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkyl amino, O—Rg, Rg, phenyl, or by C₁-C₆ alkoxy, wherein said alkoxy again may optionally be substituted by C₁-C₆ alkoxy, N,N-di-C₁-C₆ alkylamino, Rg or phenyl; wherein
Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is 2-pyridyl independently substituted one or more times by Rb, 3-pyridyl independently substituted one or more times by Rc, or 4-pyridyi independently substituted one or more times by Rd; and
Rb, Rc and Rd are as defined in Embodiment A;
or a pharmaceutically acceptable salt thereof.

In a particular embodiment, the MALT1 inhibitor is of formula (I), wherein R, is C₁-C₃ alkyl, optionally substituted by halogen;

R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkyl amino, O—Rg, Rg, phenyl, or by C₁-C₆ alkoxy, wherein said alkoxy again may optionally be substituted by C₁-C₆ alkoxy, N,N-di-C₁-C₆ alkylamino, Rg or phenyl; wherein
Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is 2-pyridyl independently substituted one or more times by Rb, 3-pyridyl independently substituted one or more times by Rc, or 4-pyridyi independently substituted one or more times by Rd; and
Rb, Rc and Rd are as defined in Embodiment A;
or a pharmaceutically acceptable salt thereof.

In a particular embodiment, the MALT1 inhibitor is of formula (I), wherein R, is chloro, and the remaining substituents are as defined therein.

A favoured embodiment the present invention, denoted 2017-1, employs a MALT1 inhibitor of the formula (II) or a pharmaceutically acceptable salt thereof,

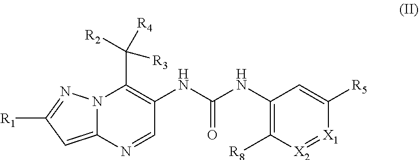

wherein
R₁ is fluoro, chloro, methyl or cyano;
R₂ and R₃ are independently from each other C₁-C₆ alkoxy optionally substituted by C₁-C₆ alkoxy; C₁-C₈, alkyl optionally substituted by halogen or C₁-C₆ alkoxy; amino optionally substituted by C₁-C₆ alkyl; phthalimido; or hydroxy optionally substituted by a 5 or 6 membered heterocyclic ring comprising a nitrogen or oxygen heteroatom wherein said ring is optionally substituted by C₁-C₃ alkyl carbonyl;
or R₂ and R₃ together with carbon atom to which they are attached form a 3-5 membered carbocyclic ring or heterocyclic ring comprising 1 heteroatom selected from N and O;
R₄ is hydrogen; C₁-C₆ alkyl optionally substituted by C₁-C₆ alkoxy;
X₁ is N, N—O or CR₆;
X₂ is N or CR₇;
R₅ is chloro; cyano; or C₁-C₆ alkyl optionally substituted by halogen and/or hydroxy;
R₆ is hydrogen; oxo; methoxy; 1,2,3-triazole-2-yl; or aminocarbonyl substituted at the nitrogen atom by R₉ and R₁₀;
R₇ is hydrogen; C₁-C₆ alkyl optionally substituted by halogen and/or hydroxy; or N, N-dimethylaminocarbonyl;
R₈ is hydrogen; C₁-C₆ alkoxy optionally substituted by methoxy or amino;
R₉ and R₁₀ are independently of each other hydrogen; C₁-C₆ alkyl optionally substituted by C₁-C₆ alkoxy, N-mono-C₁-C₆ alkyl amino, or N, N-di-C₁-C₆ alkyl amino; or
R₉ and R₁₀ together with the nitrogen atom to which they are attached form a 5-7 membered heterocyclic ring having one, two or three ring hetero atoms selected from the group consisting of oxygen, nitrogen and sulphur, that ring being optionally substituted by C₁-C₆ alkyl, hydroxy or oxo;
with the proviso that X₁ and X₂ must not be N at the same time, or X₁ must not be N—O when X₂ is N.

Embodiment 2017-2 employs a MALT1 inhibitor of embodiment 2017-1 or a pharmaceutically acceptable salt thereof, wherein

R₁ is fluoro or chloro;
R₂ is C₁-C₆ alkyl optionally substituted by C₁-C₆ alkoxy;
R₃ is C₁-C₆ alkoxy optionally be substituted by C₁-C₆ alkoxy;
R₄ is hydrogen;

### X₁ is N;

X₂ is CR₇;
R₅ is chloro; cyano; difluoromethyl; trifluoromethyl;
R₇ is hydrogen; and
R₈ is hydrogen.

Embodiment 2017-3 employs a MALT1 inhibitor of embodiment 2017-1 or a pharmaceutically acceptable salt thereof, wherein

R₁ is fluoro or chloro;
R₂ is C₁-C₆ alkyl optionally substituted by C₁-C₆ alkoxy;
R₃ is C₁-C₆ alkoxy optionally be substituted by C₁-C₆ alkoxy;
R₄ is hydrogen;
X₁ is CR₆

### X₂ is N;

R₅ is chloro; cyano; difluoromethyl; trifluoromethyl;
R₆ is hydrogen; oxo; methoxy; 1,2,3-triazole-2-yl; N-methylaminocarbonyl, N,N-dimethylaminocarbonyl; pyrrolidin-1-yl carbonyl and
R₈ is hydrogen.

Embodiment 2017-4 employs a MALT1 inhibitor of embodiment 2017-1 or a pharmaceutically acceptable salt thereof, wherein

R₁ is methyl, fluoro or chloro;
R₂ is C₁-C₆ alkyl;
R₃ is C₁-C₆ alkoxy;
R₄ is hydrogen;
X₁ is CR₆

### X₂ is N;

R₅ is chloro; cyano; difluoromethyl; trifluoromethyl;
R₆ is hydrogen; methoxy; 1,2,3-triazole-2-yl; N-methylaminocarbonyl, N,N-dimethylamino carbonyl; pyrrolidin-1-yl carbonyl and
R₈ is hydrogen.

Embodiment 2017-5 employs a MALT1 inhibitor of embodiment 2017-1 or a pharmaceutically acceptable salt thereof, wherein

R₁ is methyl, fluoro or chloro;
R₂ is C₁-C₆ alkyl;
R₃ is C₁-C₆ alkoxy;
R₄ is hydrogen;

### X₁-is N;

X₂ is CR₇;
R₅ is chloro; cyano; difluoromethyl; trifluoromethyl;
R₇ is hydrogen; and
R₈ is hydrogen.

Embodiment 6-2017 employs a MALT1 inhibitor of embodiment 2017-1 or a pharmaceutically acceptable salt thereof, wherein

R₁ is fluoro or chloro;
R₂ is C₁-C₆ alkoxy;
R₃ is C₁-C₆ alkyl;
R₄ is hydrogen;
X₁ is CR₆

### X₂ is N;

R₅ is chloro; cyano; difluoromethyl; trifluoromethyl;
R₆ is hydrogen; methoxy; 1,2,3-triazole-2-yl; N-methylaminocarbonyl, N,N-imethylamino carbonyl; pyrrolidin-1-yl carbonyl; and
R₈ is hydrogen.

Embodiment 2017-7 employs a MALT1 inhibitor of embodiment 2017-1 or a pharmaceutically acceptable salt thereof, wherein

R₁ is fluoro or chloro;
R₂ is C₁-C₆ alkoxy;
R₃ is C₁-C₆ alkyl;
R₄ is hydrogen;

### X₁ is N;

X₂ is CR₇;
R₅ is chloro; cyano; difluoromethyl; trifluoromethyl;
R₇ is hydrogen; and
R₈ is hydrogen.

Embodiment 2017-8 employs a MALT1 inhibitor of embodiment 2017-1 or a pharmaceutically acceptable salt thereof, selected from
(S)-1-(5-cyanopyridin-3-yl)-3-(7-(1-methoxyethyl)-2-methylpyrazolo[1,5-a]pyrimidin-6-yl)urea;
(S)-1-(2-(difluoromethyl)pyridin-4-yl)-3-(2-fluoro-7-(1-methoxyethyl) pyrazolo[1,5-a]pyrimidin-6-yl)urea;
(S)-1-(2-chloro-7-(1-methoxyethyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-isopropylpyrazolo[1,5-a]pyrimidin-6-yl)urea;
(S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea;
(S)-1-(5-cyano-6-methoxypyridin-3-y)-3-(2-fluoro-7-(1-methoxyethyl) pyrazolo[1,5-a]pyrimidin-6-yl)urea;
(S)-1-(6-(2H-1,2,3-triazol-2-yl)-5-(trifluoromethyl)pyridin-3-yl)-3-(2-chloro-7-(1-(2-methoxyethoxy)ethyl)pyrazolo[1,5-a]pyrimidin-6-yl)urea;
(S)-1-(6-(2H-1,2,3-triazol-2-yl)-5-(trifluoromethyl)pyridin-3-yl)-3-(2-chloro-7-(1-methoxy-2-methyl-propyl)pyrazolo[1,5-a]pyrimidin-6-yl)urea;
1-(2-chloro-7-(1-(methoxymethyl)cyclopropyl)pyrazolo[1,5-a]pyrimidin-6-yl)-3-(5-cyanopyridin-3-yl)urea;
1-(5-chloro-6-(2H-1,2,3-triazol-2-yl)pyridin-3-yl)-3-(2-chloro-7-((1R,2S)-1,2-dimethoxypropyl) pyrazolo[1,5-a]pyrimidin-6-yl)urea;
(S)-1-(2-chloro-7-(1-methoxyethyl)pyrazolo[1,5-a]pyrimidin-6-yl)-3-(5-cyano-6-(2H-1,2,3-triazol-2-yl)pyridin-3-yl)urea;
(S)-1-(5-cyanopyridin-3-yl)-3-(2-fluoro-7-(1-methoxyethyl)pyrazolo[1,5-a]pyrimidin-6-yl)urea;
1-(7-((S)-1-(((R)-1-acetylpyrrolidin-3-yl)oxy)ethyl)-2-chloropyrazolo[1,5-a]pyrimidin-6-yl)-3-(5-chloro-6-(2H-1,2,3-triazol-2-yl)pyridin-3-yl)urea;
(S)-1-(5-chloro-6-(2H-1,2,3-triazol-2-yl)pyridin-3-yl)-3-(2-fluoro-7-(1-methoxy-2-methylpropyl)-pyrazolo[1,5-a]pyrimidin-6-yl)urea;
(S)-1-(5-cyano-6-(2H-1,2,3-triazol-2-yl)pyridin-3-yl)-3-(7-(1-methoxy-2-methylpropyl)-2-methylpyrazolo[1,5-a]pyrimidin-6-yl)urea;
(S)-1-(2-chloro-7-(1-methoxyethyl)pyrazolo[1,5-a]pyrimidin-6-yl)-3-(5-cyano-6-methoxypyridin-3-yl)urea;
1-(2-fluoro-7-((S)-1-methoxyethyl)pyrazolo[1,5-a]pyrimidin-6-yl)-3-(2-(1-hydroxyethyl)-6-(trifluoromethyl)pyridin-4-yl)urea;
(S)-1-(5-cyano-6-(2H-1,2,3-triazol-2-yl)pyridin-3-yl)-3-(2-fluoro-7-(1-methoxyethyl)pyrazolo[1,5-a]pyrimidin-6-yl)urea;
1-(2-chloro-7-(1,2-dimethoxyethyl)pyrazolo[1,5-a]pyrimidin-6-yl)-3-(5-cyano-6-(2H-1,2,3-triazol-2-yl)pyridin-3-yl)urea;
1-(2-chloro-7-((S)-1-methoxyethyl)pyrazolo[1,5-a]pyrimidin-6-yl)-3-(2-(2,2,2-trifluoro-1-hydroxy-ethyl)pyridin-4-y) urea;
(S)-1-(5-chloro-2-(2-methoxyethoxy)pyridin-3-y)-3-(2-chloro-7-(1-methoxyethyl)-pyrazolo[1,5-a]-pyrimidin-6-yl)urea;
(S)-1-(5-cyano-6-methoxypyridin-3-yl)-3-(7-(1-methoxy-2-methylpropyl)-2-methylpyrazolo[1,5-a]-pyrimidin-6-yl)urea;
(S)-1-(2-cyanopyridin-4-yl)-3-(2-fluoro-7-(1-methoxyethyl)pyrazolo[1,5-a]pyrimidin-6-yl)urea;
(S)-1-(5-cyano-6-methoxypyridin-3-yl)-3-(7-(1-methoxyethyl)-2-methylpyrazolo[1,5-a]pyrimidin-6-yl)urea;
1-(2-chloro-7-((1R,2S)-1,2-dimethoxypropyl)pyrazolo[1,5-a]pyrimidin-6-yl)-3-(5-cyano-6-(2H-1,2,3-triazol-2-yl)pyridin-3-yl)urea;
1-(7-((S)-1-(((S)-1-acetylpyrrolidin-3-yl)oxy)ethyl)-2-chloropyrazolo[1,5-a]pyrimidin-6-yl)-3-(5-cyano-6-methoxypyridin-3-yl)urea;
(S)-1-(5-cyano-6-(2H-1,2,3-triazol-2-yl)pyridin-3-yl)-3-(7-(1-methoxyethyl)-2-methylpyrazolo[1,5-a]pyrimidin-6-yl)urea;
(S)-6-chloro-4-(3-(2-chloro-7-(1-methoxyethyl)pyrazolo[1,5-a]pyrimidin-6-yl)ureido)-N,N-dimethylpicolinamide;
(S)-1-(5-(difluoro-methyl)pyridin-3-yl)-3-(2-fluoro-7-(1-methoxyethyl)-pyrazolo[1,5-a]pyrimidin-6-yl)urea;
(S)-1-(2-fluoro-7-(1-methoxyethyl)pyrazolo[1,5-a]pyrimidin-6-yl)-3-(5-(trifluoro-methyl)pyridin-3-yl)urea;
(S)-3-chloro-5-(3-(2-chloro-7-(1-methoxyethyl)pyrazolo[1,5-a]pyrimidin-6-yl)ureido)-N,N-dimethylpicolinamide;
(S)-1-(5-chloro-pyridin-3-yl)-3-(2-fluoro-7-(1-methoxyethyl)pyrazolo[1,5-a]pyrimidin-6-yl)urea;
(S)-1-(5-chloro-6-(pyrrolidine-1-carbonyl)pyridin-3-yl)-3-(2-chloro-7-(1-methoxyethyl)pyrazolo-[1,5-a]pyrimidin-6-yl)urea
(S)-3-chloro-5-(3-(2-chloro-7-(1-methoxyethyl)pyrazolo[1,5-a]pyrimidin-6-yl)ureido)-N-methylpicolinamide
(S)-1-(2-chloro-7-(1-methoxyethyl)pyrazolo[1,5-a]pyrimidin-6-yl)-3-(5-chloropyridin-3-yl)urea;
(S)-1-(7-(1-aminoethyl)-2-chloropyrazolo[1,5-a]pyrimidin-6-yl)-3-(5-chloro-6-(2H-1,2,3-triazol-2-yl)pyridin-3-yl)urea;
(S)-1-(5-cyanopyridin-3-yl)-3-(7-(1-hydroxyethyl)-2-methylpyrazolo[1,5-a]pyrimidin-6-yl)urea;
(S)-1-(2-(difluoromethyl)pyridin-4-yl)-3-(2-fluoro-7-(1-hydroxyethyl) pyrazolo[1,5-a]pyrimidin-6-yl)urea;
1-(2-((S)-2-aminopropoxy)-5-chloropyridin-3-yl)-3-(2-chloro-7-((S)-1-methoxyethyl)pyrazolo[1,5-a]pyrimidin-6-yl)urea;
(S)-2-(difluoromethyl)-4-(3-(2-fluoro-7-(1-methoxyethyl)pyrazolo[1,5-a]pyrimidin-6-yl)ureido)pyridine 1-oxide;
1-(2-chloro-7-((1R,2S)-1,2-dimethoxypropyl)pyrazolo[1,5-a]pyrimidin-6-yl)-3-(5-cyano-6-methoxypyridin-3-yl)urea;
1-(2-chloro-7-(1-(methoxymethyl)cyclopropyl)pyrazolo[1,5-a]pyrimidin-6-yl)-3-(2-cyanopyridin-4-yl)urea; and
(S)-3-chloro-5-(3-(2-fluoro-7-(1-methoxyethyl)pyrazolo[1,5-a]pyrimidin-6-yl)ureido)picolinamide.

Embodiment 2017-9 employs a MALT1 inhibitor of embodiment 1-2017 or a pharmaceutically acceptable salt thereof, wherein X₁ is N and X₂ is not N, or X₁ is not N and X₂ is N.

Embodiment 2017-10 employs a MALT1 inhibitor of formula (III) or a pharmaceutically acceptable salt thereof, wherein

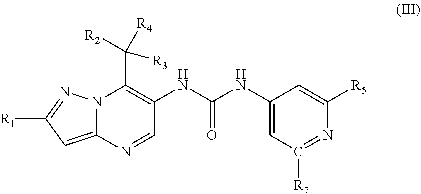

R₁ is fluoro or chloro;
R₂ and R₃ are independently from each other C₁-C₆ alkyl or C₁-C₆ alkoxy;
R₄ is hydrogen;
R₅ and R₇ are independently from each other hydrogen; cyano; halogen or C₁-C₆ alkyl optionally substituted by fluoro and/or hydroxyl.

Embodiment 2017-11 employs a MALT1 inhibitor of formula (IV) or a pharmaceutically acceptable salt thereof, wherein

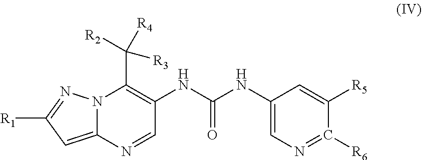

R, is fluoro or chloro;
R₂ and R₃ are independently from each other C₁-C₆ alkyl or C₁-C₆ alkoxy;
R₄ is hydrogen;
R₅ is hydrogen; cyano; halogen or C₁-C₆ alkyl optionally substituted by fluoro and/or hydroxyl; and
R₆ is hydrogen; 1,2,3-triazole-2-yl; N,N-dimethylaminocarbonyl; N-monomethylaminocarbonyl; or pyrrolidin-1-yl carbonyl.

As used herein, the term “C₁-C₆ alkyl” refers to a fully saturated branched or unbranched hydrocarbon moiety having up to 6 carbon atoms. Unless otherwise provided, it refers to hydrocarbon moieties having 1 to 6 carbon atoms, 1 to 4 carbon atoms or 1 to 2 carbon atoms. Representative examples of alkyl include, but are not limited to, methyl, ethyl, n-propyl, isopropyl, n-butyl, sec-butyl, isobutyl, tert-butyl, n-pentyl, isopentyl, neopentyl, n-hexyl and the like.

As used herein, the term “C₁-C₆ alkylene” refers to divalent fully saturated branched or unbranched hydrocarbon moiety having 1 to 6 carbon atoms. The terms “C₁-C₄ alkylene”, “C₁-C₃ alkylene and “C₁-C₂alkylene”, are to be construed accordingly. Representative examples of C₁-C₆ alkylene include, but are not limited to, methylene, ethylene, n-propylene, isopropylene, n-butylene, sec-butylene, iso-butylene, tert-butylene, n-pentylene, isopentylene, neopentylene, and n-hexylene.

As used herein, the term “C₁-C₆ alkoxy” refers to alkyl-O, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like.

Typically, alkoxy groups have about 1 to 6 carbon atoms, 1 to 4 carbon atoms or 1 to 2 carbon atoms.

As used herein, the term “C₁-C₆ alkyl optionally substituted by halogen” refers to C₁-C₆ alkyl as defined above which may be substituted by one or more halogens. Examples include, but are not limited to, trifluoromethyl, difluoromethyl, fluoromethyl, trichloromethyl, 2,2,2-trifluoroethyl, 1-fluoromethyl-2-fluoroethyl, 3-bromo-2-fluoropropyl and 1-bromomethyl-2-bromoethyl.

As used herein, the term “C₁-C₆ alkyl optionally substituted by hydroxyl” refers to C₁-C₆ alkyl as defined above which may be substituted by one or more hydroxy. Examples include, but are not limited to, hydroxymethyl, hydroxyethyl, 1,2-dihydroxyethyl, 2,3-dihydroxypropyl and the like.

As used herein, the term “di C₁-C₆ alkylamino” refers to a moiety of the formula —N(Rₐ)—Rₐ where each Rₐ is a C₁-C₆ alkyl, which may be the same or different, as defined above, in analogy thereto the term “mono C₁-C₆ alkylamino” refers to a moiety of the formula —N(H)— Rₐ where Rₐ is a C₁-C₆ alkyl, which may be the same or different, as defined above.

As used herein, the term “C₃-C₆ cycloalkyl” refers to saturated monocyclic hydrocarbon groups of 3-6 carbon atoms. Cycloalkyl may also be referred to as a carbocyclic ring and vice versa additionally referring to the number of carbon atoms present. Unless otherwise provided, cycloalkyl refers to cyclic hydrocarbon groups having between 3 and 6 ring carbon atoms or between 3 and 4 ring carbon atoms. Exemplary monocyclic hydrocarbon groups include, but are not limited to, cyclopropyl, cyclobutyl, cyclopentyl, and cyclohexyl.

As used herein, the term “employs” means that the respective MALT1 inhibitor is administered to patients suffering from a cancer, in particular a cancer which is essentially unaffected by exposure to pharmaceutically achievable and relevant concentrations of the MALT1 inhibitor, in the furtherance of the immunooncology method or use aspects of the invention.

As used herein, the term “halogen” or “halo” refers to fluoro, chloro, bromo, and iodo; and it may in particular refer to chloro; and it may also in particular refer to fluoro.

As used herein, the term “heterocyclyl” refers to a heterocyclic group that is, unless otherwise indicated, saturated or partially saturated and is preferably a monocyclic or a polycyclic ring (in case of a polycyclic ring particularly a bicyclic, tricyclic or spirocyclic ring); and has 3 to 24, more preferably 4 to 16, most preferably 5 to 10 and most preferably 5 or 6 ring atoms; wherein one or more, preferably one to four, especially one or two ring atoms are a heteroatom (the remaining ring atoms therefore being carbon). The bonding ring (i.e. the ring connecting to the molecule) preferably has 4 to 12, especially 5 to 7 ring atoms. The term heterocyclyl excludes heteroaryl. The heterocyclic group can be attached at a heteroatom or a carbon atom. The heterocyclyl can include fused or bridged rings as well as spirocyclic rings. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorphoiine, and the like.

A substituted heterocyclyl is a heterocyclyl group independently substituted by 1-4, such as one, or two, or three, or four substituents.

As used herein, the term “heteroaryl” refers to a 5-14 membered monocyclic- or bicyclic- or tricyclic-aromatic ring system, having 1 to 8 heteroatoms. Typically, the heteroaryl is a 5-10 membered ring system (e.g., 5-7 membered monocycle or an 8-10 membered bicycle) or a 5-7 membered ring system. Typical heteroaryl groups include 2- or 3-thienyl, 2- or 3-furyl, 2- or 3-pyrrolyl, 2-, 4-, or 5-imidazolyl, 3-, 4-, or 5-pyrazoiyi, 2-, 4-, or 5-thiazolyl, 3-, 4-, or 5-isothiazoiyl, 2-, 4-, or 5-oxazolyl, 3-, 4-, or 5-isoxazoiyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, and 2-, 4-, or 5-pyrimidinyl.

The term “heteroaryl” also refers to a group in which a heteroaromatic ring is fused to one or more aryl, cydoaliphatic, or heterocyclyl rings, where the radical or point of attachment is on the heteroaromatic ring. Nonlimiting examples include 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinolyl, 1-, 3-, 4-, 5-, 6-, 7-, or 8-isoquinolyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4aH carbazolyl, 1-, 2-, 3-, 4-, 5-, 6-, 7-, or 8-carbazolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenanthrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or 1-, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c3carbazolyl,2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-,3-, 5-, or 6-imidazo[2,1-b]thiazolyl, -, 3-, 6-, 7-, 8-, or 9-furo[3,4-c]cinnolinyl, 1-, 2-, 3-,

4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroaryl groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, and 2-, 4-, 5-, 6-, or 7-benzothiazolyl. A substituted heteroaryl is a heteroaryl group containing one or more substituents.

As used herein, the term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl.

A substituted aryl is an aryl group substituted by 1-5 (such as one, or two, or three) substituents independently selected from the group consisting of hydroxyl, thiol, cyano, nitro, C₁-C₄ alkyl, C₁-C₄ alkenyl, C₁-C₄alkynyl, C₁-C₄ alkoxy, C₁-C₄ thioalkyl, C₁-C₄ alkenyloxy, C₁-C₄ alkynyloxy, halogen, C₁-C₄ alkylcarbonyl, carboxy, C₁-C₄ alkoxycarbonyl, amino, C₁-C₄ alkylamino, di-C₁-C₄ alkylamino, C₁-C₄ alkylaminocarbonyl, di-C₁-C₄ alkylaminocarbonyl, C₁-C₄ alkylcarbonylamino, C₁-C₄ alkylcarbonyl (C₁-C₄ alkyl)amino, sulfonyl, sulfamoyl, alkylsulfamoyl, C₁-C₄ alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁-C₄ alkoxy groups.

As used herein, the term a pyridin or a pyridyl optionally substituted by hydroxy e.g. 2-pyridyl, 3-pyridyl, or 4-pyridyl refers to a respective hydroxy-pyridin or hydroxy-pyridyl and may include its tautomeric form such as a respective pyridone or pyridon-yl.

As used herein the term pyridin or pyridyl optionally substituted by oxo e.g. 2-pyridyl, 3-pyridyl, or 4-pyridyl-, refers to a respective pyridone or pyridon-yl and may include its tautomeric form such as a respective hydroxy-pyridin or hydroxy-pyridyl, provided said tautomeric form may be obtainable.

Pyridin or pyridyl optionally substituted by oxo may further refer to a respective pyridine-N-oxide or pyridyl-N-oxide.

As used herein, the terms “salt” or “salts” refers to an acid addition or base addition salt of a compound. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable, in many cases, the compounds are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfafe/sulfafe, camphorsulfonate, chloride/hydrochloride, chlortheophyllonate, citrate, ethandisulfonate, fumarate, gluceptate, gluconate, glucuronate, hippurate, hydroiodide/iodide, isothionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, poiygalacturonate, propionate, stearate, succinate, subsalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ^{1S}N, ¹⁸F ³¹P, ³²P, ³⁵S, ³⁶Cl, ¹²⁵I respectively. Isotopically labeled compounds include for example those into which radioactive isotopes, such as ³H and ¹⁴C, or those into which non-radioactive isotopes, such as ²H and ¹³C are present. Such isotopically labeled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates include those wherein the solvent of crystallization may be isotopically substituted, e.g. D₂O, d₆-acetone, d₆-DMSO.

Compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163,

Any asymmetric atom (e.g., carbon or the like) of the compound(s) of the MALT1 inhibitor can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a MALT1 inhibitor can be in the form of one of the possible isomers, rotamers, atropisomers, tautomers or mixtures thereof, for example, as substantially pure geometric (c/s or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral stationary phase.

Furthermore, the MALT1 inhibitor, including their salts, can also be obtained in the form of their hydrates, or include other solvents used for their crystallization. The MALT1 inhibitor may inherently or by design form solvates with pharmaceutically acceptable solvents (including water); therefore, it is intended that the MALT1 inhibitor embrace both solvated and unsolvated forms. The term “solvate” refers to a molecular complex of a MALT1 inhibitor (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The MALT1 inhibitor, including salts, hydrates and solvates thereof, may inherently or by design form polymorphs.

### Pharmaceutical Compositions and Kits

Any active ingredient described herein, including MALT1 inhibitors, immunomodulatory agents, or compositions comprising tumour antigens (or fragments thereof) may be presented as a pharmaceutical composition said active ingredient and a pharmaceutically acceptable carrier. The pharmaceutical composition can be formulated for particular routes of administration such as oral administration, parenteral administration, and rectal administration, etc. In addition, the pharmaceutical compositions can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, for oral administration the pharmaceutical compositions are tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and/or polyvinylpyrrolidone; if desired d) disintegrants, e.g., starches, agar, alginic acid or its sodium salt, or effervescent mixtures; and/or e) absorbents, colorants, flavors and sweeteners. Tablets may be either film coated or enteric coated according to methods known in the art.

Suitable compositions for oral administration include an effective amount of the active ingredient in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient. Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for intravesical administration include nanocarriers such as solid lipid nanoparticles, protein nanoparticles with targeted ligands grafted on the surface, branched polymeric dendrimers, mucoadhesive biopolymers (such as chitosan), mucoadhesive nanogels or synthetic polymers, magnetic particles, gold nanoshells, and in situ gelling systems. A review is found at Zacche et al, Research and Reports in Urology 2015:7 169-178.

A suitable approach is the use of hydrogels as depot formulations on the bladder walls. An example is thermosensitive hydrogels such as aqueous solutions of poly (ethylene glycol-b-[dl-lactic acid-co-glycolic acid]-b-ethyleneglycol) triblock copolymers that form a free-flowing solution at room temperature and become a viscous gel at body temperature of 37° C. Additionally, liposomal vesicles shown to enhance the therapeutic index of chemotherapeutic agents may be used. A reservoir-based intravesical devices that can be inserted and remain in the bladder may also be used. The drug is then released from the device in a controlled and extended manner. The device can be either biodegradable or nondegradable.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Another embodiment presents anhydrous pharmaceutical compositions and dosage forms comprising the active ingredients, since water may facilitate the degradation of certain compounds. Anhydrous pharmaceutical compositions and dosage forms can be prepared using anhydrous or low moisture containing ingredients and low moisture or low humidity conditions.

An anhydrous pharmaceutical composition may be prepared and stored such that its anhydrous nature is maintained. Accordingly, anhydrous compositions are packaged using materials known to prevent exposure to water such that they can be included in suitable formulary kits.

Examples of suitable packaging include, but are not limited to, hermetically sealed foils, plastics, unit dose containers (e. g., vials), blister packs, and strip packs.

The pharmaceutical compositions and dosage forms may comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

In one embodiment, the invention provides an immuno-oncologic kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

### Synthesis of Compounds of Formula I as Exemplary MALT1 Inhibitors

The synthesis of compounds of formula I is extensively described in WO2015181747. In general such compounds are synthesis as outlined in Scheme 1:

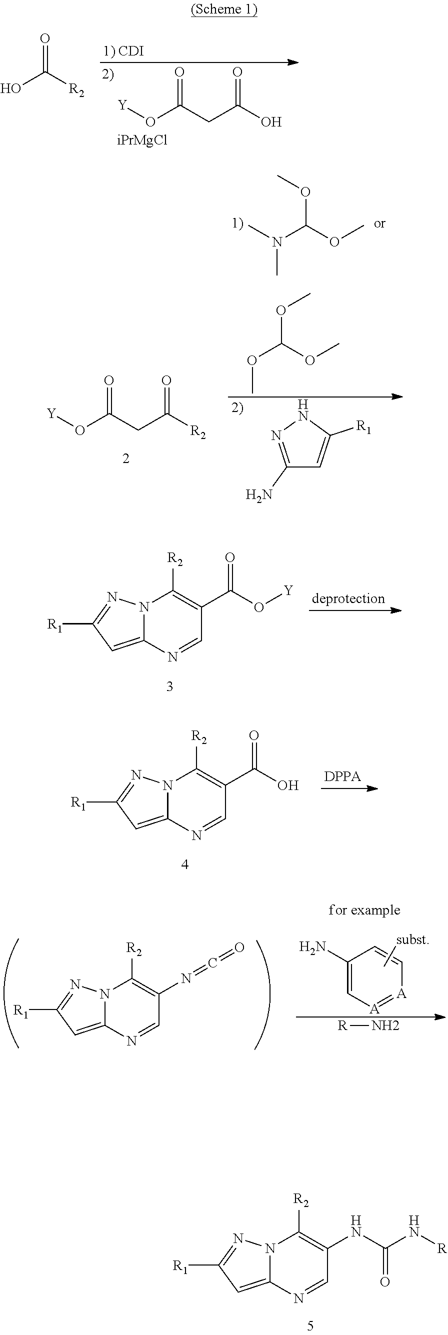

Treatment of an activated acid, e.g. activated as an imidazolid, with the dianion of a malonate mono-ester provides after workup β-ketoester 2. Condensation with a C1 equivalent, e.g. dimethylformamide-dimethylacetal or triethyl orthoformiate, followed by cyclo-condensation with aminopyrazoles in an organic solvent like ethanol at elevated temperature provides the substituted pyrazolo-pyrimidines 3. In case a chiral acid is used in step 1, depending on the substitution pattern, partial racemization may occur during the reaction sequence. In this case the final product may be purified to high enantiomeric purity by chiral chromatography typically as shown in example 119 of WO2015/181747.

Deprotection of the ester provides acid 4. Curtius rearrangement of acid 4 provides an intermediate isocyanate which may be reacted with an appropriate aniline or aminopyridine in a one pot reaction to form the final products.

The synthesis of aminopyrazoles, like 3-amino-5-chioropyrazole can be conducted as follows (Scheme 2):

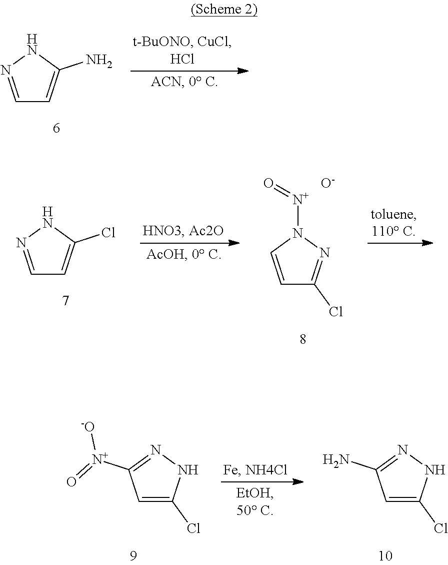

Treatment of aminopyrazole under Sandmeyer conditions provides 3-chloropyrazole. Nitration provides the N-nitropyrazole, which upon heating rearranges to the desired 3-chloro-5-nitropyrazole. Reduction of the nitro group, using iron, tin or tin chloride finally provides the desired 3-amino-S-chloropyrazole 10.

Anilines and aminopyridines used in formula I can be prepared using the following route:

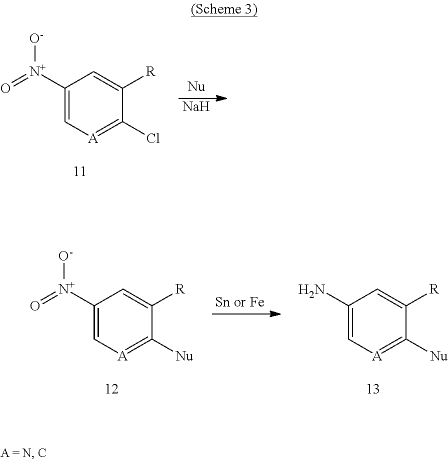

A substituted para-nitro-chlorobenzene or p-nitrochloropyridine is treated with a nucleophile in an inert solvent like DMF, to give the substitution product 12. The nucleophile in this case can be deprotonated alcohols, amines, lactams or heterocycles, e.g. the anion of 1,2,3 triazole. Finally reduction of the nitro substituent using tin or iron in acidic media provides the desired aminophenyl- or aminopyridyl-derivatives 13.

Alternatively, anilines or aminopyridines can be prepared via Curtius rearrangement of suitable aryl acids (Scheme 4):

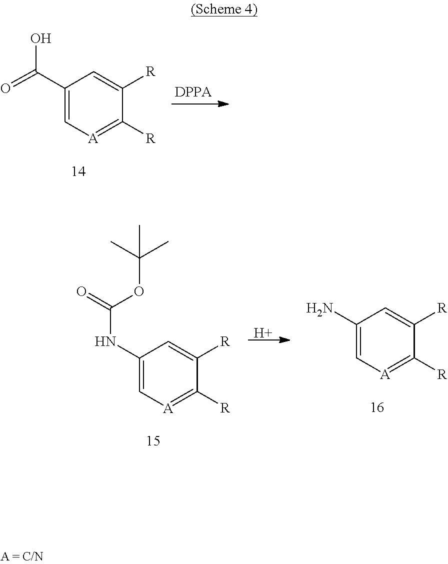

Treatment of acid 4 with diphenyl phosphoryl azide and base in t-butanol provides the i-butoxy-carbonyl-protected amino compound 15, which can be deprotected under acidic conditions using HCl or TFA to give the desired aniline/aminopyridine 16.

Certain aminopyridines and anilines can be prepared by palladium-catalyzed coupling of an aryl halide with a boronic acid according to Scheme 5:

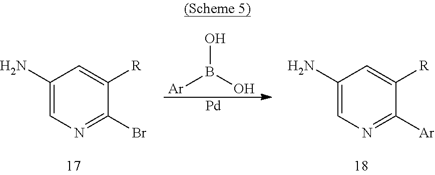

Pyridones of formula I are generally prepared via alkylation of hydroxy pyridines (Scheme 6):

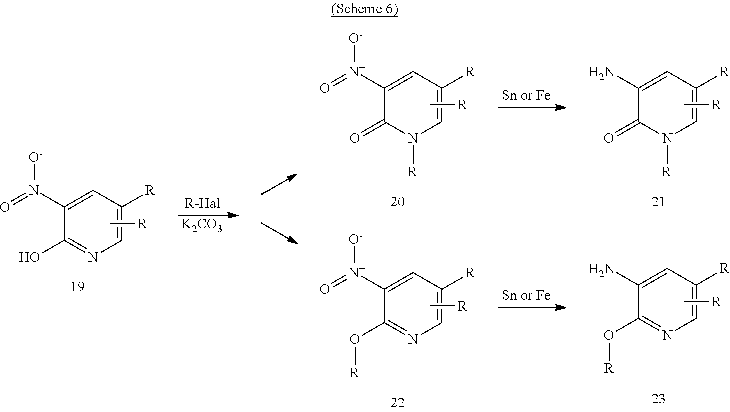

### Broadly Similar Syntheses are Applicable to the MALT1 Inhibitors of Formula II, III and IV, as Described in Detail in WO2017/081641.

The invention is illustrated by the following Examples.

### EXAMPLES

The compounds of the invention including intermediates were prepared as described in the Examples and in the general schemes herein. It will be apparent to a skilled person that analogous synthetic routes may be used, with appropriate modifications, to prepare the compounds of the invention as described herein. The progress of the reactions were followed as appropriate by e.g. LC, GC or TLC, and as the skilled person readily will realise, reaction times and temperatures may be adjusted accordingly. Reactions were performed in an inert atmosphere (including but not limited to nitrogen or argon) where necessary to protect reaction components from air or moisture. The reactants used in the examples were be obtained from commercial sources or prepared from commercially available starting materials as described herein or by methods known in the art.

### Intermediate 1

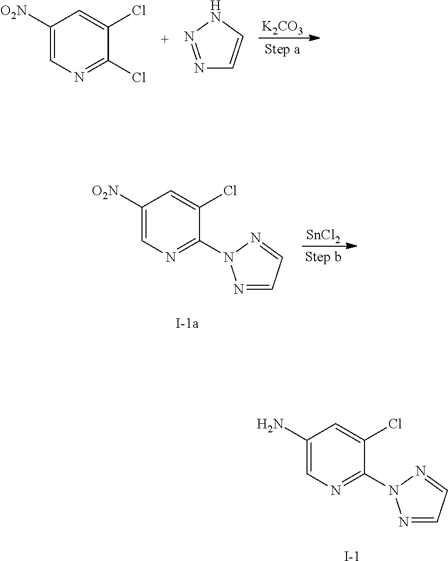

### Step a) 3-Chloro-5-nitro-2-(2H-1,2,3-triazol-2-yl)pyridine (I-1a)

Potassium carbonate (14.0 g, 101.35 mmol) was added to solution of 2,3-dichloro-5-nitropyridine (10.0 g, 51.82 mmol) in THF (60 mL) followed by addition of 2H-1,2,3-triazole (3.4 mL, 58.7 mmol). The resulting mixture was stirred at rt until reaction was deemed completed as judged by TLC (16 h), then diluted with water (300 mL). The aqueous layer was extracted with EtOAc (2×300 mL), the organic layer was dried over sodium sulphate, filtered and concentrated under reduced pressure. The crude product was purified by column chromatography on silica (100-200 mesh), eluted at 20% EtOAc in p. ether which gave the title compound (7.0 g, 60%) as a solid with 99.42% LCMS purity. MS (ES+) 226.03 [M+H]⁺.

### Step b) 5-chloro-6-(2H-1,2,3-triazol-2-yl)pyridin-3-amine (I-1)

Tin(II) chloride (30.0 g, 158 mmol) was added portion wise at rt to a solution of compound I-1a (7.0 g, 31 mmol) in 1M HCl in MeOH (158 mL). The resulting mixture was stirred at rt for 3 h, then concentrated under reduced pressure. The residue was diluted with DCM (100 mL) and the mixture was basified with 1N aqueous NaOH solution (50 mL). The phases were separated and the organic phase was dried over sodium sulphate, filtered and concentrated, which gave the title compound (5.0 g, 77%) as a solid MS (ES+) 196.02 [M+H]⁺.

### Intermediate 2

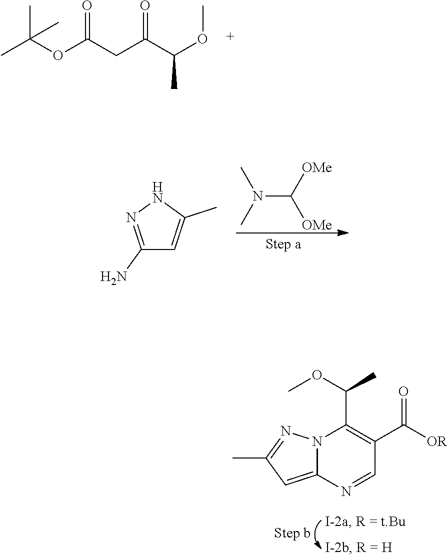

### Step a) (S)-Tert-butyl 7-(1-methoxyethyl)-2-methylpyrazolo[1,5-a]pyrimidine-6-carboxylate (I-2a)

A mixture of (S)-tert-butyl 4-methoxy-3-oxopentanoate (23 g, 0.06 mol) and 1,1-dimethoxy-N,N-dimethylmethanamine (6.2 g, 52 mmol) was heated at 120° C. for 90 min, then a solution of 5-methyl-1H-pyrazol-3-amine (5 g, 51 mmol) in EtOH (20 mL) was added and the reaction mixture was heated at 85° C. for 4 h, then concentrated under reduced pressure.

The obtained crude material was purified by column chromatography on silica eluting with 0-10% EtOAc in p. ether which gave the title compound (12 g, 70%) as a solid. MS (ES+) 292.23 [M+H]⁺.

### Step b) (S)-7-(1-Methoxyethyl)-2-methylpyrazolo[1,5-a]pyrimidine-6-carboxylic acid (I-2b)

TFA (60 mL, 672 mmol) was added under nitrogen at rt to a stirred solution of I-2a (12 g, 35.8 mmol) in DCM (100 mL). The reaction mixture was stirred at rt for 6 h, then concentrated under reduced pressure. Diethyl ether (20 mL) was added to the residue and the mixture was concentrated and dried. The afforded crude compound was triturated with diethyl ether (10 mL) which gave the title compound (6.8 g, 78%) MS (ES+) 234.16 [M−H]⁺.

### Intermediate 3

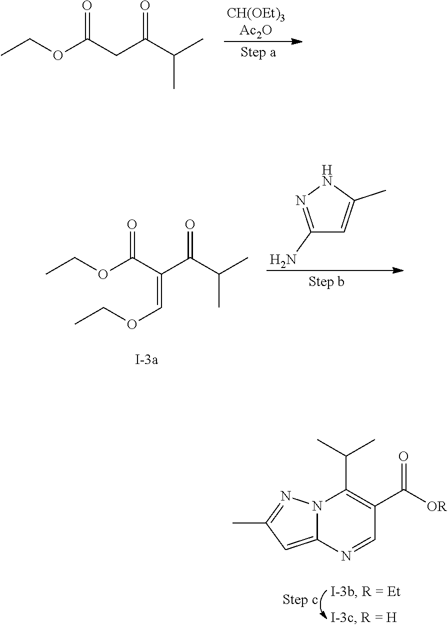

### Step a) Ethyl 7-isopropyl-2-methylpyrazolo[1,5-a]pyrimidine-6-carboxylate (I-3a)

A solution of ethyl 4-methyl-3-oxopentanoate (20 g, 0.13 mol), triethoxymethane (41 g, 0.28 mol) and acetic anhydride (22 mL, 0.28 mol) was stirred at 135° C. for 16 h, then concentrated under reduced pressure, which gave the title compound (20 g, 69.71%) as a liquid. MS (ES+) 215.15 (M+H) at 2.61 RT.

### Step b) Ethyl 7-isopropyl-2-methylpyrazolo[1,5-a]pyrimidine-6-carboxylate (I-3b)

A solution of (Z)-ethyl 2-(ethoxymethylene)-4-methyl-3-oxopentanoate (15 g, 53 mol) and 3-amino-5-methylpyrrazole in EtOH (250 mL) was stirred at 80° C. for 16 h, then concentrated under reduced pressure. The obtained crude was diluted with water (100 mL) and extracted with EtOAc (2×250 mL), the combined organic layers were washed with aqueous saturated sodium bicarbonate (100 mL), water (100 mL), dried (Na₂SO₄), filtered and concentrated which gave the title compound (10 g, 48%) as a solid. MS (ES+) 248.15 [M+H]⁺.

### Step c) 7-isopropyl-2-methylpyrazolo[1,5-a]pyrimidine-6-carboxylic acid (I-3c)

2M NaOH (25 mL) was added at rt to a solution of I-3b (10 g, 25.3 mmol) in EtOH (100 mL). The reaction mixture was stirred at 60° C. for 5 h, then concentrated under reduced pressure. The afforded crude was diluted with water (50 mL) and extracted with EtOAc (3×100 mL). The aqueous layer was acidified with 1 M aqueous HCl and the solid formed was filtered and washed with water (2×25 mL) and dried under vacuum which gave the title compound (5 g, 87%) as a solid. MS (ES+) 220.15 [M+H]⁺.

### Example 1

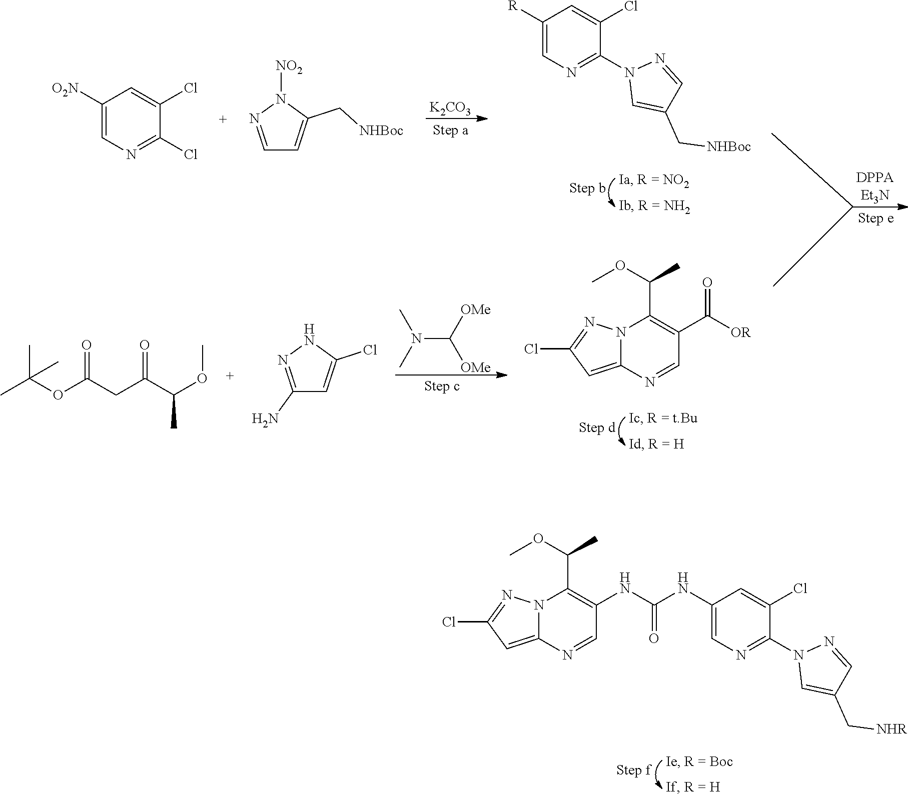

### Step a) tert-butyl ((1-(3-chloro-5-nitropyridin-2-yl)-1H-pyrazol-4-yl)methyl)carbamate (1a)

To solution of 2,3-dichloro-5-nitropyridine (400 mg, 2.07 mmol) in DMF (10 mL) was added potassium carbonate (350 mg, 2.53 mmol) followed by tert-butyl ((1H-pyrazol-4-yl)methyl)carbamate (410 mg, 2.08 mmol), the resulting mixture was stirred at room temperature for 6 h, then diluted with Water (20 mL). The aqueous layer was extracted with EtOAc (2×50 mL). The combined organic layers were dried over sodium sulfate, filtered and concentrated under reduced pressure. The crude product was purified by column chromatography on silica gel eluted with 10-20% EtOAc in p. ether which gave the title compound (520 mg, 66% yield) a solid. MS (ES+) 354.10 [M+H]⁺.

### Step b) tert-butyl ((1-(5-amino-3-chloropyridin-2-yl)-1H-pyrazol-4-yl)methyl)carbamate (1b)

Iron powder (410 mg, 7.34 mmol) was added portion-wise at room temperature to a solution of compound 1b (520 mg, 1.47 mmol) in acetic acid (5 mL). The resulting mixture was heated to 80° C. for 10 min, then the reaction was quenched with NaHCO₃ solution (30 mL) and the mixture was extracted with EtOAc (50 mL). The organic phase was separated and the aqueous phase was extracted with EtOAc (50 mL). The combined organic layers were washed with brine (20 mL), dried over Na₂SO₄ and concentrated which gave the title compound (450 mg, 85%) as a solid. The compound was used in next step without further purification. MS (ES+) 324.32 [M+H]⁺.

### Step c) (S)-tert-butyl 2-chloro-7-(1-methoxyethyl)pyrazolo[1,5-a]pyrimidine-6-carboxylate (1c)

A mixture of (S)-tert-butyl 4-methoxy-3-oxopentanoate (5.5 g, 27.2 mmol) and 1,1-dimethoxy-N,N-dimethylmethanamine (3.2 g, 26.8 mmol) was heated at 120° C. for 90 min, then a solution of 5-chloro-1H-pyrazol-3-amine (3.3 g, 28.1 mmol) in ethanol (60 ml) was added and the reaction mixture was heated to 85° C. for 2 h. The reaction mixture was concentrated under reduced pressure and the obtained crude material was purified by column chromatography on silica gel eluting with 0-10% EtOAc in p. ether which gave the title compound (5.5 g, 64%) as a solid.

### Step d) (S)-2-chloro-7-(1-methoxyethyl)pyrazolo[1,5-a]pyrimidine-6-carboxylic acid (1d)

Trifluoroacetic acid (30 mL) was added under nitrogen at room temperature to a stirred solution of compound 1c (6.0 g, 19.2 mmol) in DCM (60 mL). The reaction mixture was stirred at room temperature for 16 h, then concentrated under reduced pressure. Diethyl ether (20 mL) was added to the crude compound and the mixture concentrated on rotavapour and dried which gave the title compound (3.5 g, 68%). MS (ES+) 312.09 [M+H]⁺.

### Step e) (S)-tert-butyl ((1-(3-chloro-5-(3-(2-chloro-7-(1-methoxyethyl)pyrazolo[1,5-a]pyrimidin-6-yl)ureido)pyridin-2-yl)-1H-pyrazol-4-yl)methyl)carbamate (1e)

Diphenylphosphoryl azide (0.36 mL, 1.670 mmol) was added to a solution of compound 1d (350 mg, 1.37 mmol) in 1,4-dioxane (10 mL) followed by addition of triethylamine (0.98 mL, 7.07 mmol). The mixture was stirred under nitrogen at room temperature for 30 min, then a solution of compound 1b (440 mg, 1.36 mmol) was added and the mixture was heated at 100° C. for 2 h. The reaction mixture was diluted with water (30 mL) and extracted with EtOAc (2×30 ml). The organic layer was dried over sodium sulfate, filtered and concentrated under reduced pressure. The crude residue was combined with another batch and purified by column chromatography on silica gel eluting with a gradient of 2% MeOH in DCM which gave the title compound (310 mg, 35%) as a solid. MS (ES+) 576.39 [M+H]⁺.

### Step f) (S)-1-(6-(4-(aminomethyl)-1H-pyrazol-1-yl)-5-chloropyridin-3-yl)-3-(2-chloro-7-(1-methoxyethyl)pyrazolo[1,5-a]pyrimidin-6-yl)urea (1f)

Trifluoroacetic acid (0.2 mL, 2.613 mmol) was added dropwise at 0° C. under N₂ to a stirred solution of compound 1e (310 mg, 0.491 mmol) in DCM (10 mL). The resulting reaction mixture was stirred at room temperature for 2 h, then concentrated and saturated sodium bicarbonate solution was added. The pH was adjusted to pH 8. The mixture was extracted with DCM (2×30 mL), the combined organic layers were washed with brine (20 mL), dried over anhydrous Na₂SO₄ and concentrated under vacuum. The crude product was purified by Prep C18 HPLC using eluted with 10 mM ammonium bicarbonate in H₂O:acetonitrile, which gave the title compound (55 mg, 13%) as a solid. MS (ES+) 476.10 [M+H]⁺.

### Example 2

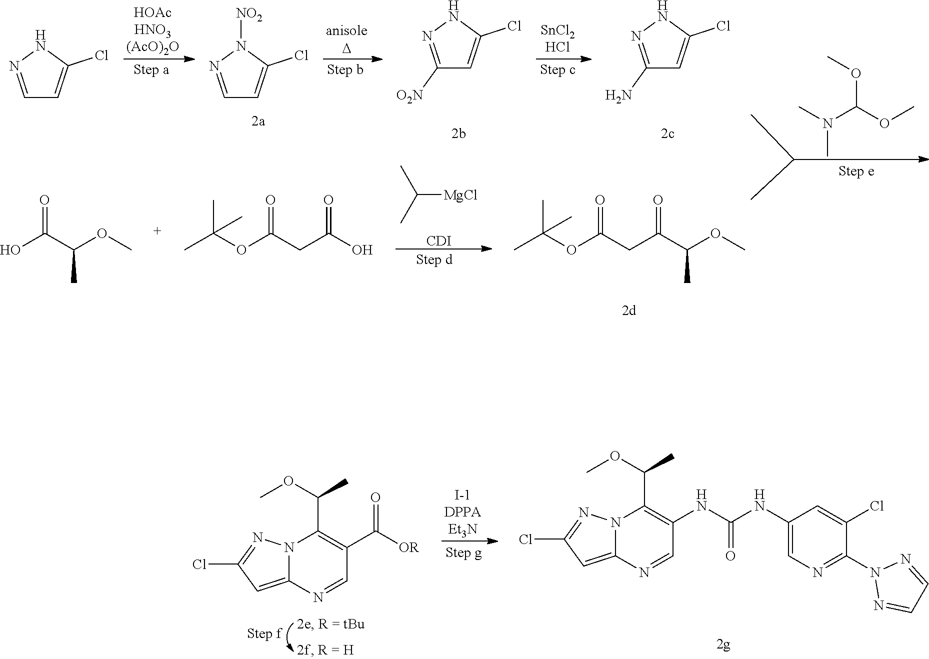

### Step a) 5-chloro-1-nitro-1H-pyrazole (2a)

Fuming nitric acid (14.00 ml, 329 mmol) was added at 0° C. over a period of 10 min to a solution of 5-chloro-1H-pyrazole (10.0 g, 97.5 mmol) in acetic acid (14.0 ml, 245 mmol). The resulting mixture was stirred at 0° C. for 2 h, then acetic anhydride (33.0 ml, 349 mmol) was added and the reaction mixture was stirred at rt. Progress of the reaction was monitored by TLC and LCMS and when starting material was deemed completely consumed (after 4 h), the reaction mixture was poured into ice-water (70 mL) and basified with Na₂CO₃ (60 g) and extracted with EtOAc (3×100 mL). The combined organic layers were washed with aqueous saturated sodium bicarbonate (100 mL) and brine (50 mL), dried over Na₂SO₄ and concentrated. The afforded solid was washed with n-pentane which gave the title compound (7.0 g, 6.2%) as a solid. MS (ES+) 147.93 [M+H]⁺.

### Step b) 5-chloro-3-nitro-1H-pyrazole (2b)

Compound 2a (7.0 g, 47.4 mmol) was dissolved in anisole (150 ml) in a steal bomb. The vessel was sealed and the mixture heated at 140° C. The progress of the reaction was monitored by TLC and stopped after 16 h even though starting material was not completely consumed. The mixture was filtered and the filtrate was concentrated under vacuum. The residue was purified by column chromatography on silica gel (100-200 mesh) eluted with 20% EtOAc in p. ether which gave the title compound (4.0 g, 56%) as a solid. MS (ES−) 145.99 [M−H]⁻.

### Step c) 5-chloro-1H-pyrazol-3-amine (2c)

Aqueous HCl (16.8 mL, 544 mmol) was added over a period of 10 min to a solution of compound 2b (4.0 g, 27.1 mmol) in MeOH (200 mL). The reaction mixture was cooled to 0° C. and tin(II) chloride (30.0 g, 158 mmol) was added portion wise and the resulting reaction mixture was stirred at rt. The progress of the reaction was monitored by TLC and after 16 h, when starting material was deemed consumed, the solvent was evaporated. The residue was diluted with EtOAc (100 mL) and 30% aqueous NaOH solution (120 mL) was added dropwise at 0° C. until basic pH, then stirred at 0° C. for 2 h. Solid precipitates were filtered off through a pad of Celite and the cake was rinsed with EtOAc (50 mL) and water (50 mL). The organic phase was separated and the aqueous phase was extracted with EtOAc (50 mL). The combined organic layers were washed with brine (50 mL), dried over Na₂SO₄, filtered and concentrated under vacuum which gave the title compound (3.5 g, 93%). The compound was used in next step without further purification. MS (ES+) 118.04 [M+H]⁺.

### Step d) (S)-tert-butyl 4-methoxy-3-oxopentanoate (2d)

N,N′-carbonyldiimidazole (17.0 g, 105 mmol) was added 0° C. to a solution of (S)-2-methoxypropanoic acid (10.0 g, 96.1 mmol) in THF (200 mL) and the mixture was stirred at rt for 3 h. In a separate flask, 2M isopropyl magnesium chloride in THF (140 mL, 280 mmol) was added dropwise at 0° C. to a solution of 3-(tert-butoxy)-3-oxopropanoic acid (23.0 g, 143 mmol) in THF (200 mL) and the reaction mixture was stirred for 3 h rt. This second solution was added drop wise to the first solution at 0° C. and the resulting mixture was stirred at rt for 1 h. The reaction was monitored by TLC and when deemed completed it was quenched with 10% aqueous citric acid solution (100 mL). The mixture was diluted with water (250 mL) and the aqueous layer was extracted with EtOAc (2×500 ml). The combined organic layers were washed with saturated sodium bicarbonate solution (200 mL), dried over sodium sulphate filtered and concentrated under reduced pressure. The obtained crude material was purified by column (100-200 silica gel) eluted with 0-10% EtOAc in p. ether. Fractions containing the pure compound was concentrated which gave the title compound as a liquid (13.2 g, 60%). MS (ES+) 201.11 [M+H]⁺.

### Step e) (S)-tert-butyl 2-chloro-7-(1-methoxyethyl)pyrazolo[1,5-a]pyrimidine-6-carboxylate (2e)

A mixture of compound 2d (5.5 g, 27 mmol) and 1,1-dimethoxy-N,N-dimethylmethanamine (3.2 g, 27 mmol) was heated at 120° C. for 90 min, then a solution of compound 1c (3.3 g, 28 mmol) in EtOH (60 ml) was added and the reaction mixture was heated to 85° C. for 2 h. When TLC indicated absence of compound 1d and formation of product, the mixture mass was concentrated under reduced pressure. The obtained crude material was purified by column chromatography (100-200 mesh silica) eluted with 0-10% EtOAc in p. ether. Fractions containing the pure compound were combined, concentrated and dried in vacuo which gave the title compound (5.5 g, 64%) as a solid. MS (ES+) 312.08 [M+H]⁺.

### Step f) (S)-2-chloro-7-(1-methoxyethyl)pyrazolo[1,5-a]pyrimidine-6-carboxylic acid (2f)

Trifluoroacetic acid (30 mL) was added at rt under nitrogen to a stirred solution of compound 1e (6.0 g, 19 mmol) in DCM (60 mL). The reaction mixture was stirred at rt for 16 h, then concentrated under reduced pressure. Diethyl ether (20 mL) was added to the afforded crude compound and the mixture was concentrated and dried, which gave the title compound as a (3.5 g, 68%). MS (ES+) 256.09 [M+H]⁺.

### Step g) (S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea (2q)

DPPA (3.0 mL, 14 mmol) and Et₃N (8.0 mL, 5 mmol) were added to a solution of compound 2f (3.0 g, 11 mmol) in 1,4-dioxane (20 mL). The solution was stirred at rt for 30 min, then compound 1-1 (3.5 g, 17 mmol) was added and the reaction mixture was stirred for 2 h at 100° C. When TLC indicated complete consumption of compound if, the reaction mixture was cooled to rt, water (50 mL) was added and the mixture was extracted with EtOAc (2×100 mL). The combined organic layer was washed with aqueous saturated sodium bicarbonate (50 mL), dried over Na₂SO₄, filtered and concentrated. The afforded crude material was purified by column chromatography on silica gel 100-200 mesh eluted with 3-5% MeOH in DCM. Appropriate fractions were pooled and concentrated and the afforded solid was refluxed in MeOH (20 mL), cooled to rt to precipitate. The solid was again refluxed in MeOH (20 mL) and cooled to rt to precipitate. The solid was washed with diethyl ether, then purified by prep C18 HPLC eluted with 10 μM Buffers: 10 mM ammonium bicarbonate in H₂O: acetonitrile, which gave the title compound (1.08 g) as a solid. MS (ES+) 448.27 [M+H]⁺.

¹H NMR (500 MHz, DMSO) δ 1.59 (d, 3H), 3.34 (s, 3H), 5.43 (q, 1H), 6.95 (s, 1H), 8.16 (s, 2H), 8.49 (d, 1H), 8.55 (d, 1H), 8.59 (s, 1H), 8.96 (s, 1H), 10.30 (s, 1H).

¹³C NMR (126 MHz, DMSO) δ 17.49, 57.30, 72.96, 95.52, 120.23, 126.18, 127.42, 136.14, 136.87, 138.80, 138.83, 141.56, 144.43, 146.35, 150.02, 153.02.

### Example 3

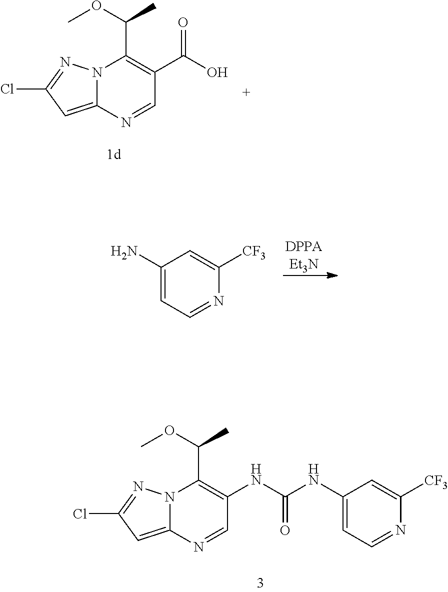 [SMILES: CO[C@@H](C)c1c(C(=O)O)cnc2cc(Cl)nn12 ; Nc1ccnc(C(F)(F)F)c1 ; CO[C@@H](C)c1c(NC(=O)Nc2ccnc(C(F)(F)F)c2)cnc2cc(Cl)nn12]

### (S)-1-(2-Chloro-7-(1-methoxyethyl)pyrazolo[1,5-a]pyrimidin-6-yl)-3-(2-(trifluoromethyl)pyridin-4-yl)urea (3)

Compound 1d (600 mg, 2.0 mmol) was reacted with 2-(trifluoromethyl)pyridin-4-amine (0.761 g, 5.0 mmol) according to the method described in Example 2, step g, which gave the title compound (58 mg, 5.9%). MS (ES+) 415.16 [M+H]⁺.

### Example 4

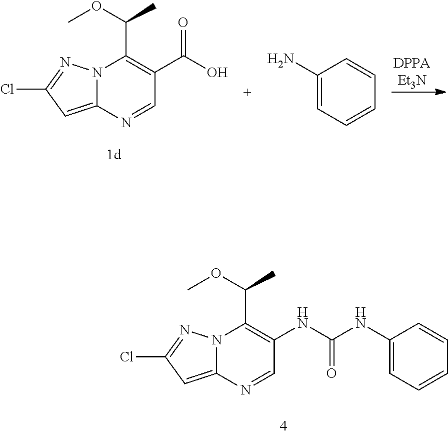 [SMILES: CO[C@@H](C)c1c(C(=O)O)cnc2cc(Cl)nn12 ; Nc1ccccc1 ; CO[C@@H](C)c1c(NC(=O)Nc2ccccc2)cnc2cc(Cl)nn12]

### (S)-1-(2-Chloro-7-(1-methoxyethyl)pyrazolo[1,5-a]pyrimidin-6-yl)-3-phenylurea (4)

Compound 1d (600 mg, 2.0 mmol) was reacted with aniline (99.0 mg, 1.06 mmol) according to the method described in Example 2, step g, which gave the title compound (85 mg, 46%). MS (ES+) 346.18 [M+H]⁺.

### Example 5

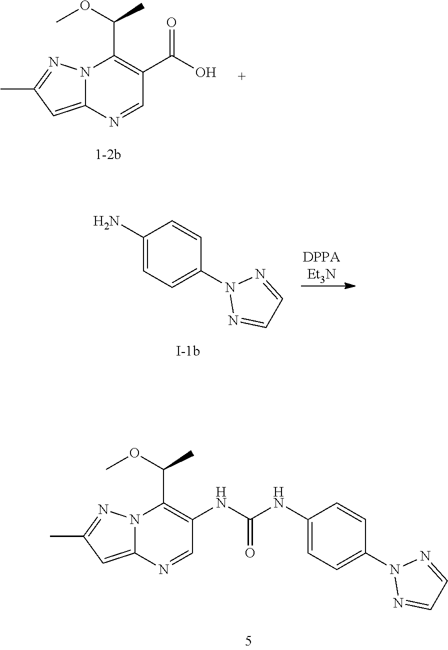 [SMILES: CO[C@@H](C)c1c(C(=O)O)cnc2cc(C)nn12 ; Nc1ccc(-n2nccn2)cc1 ; CO[C@@H](C)c1c(NC(=O)Nc2ccc(-n3nccn3)cc2)cnc2cc(C)nn12]

### (S)-1-(4-(2H-1,2,3-Triazol-2-yl)phenyl)-3-(7-(1-methoxyethyl)-2-methylpyrazolo[1,5-a]pyrimidin-6-yl)urea (5)

Compound I-2b (150 mg, 0.612 mmol) was reacted with I-1b (126 mg, 0.618 mmol) according to the method described in Example 2, step g, which gave the title compound (27 mg, 10%). MS (ES+) 428.22 [M+H]⁺.

### Example 6

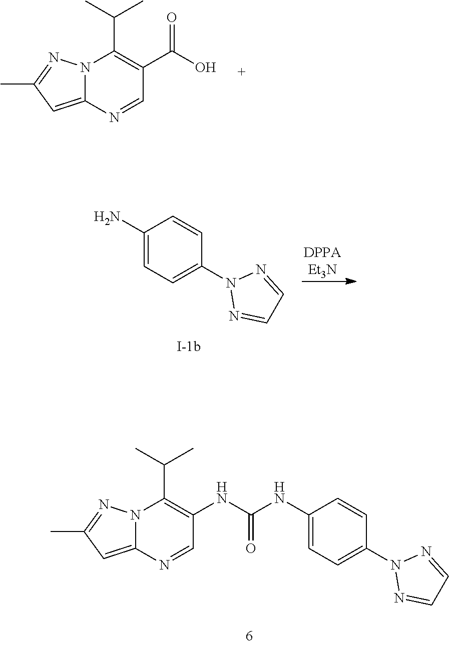 [SMILES: Cc1cc2ncc(C(=O)O)c(C(C)C)n2n1 ; Nc1ccc(-n2nccn2)cc1 ; Cc1cc2ncc(NC(=O)Nc3ccc(-n4nccn4)cc3)c(C(C)C)n2n1]

### 1-(4-(2H-1,2,3-Triazol-2-yl)phenyl)-3-(7-isopropyl-2-methylpyrazolo[1,5-a]pyrimidin-6-yl)urea (6)

7-isopropyl-2-methylpyrazolo[1,5-a]pyrimidine-6-carboxylic acid (100 mg, 0.456 mmol) was reacted with I-1b (90 mg, 0.460 mmol) according to the method described in Example 2, step g, which gave the title compound (50 mg, 23%). MS (ES+) 412.24 [M+H]⁺.

### General Biology Methods

### MALT1 Biochemical Assay

Full-length MALT1 enzyme at 2 nM was assayed in 50 mM HEPES, 100 mM NaCl, 0.9 M NaCitrate, 1 mM EDTA, pH 7.5, with 50 μM Ac-Leu-Arg-Ser-Arg-AMC as substrate. Compound was added in an 11-point concentration series from 100 μM to 1 nM in half-log dilution.

Compounds were distributed in 384 well plates by Echo550, and the assay was run with fluorescence readings at 460 nm, every 2 minutes for 60 minutes.

### MALT1 Cell-Based Activity Assay

Jurkat clone E6-1 cells (ATCC) were grown in standard cell culture conditions (RPMI 1640 with 10% fetal bovine serum, Penicillin 100 U/mL, Streptomycin 0.1 mg/mL). In-cell MALT1 protease activity, was measured as inhibition of PMA/lonomycin-induced cleavage of HOIL1. Day 1 0.75×10⁶ cells/well were seeded in a 48 well plate. Day 2 cells were stimulated by PMA/lonomycin (1:500 dilution) with addition of vehicle or compound in a 5-point concentration series, 1:3 dilution. After 60 minutes cells were washed in PBS and then lysed on ice in CelLytic MT cell lysis reagent (Sigma) with protease and phosphatase inhibitors (2% Protease Inhibitor Cocktail, 10 μM MG-132, 1% Phosphatase Inhibitor Cocktail 1, 1% Phosphatase Inhibitor Cocktail 2, 1% Phosphatase Inhibitor Cocktail 3 (all from Sigma). After a quick centrifugation, supernatant was recovered. Samples were analysed by Protein Simple using the Peggy Sue Master kit for size separation with anti-HOIL1 antibody at 1:200 dilution (Santa Cruz). The approx. 50 kDa cleavage fragment of HOIL1 was used for quantification of protease activity.

Down-stream MALT1 activity was measured by PMA/lonomycin-induced expression of IL-2 in Jurkat (E6-1) cells. Compounds were distributed in a 96 well plate, 8-point concentration series, 1:3 dilution. Jurkat clone E6-1, 4×10⁴ cells/well, were added, and stimulated by PMA/lonomycin (1:500 dilution) for 24 hours at 37° C. Measurement was performed using a MesoScale Sector S600 with the Human IL-2 Tissue Culture kit according to the manufacturers recommendation.

### Quantification of T Cell Populations

Evaluation of T_{reg} and T_{eff} cell numbers was performed by flow cytometry. From a tumour sample (resected piece or biopsy), tumour cells and TILs are dispersed into single cell suspension. Cells are then washed with PBS and stained by antibodies. Samples were stained with antibodies to identify T-cell subpopulations; CD3-BV421 (Biolegend 100228), CD8-FITC (Biolegend 100706), CD4-PE (Biolegend 116005), CD25-PerCP Cy5.5 (Biolegend 101912, clone 3C7), for 15 minutes at room temperature, followed by addition of erythrocyte lysis buffer (cat no. 349202, BD BioSciences, Franklin Lakes, N.J., USA) for 8 minutes. Cells were permeabilized with FoxP3 fix/Perm (cat no. 421401, Biolegend San Diego, Calif., USA), washed with FoxP3 Perm buffer (Cat no. 421402, Biolegend) and stained with an Alexa Fluor® 647 labelled FoxP3 antibody (Cat no. 320013, Biolegend) according to the manufacturer's instructions. The cells were washed twice in PBS with 1% BSA and analyzed on a FACSCanto II (BD Biosciences). Data analysis was performed using FlowJo software (TreeStar, Ashland, Oreg., USA). For intracellular staining of cytokines, inclusion of transport inhibitors e.g. Monensin or Brefeldin A.

Alternatively, immunohistochemical (IHC) evaluation of CD3, CD8, CD4, CD25 and FOXP3 expressing cells is done in Formalin-fixed, paraffin-embedded (FFPE) blocks of biopsies or tumour samples from cancer patients. IHC staining of CD3, CD8, CD4 and FOXP3 is performed with a horseradish-peroxidase technique using a DAKO Autostainer. Antigen retrieval is carried out by the pretreatment of microscope slides with an Epitope Retrieval Solution (Trilog, Cell Marque, Rocklin, Calif., USA) for 30 min. Following that, staining is conducted with standardized Dako EnVision FLEX Peroxidase Blocking reagent (K800, DAKO) and polyclonal antibodies for CD3, CD8, CD4 (dilution 1:50; Dako) and FOXP3 (dilution 1:100: Sigma) following incubation for 120 min at room temperature. Dextran polymer-conjugated horseradish-peroxidase and 3,3′-diaminobenzidine (DAB) chromogen is used for visualisation followed by counterstaining with hematoxylin solution (Gill 3, Sigma). Negative control slides in the absence of primary antibodies are included for each staining.

TIL scoring of tumours by IHC is performed semi-quantitatively by measuring the densities of CD3+, CD8+, CD4+ and FOXP3+ cells, with scores from (1) no, or sporadic cells; (2) moderate numbers of cells; (3) abundant occurrence of cells; to (4) highly abundant occurrence of cells. TILs are evaluated in the following three different areas of the tumour: the intra-epithelial compartment (cells within tumour cell nests); the stroma (cells within the intratumoural stroma) and the tumour periphery (cells localized in tumour periphery). Three random fields are examined, whereas necrotic areas are excluded from the measurements. The total score for CD3, CD8, CD4 and FOXP3 is calculated as the sum of the individual scores from the three tumour areas (intra-epithelial compartment, stroma and tumour periphery), respectively. The total score ranges from 3 to 12, and the median value was used as a cutoff point. The ratios of CD3 and CD8 to both CD4 and FOXP3 (CD3:CD4; CD8:CD4; CD3:FOXP3; and CD8:FOXP3 ratio, respectively) are calculated for each individual tumour based on the cutoff value of each TIL marker.

### Biology Example 1

### Biochemical and Cell Culture Assay of Representative MALT1 Inhibitors.

| | MALT1 | | IL-2 |
|---|---|---|---|
| | biochemical | HOIL1 cleavage | expression assay |
| Compound | assay (IC₅₀ nM) | assay (IC₅₀ μM) | (IC₅₀ μM) |
| Example 1 | 37.5 | 0.04 | 0.03 |
| Example 2 | 9.9 | 0.01 | 0.01 |
| Example 3 | 15.7 | 0.04 | 0.02 |

### Biology Example 2

### Development and Function of Human T-Regulatory Cells

Naïve CD4 T cell sorting and stimulation for regulatory T cell development A flat-bottom 48 well plate was coated with 250 μl of purified functional grade anti-human CD3 at 2 μg/ml in PBS. The plate was sealed and incubated at 37° C. for one hour. Peripheral blood mononuclear cells (PBMC) were isolated from human leucocyte reduction system cones by differential density centrifugation using Ficoll-PLUS (GE Healthcare). Cells were washed three times in RPMI+1% HI-FCS+1% penicillin/streptomycin (P/S) (1% RPMI), then resuspended at a concentration of 200×10⁶/ml of 1% RPMI. An aliquot of cells was set aside for single colour controls, and the sample was stained at a dilution of 1:20 with anti-human CD4, anti-human CD45RA, anti-human CD25, and anti-human CD127 (antibodies from BioLegend and eBioscience) for 20 minutes on ice. Following the incubation, the stained PBMC were washed and resuspended at 50×10₆/ml in PBS+1% HI-FCS. Cells were sorted on a BD FACSARIA high speed cell sorter for the phenotype CD4+CD45RA+CD127+CD25-. Following the sort, cells were resuspended at 4×10₆/ml in pre-warmed cRPMI containing 4×400 U/ml human IL-2 (final concentration 100 U/ml). In each well of the washed anti-CD3-coated 48 well plate, 250 μl of 4×8.0 μg/ml anti-CD28 (eBioscience) and 250 μl of appropriate 4× concentration of compound were added. Either 250 μl of cRPMI or 250 μl of 4×4.0 ng/ml TGFβ were added per well, as appropriate. Finally, 250 μl of cells were added per well. The plate was incubated for 5 days at 37° C. with 5% CO2. After five days, cells were transferred to polypropylene 5 ml tubes and washed twice with cRPMI. Cells were then resuspended in IL-2±TGFβ with the appropriate vehicle control or compound. The plate was incubated for an additional 5 days at 37° C. with 5% CO₂.

Staining Regulatory T Cell Cultures Induced regulatory T cell cultures were washed and counted. Aliquots were stained with Fixable Viability Dye eFluor® 780 (eBioscience) for 30 minutes at 4° C. Then cells were washed with PBS and stained for surface expression of CD25 for 15 minutes at 4° C. After the staining, cells were fixed, permeabilised and stained for intracellular expression of Foxp3 per manufacturer's instructions (Human Foxp3 Buffer set and anti-human Foxp3 antibody from BD Pharmingen). Following staining, cells were washed and resuspended in PBS+1% HI-FCS (FACS buffer). Samples were run within four hours on a CyAn™ ADP Analyzer.

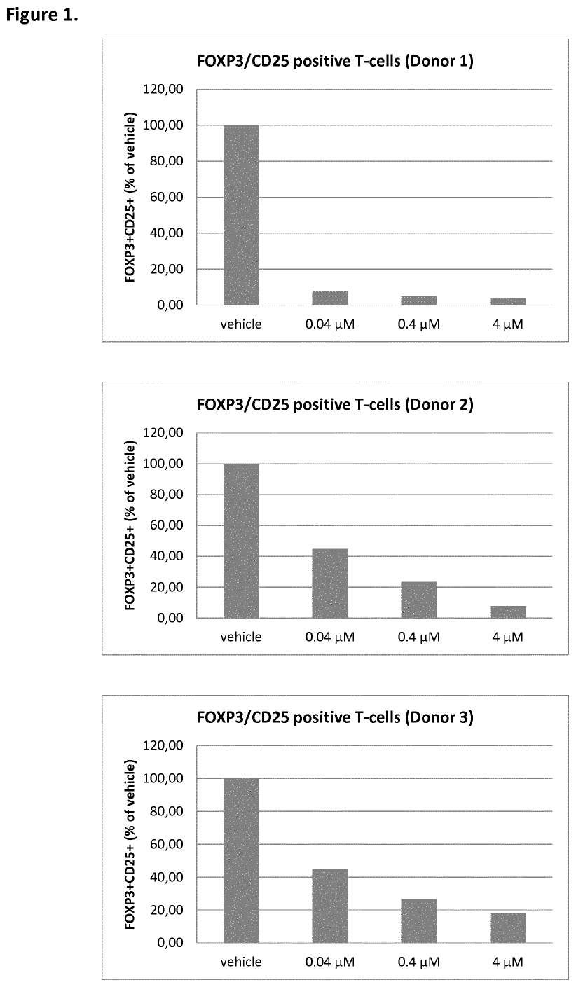

CD4+ naïve T cells cultured for 10 days were analysed for their expression of FoxP3 and CD25. In the IL-2+TGFβ condition, promoting in vitro differentiation of T_{reg} cells, there was a clear induction of a population of viable FoxP3+CD25+ T cells in all three donors (FIG. 1; vehicle).

The addition of Example 2 (0.4 μM, 0.4 μM and 4.0 μM) resulted in a robust inhibition of CD4+FoxP3+CD25+ cell induction, with the highest concentration of drug being most effective (FIG. 1). This impact was present in all 3 donors tested. The conclusion is that MALT1 activity is required for in vitro development of T_{reg} cells, and that this process can be inhibited by applying an appropriate MALT1 inhibitor. The observation also suggests that a MALT1 inhibitor may impact Treg differentiation in tumour tissue or tumour-draining lymph nodes, and thereby reduce the suppression of an anti-tumour immune-response.

### T_{reg} Suppressive Activity

PBMC were isolated from fresh blood by differential density centrifugation using Ficoll-PLUS (GE Healthcare). Cells were washed three times in RPMI+1% heat-inactivated foetal calf serum (HI-FCS)+1% penicillin/streptomycin (P/S) (1% RPMI), then resuspended in PBS+2% heat inactivated foetal calf serum (HI-FCS)+2.0 mM EDTA (isolation buffer). CD4+CD25- and CD4+CD25+ T cells were isolated, per the manufacturer's instructions (Miltenyi CD4+CD25+ Regulatory T cell Isolation Kit, human). Isolated CD4+CD25− T cells were then labeled with Cell Proliferation Dye eFluor® 450 per manufacturer's instructions (eBioscience). An aliquot of cells was stained with anti-CD3 and anti-CD4 (BioLegend and eBioscience) to assess cell purity and dye labeling by flow cytometry. Cultures were plated in cRPMI, with 2.5×104 CD4+CD25− cells per well. Cells from the regulatory T cell cultures were added to achieve ratios of 1 effector: 4 regulatory cells (donors 2-3), 1:2, 1:1, 2:1, 4:1 and 8:1. Anti-CD3/CD28 beads (Dynabeads® T Activator CD3/CD28 beads) were added at a 1:1 ratio with the total number of cells per well. Unstimulated CD4+CD25− T cells and stimulated without regulatory T cells were plated as controls. Cells were incubated for four days at 37° C. with 5% CO₂. After four days, cells were stained with Fixable Viability Dye eFluor® 780 (eBioscience) for 30 minutes at 4° C. Following staining, cells were washed and resuspended in PBS+1% HI-FCS (FACS buffer). Samples were run within four hours on a CyAn™ ADP Analyzer. Immediately prior to acquisition, cells were transferred to FACS tubes with an exact volume of CountBright™ Absolute Counting Beads (ThermoFisher Scientific).

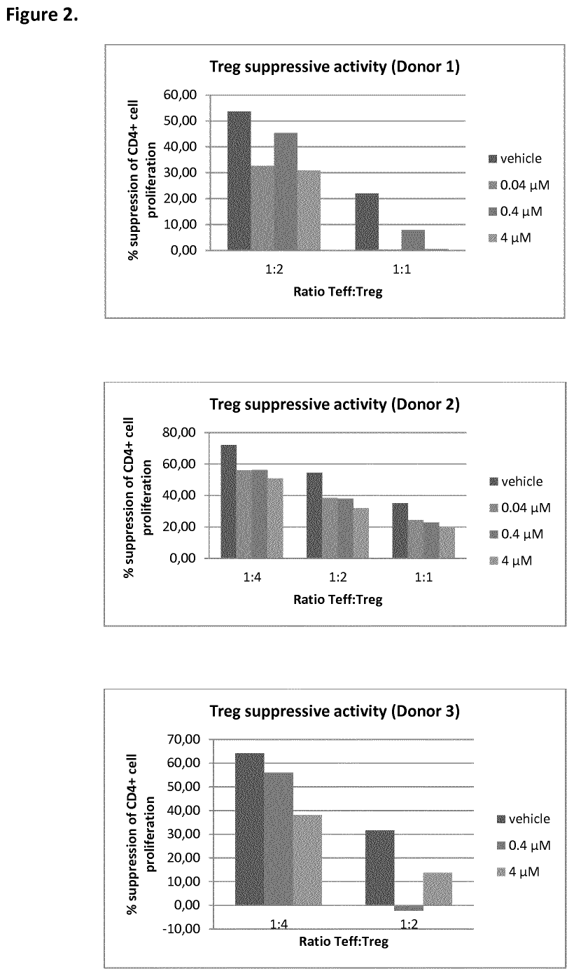

FIG. 2. The proliferation of CD4+CD25− T cells, purified from a different donor and labelled with Cell Proliferation Dye eFluor® 450, was assessed following a 4 day activation with anti-CD3/CD28 beads. There was a robust proliferation of the cells, and they were readily distinguished from unlabeled cultured cells (generated in the 10 day culture period).

The number of proliferating CD4+CD25− “effector” T cells was determined at each ratio (1:4, 1:2, 1:1) of these cells to the cultured cells generated in the previous 10 day culture period (cultured “T_{reg}”). For donor 1, the cultured IL-2+TGFβ differentiated “T_{reg}” cells suppressed effector cell proliferation at a level of 50%. This suppression was reduced in the presence of Example 2 (0.4 μM, 0.4 μM and 4.0 μM) with suppression no longer apparent at a 1:1 ratio. An additional 1:4 ratio (i.e. more T_{reg} cells) was used for donors 2 and 3. The 1:4 ratios were included when determining the suppressive activity of the Treg for these donors as there was an overall lower induction of FoxP3 and CD25 in these cultures as compared to donor 1. For donors 2 and 3, the in vitro generated T_{reg} robustly suppressed the proliferation of the responding effector T cells, and was reduced by increasing concentrations of Example 2.

The reduction in T_{reg} phenotype (FIG. 1) was mirrored in the reduced ability of these cells to suppress the proliferation of the CD4+CD25− effector cells. Although the T_{reg} phenotype was almost completely inhibited there was still some suppressive activity. This is most likely due to the retention of some suppressive activity in the cultured non-T_{reg} cells, despite the culture being taken to day 10 to minimize this impact. Taken together, the results from these 3 donors therefore indicate that MALT1 inhibitor (compound 2g of Example 2 at 0.4 μM, 0.4 μM and 4.0 μM) inhibit the generation of a T_{reg} phenotype and function, with the highest degree of inhibition observed with 4.0 μM.

### Biology Example 3

### Impact on T-Effector Cells in Human Whole Blood

Freshly drawn whole blood from healthy volunteers (2 mL) in a tube was incubated under rotation at 37° C. for 6 hours. Directly after blood sampling, vehicle, 4 μM compound 2g of Example 2, 1 μg/ml of CMV-lysate or combinations thereof were added. After 2 hours Brefeldin-A was added to allow intra-cellular analysis of cytokines (IFNγ and TNFα). Antigen-specific CD8+ T-cells were identified by fluorescence-conjugated tetrameric complexes of HLA-A2 molecules loaded with the NLV peptide epitope (NLVPMVATV) of CMV pp65,

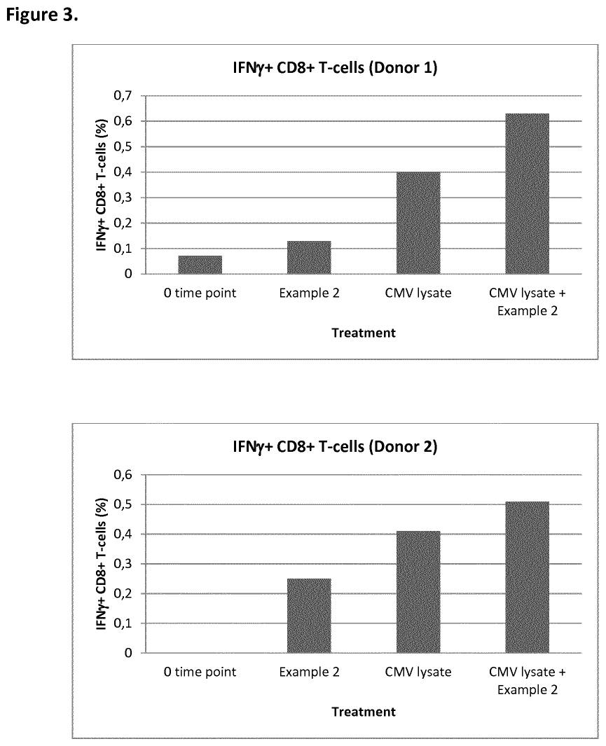

FIG. 3 shows the effect of MALT1 inhibition on CMV-specific (NLV+) CD8+T_{eff} cells expressing IFNγ after stimulation of whole blood with CMV-lysate. In line with the inhibition of T_{reg} differentiation and function by MALT1 inhibitor (FIGS. 1 and 2) the whole blood loop assay demonstrated that MALT1 inhibition caused an increase in activated antigen-specific IFNγ+CD8+ T cells alone, and in combination with CMV lysate. This shows that in a complex immune-response, a MALT1 inhibitor could shift the balance towards a T_{eff} dominated response.

### Biology Example 4

### In Vivo Effects on MB49 Mouse Bladder Cancer

The murine bladder urothelial carcinoma cell line Mouse Bladder (MB)-49 is a C57BL/6-derived cell line cultured at 37 C and 5% CO2 in DMEM+GlutaMax supplemented with 10% FBS, 0.1 mM sodium pyruvate, 100 U/ml Penicillin-Streptomycin. For determining the effects of MALT1 inhibitor on the presence of T-effector and T-regulatory cells in vivo, 3×10⁵ MB49 cells were injected s.c. on the right flank of female C57BL/6 mice. MALT1 inhibitor therapy was administered p.o. once daily on day 8, 9, 10, 11, example 2 at two doses 3 μmol/kg (1.34 mg/kg) or 3 μmol/kg (13.4 mg/kg). As reference 200 μg of anti-CD25 antibody (clone PC61) in 100 μL PBS was administered i.v. on day 8 and 11. The levels of T_{reg} and T_{eff} cells were analyzed by flow cytometry in the tumour (T) and in tumour-draining lymph nodes (TDLN) on day 12.

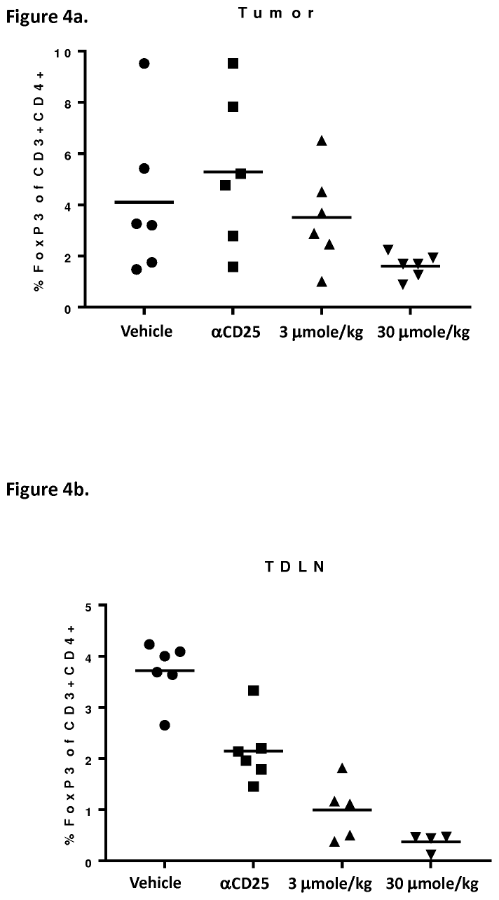

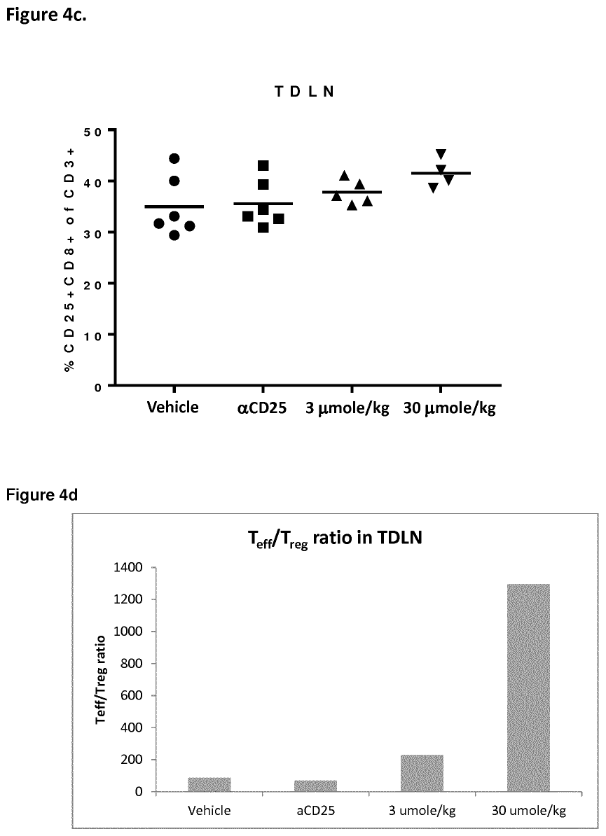

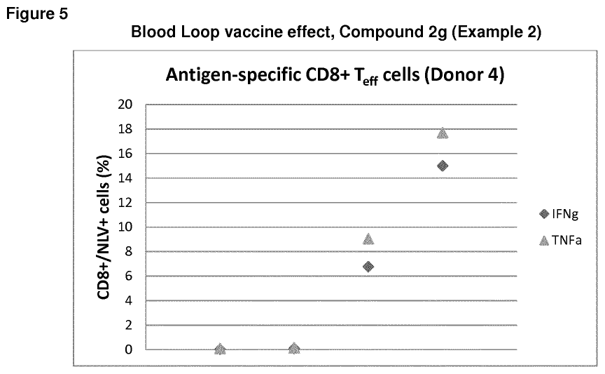

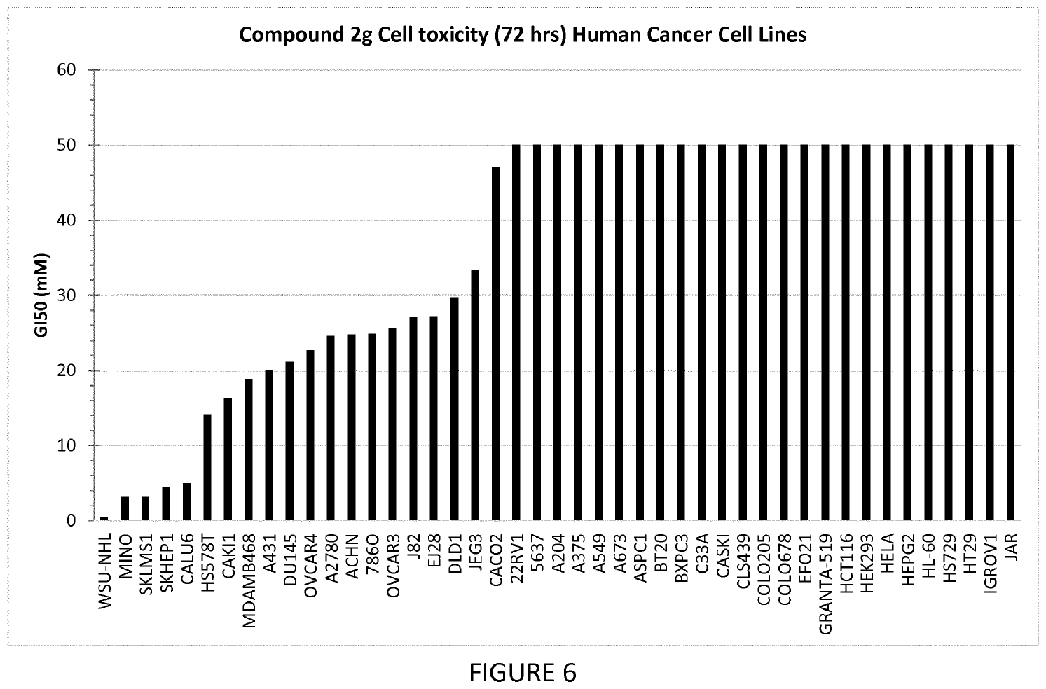

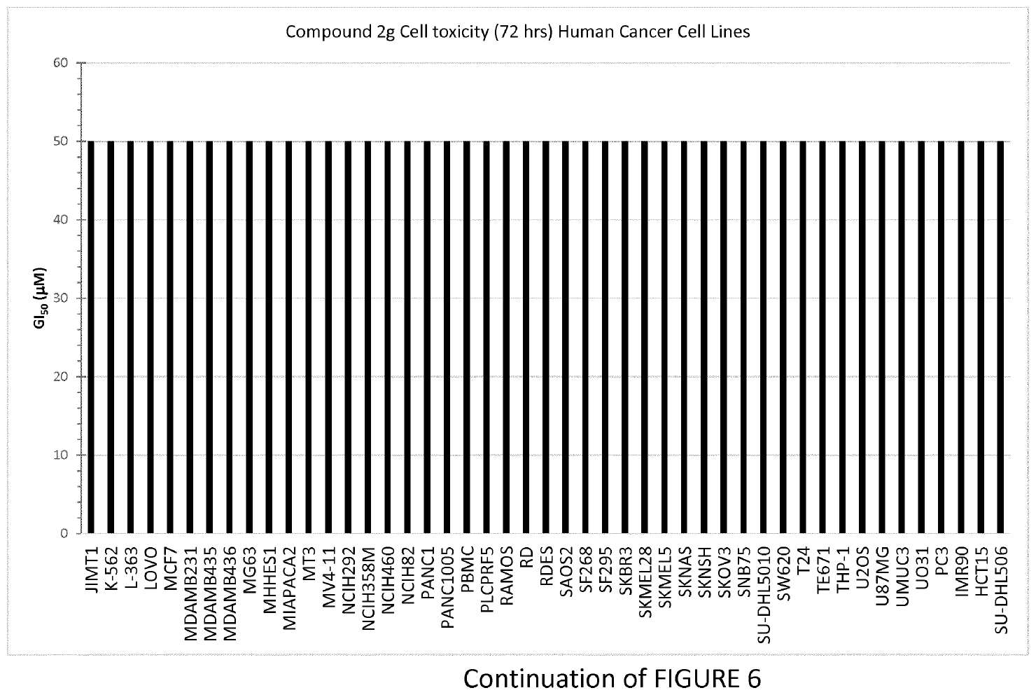

FIG. 4 shows the percent FOXP3 positive cells out of all CD3/CD4 double positive cells in (a) tumour and (b) tumour-draining lymph nodes. It can be observed that the percentage of FOXP3 positive cells (T_{reg}) is reduced, in a dose-dependent manner, in both tumour and TDLN, after MALT1 inhibitor therapy. FIG. 4c shows that the amount of CD8/CD25 double positive cells (T_{eff}) is not reduced in the TDLN derived from the same samples as in 4b. This suggests that MALT1 inhibitor is selectively inhibiting T_{reg} cells, not T_{eff} cells or affecting T-cells in general. FIG. 4d shows that the ratio between T_{eff} cells (CD8+, CD25+) and T_{reg} cells (CD25+FOXP3+) is dramatically changed by MALT1 inhibitor therapy (for four days). Most pronounced change of the T_{reg}/T_{eff} ratio was observed with 30 μmole/kg. It can be concluded that MALT1 inhibitor treatment in vivo, in the syngeneic MB49 bladder cancer model, reduces T_{reg} cell infiltration in the tumour and tumour-draining lymph nodes while at the same time maintaining high T_{eff} cell levels, thus changing the T_{eff}/T_{reg} cell ratio in a favourable way.

### Biology Example 5

Impact on T-Effector Cells in Human Whole Blood Stimulated with CMV-Vaccine

Freshly drawn whole blood from healthy volunteers (2 mL) in a tube was incubated under rotation at 37° C. for 6 hours. Directly after blood sampling, vehicle, 4 μM compound 2g of Example 2, 120 nM of a peptide conjugate, minimal tetanus toxin epitope (MTTE)₃-CMV or combinations thereof were added. After 2 hours Brefeldin-A was added to allow intra-cellular analysis of cytokines (IFNγ and TNFα) by flow-cytometry. Antigen-specific CD8+ T-cells were identified by fluorescence-conjugated tetrameric complexes of HLA-A2 molecules loaded with the NLV peptide epitope (NLVPMVATV) of CMV pp65.

FIG. 5 shows the enhancing effect of MALT1 inhibition on CMV-specific (NLV+) CD8+T_{eff} cells expressing IFNγ or TNFα in whole blood three healthy blood donors treated with (1) vehicle, (2) 4 μM of compound 2g of Example 2, (3) 120 nM of (MTTE)3-CMV, and (4) 120 nM of (MTTE)₃-CMV+4 μM of compound 2g Example 2. In line with the inhibition of T_{reg} differentiation and function by MALT1 inhibitor (FIGS. 1 and 2) the whole blood loop assay demonstrated that MALT1 inhibition caused an increase in the percentage of activated antigen-specific IFNγ+ and TNFα+CD8+ T cells stimulated by the (MTTE)₃-CMV vaccine. This shows that in a complex, vaccine-induced, immune-response, a MALT1 inhibitor could enhance the response and shift the balance towards an antigen-specific T_{eff} dominated response.

### Biology Example 5

A broad panel of human cancer cell lines (81 cell lines from solid cancers and 12 cell lines from hematological tumours, and PBMC as reference) provided as a service by Oncolead GmbH & Co KG, Karlsfeld, Germany were treated with compound 2g (example 2) at 6 concentrations (50 μM to 0.05 nM) for 72 hours.

FIG. 6 depicts the GI₅₀ across the cell line panel. Only one cell line, WSU-NHL, had a GI₅₀<1 μM This result was not possible to confirm with a follow-up 5 day cell growth assay using compound 2g and compound 3.

Table Cell growth WSU-NHL 5 days (CC₅₀)

Compound

| Cell Line | Example 2 (CC₅₀ μM) | Example 3 (CC₅₀ μM) |
|---|---|---|
| WSU-NHL | 77 | 31 |

FIG. 6 thus shows that even though MALT1 inhibitors are very potent (compound 2 g has an IC₅₀ less than 10 nM in Biology Example 1 above), the compound has essentially no antiproliferative effect on the majority of cell lines tested. Even the small handful of compounds with a GI₅₀ around the 20 uM level would not generally be regarded as viable anticancer agents, as in vivo concentrations at anything approaching this level are unlikely. The conclusion is thus that the antiproliferative activity of the compounds is not via direct inhibition of MALT1 in cancer cells, with the possible exception of the unusual cancer ABC DBCL, where the NFKB hypothesis put forward by Novartis may have merit.

### Aspects

The following aspects are included in the present disclosure.
1. A MALT1 inhibitor for use in the immuno-oncologic treatment of cancer, independently of dysregulated NFkB pathway activation within the cancer cells.
2. A MALT1 inhibitor for use in the immuno-oncologic treatment of cancer, wherein the tumoural tissue is characterized by infiltration of a) Fox P3 positive T_{reg} lymphocytes, and b) CD4+ and CD8+T_{eff} lymphocytes.
3. Use according to any of aspects 1-2, wherein the cancer is bladder cancer.
4. Use according to any of aspects 1-2, wherein the cancer is colon cancer.
5. Use according to any of aspects 1-2 wherein the cancer is hepatocellular cancer.
6. Use according to any of aspects 1-2, wherein the cancer is Small Cell or Non-Small Cell lung cancer.
7. Use according to any preceding aspect, wherein the MALT1 inhibitor is an RNA-based drug or a small molecule drug.
8. Use according to aspect 7, wherein the MALT1 inhibitor is a small molecule drug which is administered orally.
9. Use according to aspect 7 or 8, wherein the small molecule drug is selected from the formula I:

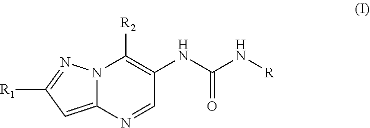

or a pharmaceutically acceptable salt thereof, wherein,
R, is halogen, cyano, or C₁-C₃ alkyl optionally substituted by halogen;
R₂ is C₁-C₆ alkyl optionally substituted one or more times by C₁-C₆ alkyl, C₂-C₆ alkenyl, hydroxyl, N,N-di-C₁-C₆alkylamino, N-mono-C₁-C₆alkylamino, O—Rg, Rg, phenyl, or by C₁-C₆ alkoxy wherein said alkoxy again may optionally be substituted by C₁-C₆ alkoxy, N,N-di-C₁-C₆alkylamino, Rg or phenyl; C₃-C₆cycloalkyl optionally substituted by C₁-C₆ alkyl, N,N-di-C₁-C₆ alkylamino or C₁-C₆ alkoxy-C₁-C₆ alkyl, and/or two of said optional substituents together with the atoms to which they are bound may form an annulated or spirocyclic 4-6 membered saturated heterocyclic ring comprising 1-2 O atoms; phenyl optionally substituted by C₁-C₆ alkoxy; a 5-6 membered heteroaryl ring having 1 to 3 heteroatoms selected from N and O said ring being optionally substituted by C₁-C₆ alkyl which may be optionally substituted by amino or hydroxy; Rg; or N,N-di-C₁-C₆C alkyl amino carbonyl; wherein
Rg is a 5-6 membered heterocyclic ring having 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is phenyl independently substituted two or more times by Ra, 2-pyridyl independently substituted one or more times by Rb, 3-pyridyl independently substituted one or more times by Rc, or 4-pyridyl independently substituted one or more times by Rd; wherein Ra independently from each other is halogen; cyano; —COOC₁-C₆alkyl; C₁-C₆ alkoxy; C₁-C₆ alkyl optionally substituted by halogen or a 5-6 membered heterocyclyl ring having 1 to 2 heteroatoms selected from N and O which ring is optionally substituted by C₁-C₆alkyl; a 5-6 membered heteroaryl ring having 1 to 3 heteroatoms selected from N and O said ring being optionally substituted by amino, C₁-C₆alkyl optionally substituted by amino or hydroxy, or by N-mono- or N,N-di-C₁-C₆C alkylamino carbonyl; and/or
two Ra together with the ring atoms to which they are bound may form a 5 to 6 membered heterocyclic or heteroaromatic ring having 1 to 2 N atoms, any such ring being optionally substituted by C₁-C₆ alkyl or oxo;
Rb, Rc and Rd independently from each other are halogen; oxo; hydroxy; cyano; C₁-C₆ alkoxy optionally substituted by halogen; C₁-C₆ alkoxy carbonyl; phenyl; N,N-di-C₁-C₆ alkyl amino; C₁-C₆alkyl optionally substituted by halogen or phenyl; a 5-6 membered heteroaryl ring having 1 to 3 N atoms said ring being optionally substituted by C₁-C₆ alkyl optionally substituted by amino or hydroxy, or by mono- or di-N—C₁-C₆alkylamino carbonyl; O—Rh; or Rh; wherein
Rh is a 5-6 membered heterocyclyl ring having 1 to 4 heteroatoms selected from N, O and S said ring being optionally substituted by C₁-C₆ alkyl, hydroxy or oxo.
10. Use according to aspect 9, wherein R₁ is halogen; R₂ is C₁-C₆alkyl optionally substituted one or more times by C₁-C₆ alkyl, C₂-C₆ alkenyl, hydroxy, N,N-di-C₁-C₆ alkyl amino, N-mono-C₁-C₆ alkyl amino, O—Rg, Rg, phenyl, or by C₁-C₆alkoxy, wherein said alkoxy again may optionally be substituted by C₁-C₆ alkoxy, N,N-di-C₁-C₆ alkylamino, Rg or phenyl; wherein
   Rg is a 5-6 membered heterocyclic ring containing 1-3 heteroatoms selected from N and O said ring being optionally substituted by C₁-C₆alkyl, C₁-C₆alkoxy-C₁-C₆alkyl, C₁-C₆ alkoxy-carbonyl;
   R is 2-pyridyl independently substituted one or more times by Rb, 3-pyridyl independently substituted one or more times by Rc, or 4-pyridyi independently substituted one or more times by Rd; and
   Rb, Rc and Rd are as defined in aspect 9;
   or a pharmaceutically acceptable salt thereof.
11. Use according to aspect 9 or 10, wherein R, is chloro, and the remaining substituents are as defined therein.
12. Use according to aspect 9, wherein R₁ is chloro; R is 2-pyridyl independently substituted one or more times by Rb; or R is 3-pyridyl independently substituted one or more times by Rc; or R is 4-pyridyl independently substituted one or more times by Rd; wherein Rb, Rc and
   Rd are as defined in aspect 9, and the remaining substituents are as defined in aspect 10.
13. Use according to aspect 9, wherein R, is halogen, cyano, or C₁-C₃ alkyl optionally substituted by halogen;
   R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkyl amino, O—Rg, Rg, phenyl, or by C₁-C₆ alkoxy wherein said alkoxy again may optionally be substituted by C₁-C₆ alkoxy, N,N-di-C₁-C₆ alkyl amino, Rg or phenyl; C₃-C₆cycloalkyl optionally substituted by C₁-C₆ alkyl, N,N-di-C₁-C₆alkyl amino or C₁-C₆ alkoxy-C₁-C₆alkyl, or two of said optional substituents together with the atoms to which they are bound may form an annulated or spirocyclic 4-6 membered saturated heterocyclic ring comprising 1-2 O atoms; phenyl optionally substituted by C₁-C₆ alkoxy; a 5-6 membered heteroaryl containing 1 to 3 heteroatoms selected from N and O optionally substituted by C₁-C₆ alkyl which may optionally be substituted by amino or hydroxy; Rg; or N,N-di-C₁-C₆ alkyl amino carbonyl; wherein
   Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
   R is phenyl independently substituted two or more times by Ra; wherein
   Ra independently from each other is halogen; cyano; —COOC₁-C₆alkyl; C₁-C₅ alkoxy; C₁-C₆ alkyl optionally substituted by halogen or a 5-6 membered heterocyclic ring containing 1 to 2 N atoms said ring being optionally substituted by C₁-C₆ alkyl; a 5-6 membered heteroaryl ring containing 1 to 3 N atoms said ring being optionally substituted by amino, C₁-C₆alkyl optionally substituted by amino or hydroxy, or by N-mono- or N,N-di-C₁-C₆ alkylamino carbonyl;
   and/or,
   two Ra together with the ring atoms to which they are bound form a 5 to 6 membered heterocyclic or heteroaromatic ring containing 1 to 2 N atoms, any such ring being optionally substituted by C₁-C₆ alkyl or oxo.
14. Use according to aspect 9 or 13, wherein R, is methyl.
15. Use according to aspect 9 or 13, wherein
   R₁ is halogen;
   R is phenyl independently substituted two or more times by Ra; wherein
   Ra independently from each other is halogen; cyano; —COOC₁-C₆ alkyl; C₁-C₆ alkoxy; C₁-C₆ alkyl optionally substituted by fluoro or a 5-8 membered heterocyclic ring containing 1 to 2 N atoms which heterocyclyl is optionally substituted by C₁-C₆ alkyl; a 5-6 membered heteroaryl ring containing 1 to 3 N atoms said ring being optionally substituted by amino, C₁-C₆ alkyl optionally substituted by amino or hydroxy, or by N-mono- or N,N-di-C₁-C₆ alkylamino carbonyl, and
   the remaining substituents are as defined in aspect 9.
16. Use according to aspect 9, wherein
   R₁ is fluoro;
   R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkylamino, O—Rg, Rg, phenyl, or by C₁-C₆ alkoxy, wherein said alkoxy again may optionally be substituted by C₁-C₆ alkoxy or Rg or phenyl; wherein
   Rg is a 5-6 membered heterocyclic ring having 1-3 heteroatoms selected from N and O said ring being optionally substituted by C₁-C₆ alkyl, C₁-C₆ alkoxy-C₁-C₆ alkyl, C₁-C₆ alkoxy-carbonyl;
   R is 2-pyridyl substituted one or more times by Rb; and
   Rb independently from each other is halogen; oxo; hydroxy; cyano; C₁-C₆ alkoxy optionally substituted by halogen; C₁-C₆ alkoxy carbonyl; phenyl; N,N-di-C₁-C₆ alkylamino; C₁-C₆ alkyl optionally substituted by halogen or phenyl; a 5-6 membered heteroaryl ring having 1 to 3 N atoms said ring being optionally substituted by C₁-C₆ alkyl optionally substituted by amino or hydroxy, or by mono- or di-N—C₁-C₆ alkylamino carbonyl; O—Rh; or Rh; wherein
   Rh is a 5-6 membered heterocyclyl ring having 1 to 4 heteroatoms selected from N, O and S said ring being optionally substituted by C₁-C₆ alkyl, hydroxy or oxo,
17. Use according to aspect 9, wherein
   R₁ is fluoro;
   R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkyl amino, O—Rg, Rg, phenyl, or by C₁-C₆ alkoxy, wherein said alkoxy again may optionally be substituted by C₁-C₆ alkoxy, N,N-di-C₁-C₆ alkyl amino, Rg or phenyl; wherein
   Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
   R is 3-pyridyl substituted one or more times by Rc; and
   Rc independently from each other is halogen; oxo; hydroxyl; cyano; C₁-C₆ alkoxy optionally substituted by halogen; C₁-C₆ alkoxy carbonyl; phenyl; N,N-di-C₁-C₆ alkyl amino; C₁-C₆ alkyl optionally substituted by halogen or phenyl; a 5-6 membered heteroaryl ring having 1 to 3 N atoms said ring being optionally substituted by C₁-C₆ alkyl optionally substituted by amino or hydroxy, or by mono- or di-N—C₁-C₆ alkylamino carbonyl; O—Rh; or Rh; wherein
   Rh is a 5-6 membered heterocyclyl having 1 to 4 heteroatoms selected from N, O and S said ring being optionally substituted by C₁-C₆ alkyl, hydroxyl or oxo.
18. Use according to aspect 9, wherein
   R₁ is fluoro;
   R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkyl amino, O—Rg, Rg, phenyl, or by C₁-C₆ alkoxy, wherein said alkoxy again may optionally be substituted by C₁-C₆ alkoxy, N,N-di-C₁-C₆ alkyl amino, Rg or phenyl; wherein
   Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
   R is 4-pyridyl substituted one or more times by Rd; and
   Rd independently from each other is halogen; oxo; hydroxyl; cyano; C₁-C₆ alkoxy optionally substituted by halogen; C₁-C₆ alkoxy carbonyl; phenyl; N,N-di-C₁-C₆ alkyl amino; C₁-C₆ alkyl optionally substituted by halogen or phenyl; a 5-6 membered heteroaryl ring containing 1 to 3 N atoms said ring being optionally substituted by C₁-C₆ alkyl optionally substituted by amino or hydroxy, or by mono- or di-N—C₁-C₆ alkylamino carbonyl; O—Rh; or Rh; wherein
   Rh is a 5-6 membered heterocyclyl containing 1 to 4 heteroatoms selected from N, O and S said ring being optionally substituted by C₁-C₆ alkyl, hydroxyl or oxo.
19. Use according to aspect 9, wherein the compound is selected from
(S)-1-(6-(2H-1,2,3-triazol-2-yl)-5-(trifluoromethyl)pyridin-3-yl)-3-(2-chloro-7-(1-methoxy ethyl) pyrazolo[1,5-a]pyrimidin-6-yl)urea;
(S)-1-(2-chloro-7-(1-methoxyethyl)pyrazolo[1,5-a]pyrimidin-6-yl)-3-(2-(trifluoromethyl)pyridin-4-yl)urea;
(S)-1-(2-chloro-7-(1-methoxyethyl)pyrazolo[1,5-a]pyrimidin-6-yl)-3-(1-methyl-2-oxo-5-(trifluoromethyl)-1,2-dihydropyridin-3-yl)urea;
(R)-1-(6-(2H-1,2,3-triazol-2-yl)-5-(trifluoromethyl)pyridin-3-yl)-3-(2-chloro-7-(1-methoxy\hat{ }2-methylpropyl)pyrazolo[1,5-a]pyrimidin-6-yl)urea;
(R)-1-(5-chloro-6-(2H-1,2,3-triazol-2-yl)pyridin-3-yl)-3-(2-chloro-7-(1-methoxy-2-methyl propyl) pyrazolo[1,5-a]pyrimidin-6-yl)urea;
(S)-1-(7-(1-methoxyethyl)-2-methylpyrazolo[1,5-a]pyrimidin-6-yl)-3-(2-(trifluoromethyi)pyridin-4-yl)urea;
(S)-1-(2-fluoro-7-(1-methoxyethyl)pyrazolo[1,5-a]pyrimidin-6-yl)-3-(2-(trifluoromethyl)pyridin-4-yl)urea; (S)-1-(2-chlQro-7-(1-methQxyeihyl)p\hat{ }urea;
(S)-1-(5-chloro-6-methoxypyridin-3-yl)-3-(2-chloro-7-(1-methoxyethyl)pyrazolo[1,5-a]pyrimidin-6-yl)urea;
(S)-1-(5-chloro-6-(2H-1,2,3-triazol-2-yl)pyridin-3-yl)-3-(2-chloro-7-(1-methoxyethyl) pyrazolo[1,5-a]pyrimidin-6-yl)urea;
(S)-1-(2-chloro-7-(1-methoxyethyl)pyrazolo[1,5-a]pyrimidin-6-yl)-3-(5-methoxy-6-(2H-1,2,3-triazol-2-yl)pyridin-3-yl)urea;
(S)-1-(2-chloro-7-(1-methoxyethyl)pyrazolo[1,5-a]pyrimidin-6-yl)-3-(2-chloropyridin-4-yl)urea;
(S)-methyl 3-chloro-5-(3-(2-chloro-7-(1-methoxyethyl)pyrazolo[1,5-a]pyrimidin-6-yl) ureido) benzoate;
1-(5-chloro-6-(2H-1,2,3-triazol-2-yl)pyridin-3-yl)-3-(2-chloro-7-(2-methoxypropan-2-yl) pyrazolo[1,5-a]pyrimidin-6-yl)urea;
1-(2-chloro-7-(2-methoxypropan-2-yl)pyrazolo[1,5-a]pyrimidin-6-yl)-3-(2-(trifluoromethyl) pyridin-4-yl)urea;
1-(5-chloro-6-(2H-1,2,3-triazol-2-yl)pyridin-3-yl)-3-(2-chloro-7-(1-methylcyclopropyl) pyrazolo[1,5-a]pyrimidin-6-yl)urea;
1-(2-chloro-7-(1-methylcyclopropyl)pyrazolo[1,5-a3pyrimidin-6-yl)-3-(2-(trifluoromethyl) pyridin-4-yl)urea;
1-(5-chloro-6-methoxypyridin-3-yl)-3-(2-chloro-7-isopropylpyrazolo[1,5-a]pyrimidin-6-yl) urea;
1-(2-chloro-7-isopropylpyrazolo[1,5-a]pyrimidin-6-yl)-3-(3-cyano-4-(3-methyl-1H-1,2,4-triazol-1-yl)phenyl)urea;
1-(3-chloro-4-(2H-1,2,3-triazol-2-yl)phenyl)-3-(2-chloro-7-isopropylpyrazolo[1,5-a]pyrimidin-6-yl)urea;
1-(5-chloro-6-(2H-1,2,3-triazol-2-yl)pyridin-3-yl)-3-(2-chloro-7-isopropylpyrazolo[1,5-a]pyrimidin-6-yl)urea;
1-(5-chloro-6-(4-methyl-2H-1,2,3-triazol-2-yl)pyridin-3-yl)-3-(2-chloro-7-isopropylpyrazolo[1,5-a]pyrimidin-6-yl)urea;
1-(4-(2-aminopyrimidin-4-yl)-3-chlorophenyl)-3-(2-chloro-7-isopropylpyrazolo[1,5-a]pyrimidin-6-yl)urea;
1-(5-chloro-1-methyl-6-oxo-2-(1H-pyrazol-1-yl)-1,6-dihydropyridin-3-yl)-3-(2-chloro-7-isopropylpyrazolo[1,5-a]pyrimidin-6-yl)urea;
1-(5-chloro-6-ethoxypyridin-3-yl)-3-(2-chloro-7-isopropylpyrazolo[1,5-a]pyrimidin-6-yl)urea;
1-(5-bromopyridin-3-yl)-3-(2-chloro-7-isopropylpyrazolo[1,5-a]pyrimidin-6-yl)urea;
1-(2-chloro-7-isopropylpyrazolo[1,5-a]pyrimidin-6-yl)-3-(6-(1,1-dioxidoisothiazolidin-2-yl)-5-(trifluoromethyl)pyridin-3-yl)urea;
1-(3-chloro-4-(3-(hydroxymethyl)-5-methyl-1H-pyrazol-1-yl)phenyl)-3-(2-chloro-7-iso-propyl pyrazolo[1,5-a]pyrimidin-6-yl)urea;
1-(5-chloro-6-(1-methyl-1H-pyrazol-5-yl)pyridin-3-yl)-3-(2-chloro-7-isopropylpyrazolo[1,5-a]pyrimidin-6-yl)urea;
1-(2-chloro-7-isopropylpyrazolo[1,5-a]pyrimidin-6-yl)-3-(3,5-dichloro-4-(2H-1,2,3-triazol-2-yl)phenyl)urea;
1-(5-chloro-2-oxoindolin-7-yl)-3-(2-chloro-7-isopropylpyrazoio[1ᵢ5-a]pyrimidin-6-yl)urea;
1-(2-chloro-7-isopropylpyrazolo[1,5-a]pyrimidin-6-yl)-3-(1-methyl-2-oxo-5-(trifluoro-methyl)-1,2-dihydropyridin-3-yl)urea;
1-(5-chloro-2-((1-methylpyrrolidin-3-yl)oxy)-6-(2H-1,2,3-triazol-2-yl)pyridin-3-yl)-3-(2-chloro-7-isopropylpyrazolo[1,5-a]pyrimidin-6-yl)urea;
1-(7-(tert-butyl)-2-chloropyrazolo[1,5-a]pyrimidin-6-yl)-3-(5-chloro-6-(2H-1,2,3-triazol-2-yl)pyridin-3-yl)urea;
1-(7-(sec-butyl)-2-chloropyrazolo[1,5-a]pyrimidin-6-yl)-3-(5-chloro-6-(2H-1,2,3-triazol-2-yl)pyridin-3-yl)urea;
1-(2-chloro-7-(2-methyltetrahydrofuran-2-yl)pyrazolo[1,5-a]pyrimidin-6-yl)-3-(2-(trifluoro-methyl)pyridin-4-yl)urea;
(R)-1-(2-chloro-7-(1-methoxy-2-methylpropyl)pyrazolo[1,5-a]pyrimidin-6-yl)-3-(2-(trifluoro-methyl)pyridin-4-yl)urea;
1-(5-chloro-6-(2H-1,2,3-triazol-2-yl)pyridin-3-yl)-3-(2-chloro-7-cyclobutylpyrazolo[1,5-a]-pyrimidin-6-yl)urea;
(S)-1-(5-chloro-6-(2H-1,2,3-triazol-2-yl)pyridin-3-yl)-3-(2-chloro-7-(1-(2-methoxy-ethoxy)-ethyl)pyrazolo[1,5-a]pyrimidin-6-yl)urea;
(S)-1-(5-chloro-6-methoxypyridin-3-yl)-3-(2-chloro-7-(1-(2-methoxyethoxy)-ethyl)-pyrazolo-[1,5-a]pyrimidin-6-yl)urea;
(S)-1-(2-chloro-7-(1-(2-methoxyethoxy)ethyl) pyrazolo[1,5-a]pyrimidin-6-yl)-3-(2-(trifluoromethyl)-pyridin-4-yl)urea;
(R)-1-(5-chloro-6-(2H-1,2,3-triazol-2-yl)pyridin-3-yl)-3-(2-chloro-7-(1-(2-methoxy-ethoxy)-ethyl)pyrazolo[1,5-a]pyrimidin-6-yl)urea;
1-(5-chloro-6-(2H-1,2,3-triazol-2-yl)pyridin-3-yl)-3-(2-chloro-7-(1,4-dioxan-2-yl)-pyrazolo[1,5-a]pyrimidin-6-yl) urea;
(S)-1-(5-chloro-6-(2H-1,2,3-triazol-2-yl)pyridin-3-yl)-3-(2-chloro-7-(1-methoxypropan-2-yl)-pyrazolo[1,5-a]pyrimidin-6-yl)urea;
(R)-1-(5-chloro-8-(2H-1,2,3 triazol-2-yl)pyridin-3-yl)-3-(2-chloro-7-(1-methoxypropan-2-yl)-pyrazolo[1,5-a]pyrimidin-6-yl)urea;
(R)-1-(5-chloro-6-(2H-1,2,3-triazol-2-yl)pyridin-3-yl)-3-(2-chloro-7-(1-methoxyethyl)-pyrazolo[1,5-a]pyrimidin-6-yl) urea;
(R)-1-(2-chloro-7-(1-methoxyethyl)pyrazolo[1,5-a]pyrimidin-6-yl)-3-(2-(trifluoromethyl)-pyridin-4-yl)urea;
(R)-1-(2-chloro-7-(1-methoxyethyl)pyrazolo[1,5-a]pyrimidin-6-yl)-3-(1-methyl-2-oxo-5-(trifluoromethyl)-1,2-dihydropyridin-3-yl)urea;
1-(5-chloro-6-(2H-1,2,3-triazol-2-yl)pyridin-3-yl)-3-(2-chloro-7-(methoxy(phenyl)methyl)-pyrazolo[1,5-a]pyrimidin-6-yl)urea;
1-(5-chloro-6-(2H-1,2,3-triazol-2-yl)pyridin-3-yl)-3-(2-chloro-7-(1-(methoxymethyl) cyclobutyl)pyrazolo[1,5-a]pyrimidin-6-yl)urea;
1-(2-chloro-7-(1-(methoxymethyl)cyclobutyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-cyclopropylpyrazolo[1,5-a]pyrimidin-6-yl)urea;
1-(5-chloro-6-(2H-1,2,3-triazol-2-yl)pyridin-3-yl)-3-(2-chloro-7-(2-methoxyphenyl)pyrazolo[1,5-a]pyrimidin-6-yl)urea;
1-(6-(2H-1,2,3-triazoj-2-yl)-5-(trifluoromethyl)pyridine-3-y)-3-(2-chloro-7-(tetrahydrofuran-2-yl)pyrazolo[1,5-a]pyrimidin-6-yl)urea;
1-(5-chloro-6-(2H-1,2,3-triazol-2-yl)pyridin-3-yl)-3-(2-chloro-7-(4-methyltetrahydro-2H-pyran-4-yl)pyrazolo[1,5-a]pyrimidin-6-yl)urea;
1-(5-chloro-6-(2H-1,2,3-triazol-2-yl)pyridin-3-yl)-3-(2-chloro-7-(1,2-dimethoxyethyl) pyrazolo[1,5-a]pyrimidin-6-yl)urea;
1-(5-chloro-6-(2H-1,2,3-triazol-2-yl)pyridin-3-yl)-3-(2-chloro-7-(tetrahydrofuran-3-y) pyrazolo[1,5-a]pyrimidin-6-yl)urea;
1-(5-chloro-6-(2H-1,2,3-triazol-2-yl)pyridin-3-yl)-3-(2-chloro-7-(tetrahydro-2H-pyran-4-y) pyrazolo[1,5-a]pyrimidin-6-yl) urea;
1-(5-chloro-6-(2H-1,2,3-triazol-2-yl)pyridin-3-yl)-3-(2-chloro-7-(methoxymethyl) pyrazolo[1,5-a]pyrimidin-6-yl)urea;
1-(5-chloro-6-(2H-1,2,3-triazol-2-yl)pyridin-3-yl)-3-(2-chloro-7-((tetrahydro-2H-pyran-4-yl)methyl)pyrazolo[1,5-a]pyrimidin-6-yl)urea;
1-(5-chloro-6-(2H-1,2,3-triazol-2-yl)pyridin-3-yl)-3-(2-chloro-7-(isopropoxymethyl) pyrazolo[1,5-a]pyrimidin-6-yl)urea;
1-(5-chloro-6-(2H-1,2,3-triazol-2-yl)pyridin-3-yl)-3-(2-chloro-7-methylpyrazolo[1,5-a]pyrimidin-6-yl)urea;
1-(6-(2H-1,2,3-triazol-2-yl)-5-(trifluoromethyl)pyridin-3-yl)-3-(2-chloro-7-(furan-2-y) pyrazolo[1,5-a]pyrimidin-6-yl)urea;
1-(5-chloro-6-(2H-1,2,3-triazol-2-yl)pyridin-3-yl)-3-(2-chloro-7-(1,3-dimethoxypropyl) pyrazolo[1,5-a]pyrimidin-6-yl)urea;
(R)-1-(6-(2H-1,2,3-triazol-2-yl)-5-(trifluoromethyl)pyridin-3-yl)-3-(7-(1-(benzyloxy)ethyl)-2-chloropyrazolo[1,5-a]pyrimidin-6-yl)urea;
tert-butyl 2-(2-chloro-6-(3-(5-chloro-6-(2H-1,2,3-triazol-2-yl)pyridin-3-yl)ureido)pyrazolo[1,5-a]pyrimidin-7-yl)morpholine-4-carboxylate;
1-(7-(3-oxabicyclo[3.1.0]hexan-6-yl)-2-chloropyrazolo[1,5-a]pyrimidin-6-yl)-3-(5-chloro-6-methoxypyridin-3-yl)urea;
1-(5-chloro-6-(2H-1,2,3-triazol-2-yl)pyridin-3-y)-3-(2-chloro-7-(5-oxaspiro[2.4]heptan-1-yl) pyrazolo[1,5-a]pyrimidin-6-yl)urea;
1-(5-chloro-6-methoxypyridin-3-yl)-3-(2-chloro-7-(pyridin-4-yl)pyrazolo[1,5-a]pyrimidin-6-yl)urea;
2-chloro-6-(3-(5-chloro-6-(2H-1,2,3-triazol-2-yl)pyridin-3-yl)ureido)-N,N-dimethyl pyrazolo[1,5-a]pyrimidine-7-carboxamide;
(S)-1-(5-chloro-6-(2H-1,2,3-triazol-2-yl)pyridin-3-yl)-3-(7-(1-methoxyethyl)-2-methy pyrazolo[1,5-a]pyrimidin-6-yl)urea;
1-(5-chloro-6-(2H-1,2,3-triazol-2-yl)pyridin-3-yl)-3-(2-methyl-7-(1-methylcyclopropyl) pyrazolo[1,5-a]pyrimidin-6-yl)urea;
1-(5-chloro-6-methoxypyridin-3-yl)-3-(2-methyl-7-(1-methylcyclopropyl)pyrazolo[1,5-a]pyrimidin-6-yl)urea;
(S)-1-(5-chloro-6-(2H-1,2,3-triazol-2-yl)pyridin-3-yl)-3-(2-chloro-7-(1-methoxy-2-methylpropyl)pyrazolo[1,5-a]pyrimidin-6-yl)urea;
(S)-1-(2-chloro-7-(1-methoxy-2-methylpropyl)pyrazolo[1,5-a]pyrimidin-6-yl)-3-(2-(trifluoromethyl)pyridin-4-yl)urea;
1-(5-chloro-6-(2H-1,2,3-triazol-2-y)pyridin-3-y)-3-(2-chloro-7-(1-(methoxymethyl) cyclopropyl)pyrazolo[1,5-a]pyrimidin-6-yl)urea;
1-(2-chloro-7-(1-(methoxymethyl)cyclopropyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(7-(1-(methoxymethyl)cyclopropyl)-2-methylpyrazolo[1,5-a]pyrimidin-6-yl)urea;
1-(7-(1-(methoxymethyl)cyclopropyl)-2-methylpyrazolo[1,5-a]pyrimidin-6-yl)-3-(2-(trifluoromethyl)pyridin-4-yl)urea;
1-(2-chloro-7-(2-(tetrahydro-2H-pyran-4-yl)propan-2-yl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-(1,2-dimethoxypropan-2-yl)pyrazolo[1,5-a]pyrimidin-6-yl)urea;
(S)-1-(5-chloro-6-(2H-1,2,3-triazol-2-yl)pyridin-3-yl)-3-(2-chloro-7-(1-(2-(dimethylamino) ethoxy)ethyl)pyrazolo[1,5-a]pyrimidin-6-yl)urea;
1-(5-chloro-6-(2H-1,2,3-triazol-2-yl)pyridin-3-yl)-3-(2-chloro-7-((4-methylmorpholin-3-yl) methyl)pyrazolo[1,5-a]pyrimidin-6-yl)urea;
(S)-1-(5-chloro-6-(2H-1,2_{&gt;}3-triazol-2-yl)pyridin-3-yl)-3-(2-chloro-7-(1-methylpiperidin-2-yl) pyrazolo[1,5-a]pyrimidin-6-yl)urea;
1-(5-chloro-6-(2H-1,2,3-triazol-2-yl)pyridin-3-yl)-3-(2-chloro-7-((S)-1-((R)-2-methoxy-propoxy)ethyl)pyrazolo[1,5-a]pyrimidin-6-yl)urea;
1-(5-chloro-6-(2H-1,2,3-triazol-2-yl)pyridin-3-yl)-3-(2-chloro-7-(1-methyl-1H-imidazol-2-yl)pyrazolo[1,5-a]pyrimidin-6-yl)urea;
1-(5-chloro-6-methoxypyridin-3-yl)-3-(2-chloro-7-(5-methyltetrahydrofuran-2-yl)pyrazolo[1,5-a]pyrimidin-6-yl)urea;
1-(5-chloro-6-(2H-1,2,3-triazol-2-yl)pyridin-3-yl)-3-(2-chloro-7-(1-(dimethylamino) cyclopropyl) pyrazolo[1,5-a]pyrimidin-6-yl)urea;
(S)-1-(2-chloro-7-(1-methoxyethyl)pyrazolo[1,5-a]pyrimidin-6-yl)-3-(5-(difluoromethyl)-6-(2H-1,2,3-triazol-2-yl)pyridin-3-yl)urea;
1-(2-chloro-7-(methoxy(tetrahydro-2H-pyran-4-yl)methyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-(2-(methoxymethyl) tetrahydrofuran-2-yl)pyrazolo[1,5-a]pyrimidin-6-yl)urea;
(S)-1-(2-chloro-7-(1-methoxyethyl)pyrazolo[1,5-a]pyrimidin-6-yl)-3-(2-(difluoromethyl) pyridin-4-yl)urea;
(S)-1-(5-chloro-6-(difluoromethoxy)pyridin-3-yl)-3-(2-chloro-7-(1-methoxyethyl) pyrazolo[1,5-a]pyrimidin-6-yl)urea;
(S)-1-(6-(2H-1,2,3-triazol-2-yl)-5-(trifluoromethyl)pyridin-3-yl)-3-(2-fluoro-7-(1-methoxy-ethyl)pyrazolo[1,5-a]pyrimidin-6-yl)urea;
(S)-1-(5-chloro-6-(2H-1,2,3-triazol-2-yl)pyridin-3-yl)-3-(2-fluoro-7-(1-methoxyethyl) pyrazolo[1,5-a]pyrimidin-6-yl)urea;
(S)-1-(5-chloro-6-(2H-1,2,3-triazol-2-yl)pyridin-3-yl)-3-(2-cyano-7-(1-methoxy-ethyl) pyrazolo[1,5-a]pyrimidin-6-yl)urea;
(S)-1-(5-chloro-6-(diiluoromethoxy)pyridin-3-yl)-3-(2-chloro-7-(1-(2-methoxyethoxy) ethyl) pyrazolo[1,5-a]pyrimidin-6-yl)urea;
1-(5-chloro-6-(2H-1,2,3-triazol-2-yl)pyridin-3-yl)-3-(2-chloro-7-((1R,2R)-1,2-propyl) pyrazolo[1,5-a]pyrimidin-6-yl)urea;
(S)-1-(5-chloro-2-(2-(dimethylamino)ethoxy)pyridin-3-yl)-3-(2-chloro-7-(1-methoxy ethyl) pyrazolo[1,5-a]pyrimidin-6-yl)urea;
1-(5-chloro-6-(2H-1,2,3-triazo!-2-yl)pyridin-3-yl)-3-(2-chloro-7-((S)-1-(((furan-3-yl)oxy)ethyl)pyrazolo[1,5-a]pyrimidin-6-yl)urea;
1-(5-chloro-6-(2H-1,2,3-triazol-2-yl)pyridin-3-yl)-3-(2-chloro-7-((S)-1-(((S)-tetrahydro-furan-3-yl)oxy)ethyl)pyrazolo[1,5-a]pyrimidin-6-yl)urea;
1-(5-chloro-6-(2H-1,2,3 triazol-2-yl)pyridin-3-yl)-3-(2-chloro-7-((S)-1-(((S)-ietrahydro-furan-3-yl)methoxy)ethyl)pyrazolo[1,5-a]pyrimidin-6-yl)urea;
1-(5-chloro-6-(2H-1,2,3-triazol-2-yl)pyridin-3-yl)-3-(2-chloro-7-((S)-1-(((R)-tetrahydro-furan-3-yl)methoxy)ethyl)pyrazolo[1,5-a]pyrimidin-6-yl)urea;
1-(5-chloro-6-(2H-1,2,3-triazol-2-yl)pyridin-3-yl)-3-(2-methyl-7-(2-methyltetrahydrofuran-2-yl)pyrazolo[1,5-a]pyrimidin-6-yl)urea;
(S)-1-(2-chloro-7-(1-methoxyethyl)pyrazolo[1,5-a]pyrimidin-6-yl)-3-(2-cyanopyridin-4-yl)urea;
1-(2-chloro-7-(2-(meth oxymethyl)tetrahydrofuran-2-y)pyrazolo[1,5-a]pyrimidin-6-yl)-3-(5-(trifluoromethyl)pyridin-3-yl)urea;
1-(6-(2H-1,2,3-triazol-2-yl)-5-(trifluoromethyl)pyridin-3-yl)-3-(2-chloro-7-(1-(dimethyl-amino)cydopropyl)pyrazolo[1,5-a]pyrimidin-6-yl)urea;
1-(6-(2H-1,2,3-triazol-2-yl)-5-(trifluoromethyl)pyridin-3-yl)-3-(2-chloro-7-(1,2-dimethoxy-ethyl)pyrazolo[1,5-a]pyrimidin-6-yl)urea;
1-(2-chloro-7-(1,2-dimethoxyethyl)pyrazolo[1,5-a]pyrimidin-6-yl)-3-(2-(trifluoromethyl) pyridin-4-yl)urea;
(S)-1-(6-(2H-1,2,3-triazol-2-yl)-5-(trifluoromethyl)pyridin-3-yl)-3-(2-chloro-7-(1-methoxy-propan-2-yl)pyrazolo[1,5-a]pyrimidin-6-yl)urea;
(R)-1-(6-(2H-1,2,3-triazol-2-yl)-5-(trifluoromethyl)pyridin-3-yl)-3-(2-chloro-7-(1-methoxy-propan-2-yl)pyrazolo[1,5-a]pyrimidin-6-yl)urea;
1-(5-(2H-1,2,3-triazol-2-yl)-2-(trifluoromethyl)pyridin-4-yl)-3-(2-chloro-7 [1,5-a]pyrimidin-6-yl)urea;
(R)-1-(6-(2H-1,2,3-triazo-2-yl)-5-(trifluoromethyl)pyridyin-3-yl)-3-(2-chloro-7-(1-hydroxy-ethyl) pyrazolo[1,5-a]pyrimidin-6-yl)urea;
(S)-1-(2-chloro-7-(1-hydroxyethyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-(morpholin-2-yl)pyrazolo[1,5-a]pyrimidin-8-yl)urea;
1-(5-chloro-6-(2H-1,2,3-triazol-2-yl)pyridin-3-yl)-3-(2-chloro-7-(4-methylmorpholin-2-yl) pyrazolo[1,5-a]pyrimidin-6-yl)urea;
1-(4-(4-(aminomethyl)-1H-pyrazol-1-yl)-3-chlorophenyl)-3-(2-chloro-7-isopropyl pyrazolo[1,5-a]pyrimidin-6-yl)urea;
1-(6-(4-(aminomethyl)-1H-pyrazol-1-yl)-5-chloropyridin-3-yl)-3-(2-chloro-7-isopropyl pyrazolo[1,5-a]pyrimidin-6-yl)urea;
(S)-1-(5-chloro-6-(2H-1,2,3-triazol-2-yl)pyridin-3-yl)-3-(2-chloro-7-(1-(methylamino) ethyl) pyrazolo[1,5-a]pyrimidin-6-yl)urea;
2-(3-(2-chloro-7-isopropylpyrazolo[1,5-a]pyrimidin-6-yl)ureido)-4-(trifluoromethyl)pyridine 1-oxide;
(R)-1-(5-chloro-6-(2H-1,2,3-triazol-2-yl)pyridin-3-yl)-3-(2-chloro-7-(1-(dimethylamino) ethyl)pyrazolo[1,5-a]pyrimidin-6-yl)urea;
(S)-1-(5-chloro-6-(2H-1,2,3-triazol-2-yl)pyridin-3-yl)-3-(2-chloro-7-(1-(dimethylamino) ethyl)pyrazolo[1,5-a]pyrimidin-6-yl)urea;
1-(5-chloro-6-(2H-1,2,3-triazol-2-yl)pyridin-3-yl)-3-(2-chloro-7-(morpholin-3-yl) pyrazolo[1,5-a]pyrimidin-6-yl)urea;
(S)-1-(5-chloro-6-(2H-1,2,3-triazol-2-yl)pyridin-3-yl)-3-(2-chloro-7-(2-methyl-1-(methyl-amino) propyl)pyrazolo[1,5-a]pyrimidin-6-yl)urea;
1-(5-chloro-6-(2H-1,2,3-triazol-2-yl)pyridin-3-yl)-3-(2-chloro-7-(4-methylmorpholin-3-yl) pyrazolo[1,5-a]pyrimidin-6-yl)urea;
(R)-1-(5-chloro-6-(2H-1,2,3-triazol-2-yl)pyridin-3-yl)-3-(2-chloro-7-(1,2-dimethoxyethyl) pyrazolo[1,5-a]pyrimidin-6-yl)urea; and
(S)-1-(5-chloro-6-(2H-1,2,3-triazol-2-yl)pyridin-3-yl)-3-(2-chloro-7-(1,2-dimethoxyethyl) pyrazolo[1,5-a]pyrimidin-6-yl)urea.
20. Use of a MALT1 inhibitor in the treatment of cancer, in combination with a treatment regime comprising at least one further immuno-oncology agent.
21. Use according to aspect 20, wherein the tumoural tissue is characterized by infiltration of a) Fox P3 positive T_{reg} lymphocytes, and b) CD4+ and CD8+T_{eff} lymphocytes.
22. Use according to any of aspects 20-21, wherein the further immuno-oncology treatment regime is selected from antibodies, cytokine therapy, adoptive T-cell therapy and immunostimulatory polysaccharides.
23. Use according to aspect 23, wherein the antibody is a checkpoint antibody.
24. Use according to aspect 23, wherein the checkpoint antibody is a PD1 inhibitor, such as or BGB-A317, and more preferably nivolumab or pembrolizumab.
25. Use according to aspect 23, wherein the checkpoint antibody is a PD-L1 antibody, preferably atezolizemab, avelumab or durvalumab.
26. Use according to aspect 23, wherein the antibody is:
   an anti-CD52 antibody such as alemtuzumab;
   a CTLA4 antibody such as ipilimumab;
   a CD20 antibody such as ofatumumab or rituximab;
   an anti-4-1BB (CD137) antibody, such as Utomilumab;
   a GITR antibody;
   an anti-OX40 (CD134) antibody;
   an anti-CD40 antibody, such as Dacetuzumab or Lucatumumab
27. Use according to aspect 22, wherein the cytokine therapy comprises an interferon selected from IFNα, IFNβ, IFNγ and IFNλ, or an interleukin, preferably IL-2.
28. Use according to any of aspects 20-27, wherein the MALT1 inhibitor is an orally administered small molecule inhibitor and the further immuno-oncology treatment regime is administered parenterally, for example intravenously, intraperitoneally or as a depot.
29. Use according to any of aspects 20-28, wherein the MALT1 inhibitor is as defined in any of aspects 9-19.
30. A method for screening for small molecule drugs suitable for use in the immuno-oncologic treatment of cancer independently of dysregulated NFKB pathway activation, and/or cancers whose tumoural tissue is characterized by infiltration of a) Fox P3 positive T_{reg} lymphocytes, and b) CD4+ and CD8+T_{eff} lymphocytes in patients, the method comprising the steps of
   a) obtaining purified or recombinant MALT1
   b) contacting the isolated MALT1 with one or more putative drugs, and
   c) selecting those putative drugs that demonstrate a reduction in MALT1 activity.

1. A method for the prevention or treatment of cancer in a subject, the method comprising administering to said subject a mucosa associated lymphoid tissue lymphoma translocation protein 1 (MALT1) inhibitor as an immunomodulatory agent.

2. The method according to claim 1, wherein said cancer is not characterised by dysregulation of the NF-κB pathway in cancerous cells.

3. The method according to claim 1, wherein said cancer is characterised by the presence of both infiltrating regulatory T cells (Treg cells) and infiltrating effector T cells (Teff cells) in the tumour.

4. The method according to claim 1, wherein said cancer is prostate cancer, brain cancer, breast cancer, colon cancer, colorectal cancer, pancreatic cancer, hepatocellular cancer, ovarian cancer, lung cancer, cervical cancer, liver cancer, head/neck/throat cancer, skin cancer, bladder cancer or a hematologic cancer, preferably wherein said cancer is bladder cancer, colon cancer, hepatocellular cancer, or Small Cell or Non-Small Cell lung cancer.

5. The method according to claim 1, wherein said MALT1 inhibitor is a small molecule inhibitor, an RNA-based inhibitor, or a substrate analogue inhibitor, optionally wherein the method comprises oral administration of the MALT1 inhibitor.

6. The method according to claim 1, wherein said MALT1 inhibitor is a small molecule of formula I: 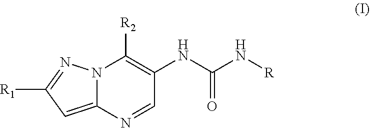
or a pharmaceutically acceptable salt thereof, wherein,
R₁ is halogen, cyano, or C₁-C₃ alkyl optionally substituted by halogen;
R₂ is C₁-C₆ alkyl optionally substituted one or more times by C₁-C₆ alkyl, C₂-C₆ alkenyl, hydroxyl, N,N-di-C₁-C₆ alkylamino, N-mono-C₁-C₆ alkylamino, O—Rg, Rg, phenyl, or by C₁-C₆ alkoxy wherein said alkoxy again may optionally be substituted by C₁-C₆ alkoxy, N,N-di-C₁-C₆ alkylamino, Rg or phenyl; C₃-C₆ cycloalkyl optionally substituted by C₁-C₆ alkyl, N,N-di-C₁-C₆ alkylamino or C₁-C₆ alkoxy-C₁-C₆ alkyl, and/or two of said optional substituents together with the atoms to which they are bound may form an annulated or spirocyclic 4-6 membered saturated heterocyclic ring comprising 1-2 O atoms; phenyl optionally substituted by C₁-C₆ alkoxy; a 5-6 membered heteroaryl ring having 1 to 3 heteroatoms selected from N and O said ring being optionally substituted by C₁-C₆ alkyl which may be optionally substituted by amino or hydroxy; Rg; or N,N-di-C₁-C₆ alkyl amino carbonyl; wherein
Rg is a 5-6 membered heterocyclic ring having 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is phenyl independently substituted two or more times by Ra, 2-pyridyl independently substituted one or more times by Rb, 3-pyridyl independently substituted one or more times by Rc, or 4-pyridyl independently substituted one or more times by Rd; wherein Ra independently from each other is halogen; cyano; —COOC₁-C₆ alkyl; C₁-C₆ alkoxy; C₁-C₆ alkyl optionally substituted by halogen or a 5-6 membered heterocyclyl ring having 1 to 2 heteroatoms selected from N and O which ring is optionally substituted by C₁-C₆ alkyl; a 5-6 membered heteroaryl ring having 1 to 3 heteroatoms selected from N and O said ring being optionally substituted by amino, C₁-C₆ alkyl optionally substituted by amino or hydroxy, or by N-mono- or N,N-di-C₁-C₆ alkylamino carbonyl; and/or
two Ra together with the ring atoms to which they are bound may form a 5 to 6 membered heterocyclic or heteroaromatic ring having 1 to 2 N atoms, any such ring being optionally substituted by C₁-C₆ alkyl or oxo;
Rb, Rc and Rd independently from each other are halogen; oxo; hydroxy; cyano; C₁-C₆ alkoxy optionally substituted by halogen; C₁-C₆ alkoxy carbonyl; phenyl; N,N-di-C₁-C₆ alkyl amino; C₁-C₆ alkyl optionally substituted by halogen or phenyl; a 5-6 membered heteroaryl ring having 1 to 3 N atoms said ring being optionally substituted by C₁-C₆ alkyl optionally substituted by amino or hydroxy, or by mono- or di-N—C₁-C₆ alkylamino carbonyl; O—Rh; or Rh; wherein
Rh is a 5-6 membered heterocyclyl ring having 1 to 4 heteroatoms selected from N, O and S said ring being optionally substituted by C₁-C₆ alkyl, hydroxy or oxo.

7. The method according to claim 6, wherein in said MALT1 inhibitor or said salt thereof, R₁ is halogen; R₂ is C₁-C₆ alkyl optionally substituted one or more times by C₁-C₆ alkyl, C₂-C₆ alkenyl, hydroxy, N,N-di-C₁-C₆ alkyl amino, N-mono-C₁-C₆ alkyl amino, O—Rg, Rg, phenyl, or by C₁-C₆ alkoxy, wherein said alkoxy again may optionally be substituted by C₁-C₆ alkoxy, N,N-di-C₁-C₆ alkylamino, Rg or phenyl; wherein
Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is 2-pyridyl independently substituted one or more times by Rb, 3-pyridyl independently substituted one or more times by Rc, or 4-pyridyi independently substituted one or more times by Rd; and
Rb, Rc and Rd are as defined in claim 9.

8. The method according to claim 6, wherein in said MALT1 inhibitor or said salt thereof, R₁ is chloro, and the remaining substituents are as defined therein.

9. The method according to claim 6, wherein in said MALT1 inhibitor or said salt thereof, R₁ is chloro; R is 2-pyridyl independently substituted one or more times by Rb; or R is 3-pyridyl independently substituted one or more times by Rc; or R is 4-pyridyl independently substituted one or more times by Rd; wherein Rb, Rc and Rd are as defined in claim 6, and the remaining substituents are as defined in claim 7.

10. The method according to claim 6, wherein in said MALT1 inhibitor or said salt thereof, R₁ is halogen, cyano, or C₁-C₃ alkyl optionally substituted by halogen;
R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkyl amino, O—Rg, Rg, phenyl, or by C₁-C₆ alkoxy wherein said alkoxy again may optionally be substituted by C₁-C₆ alkoxy, N,N-di-C₁-C₆ alkyl amino, Rg or phenyl; C₃-C₆ cycloalkyl optionally substituted by C₁-C₆ alkyl, N,N-di-C₁-C₆ alkyl amino or C₁-C₆ alkoxy-C₁-C₆ alkyl, or two of said optional substituents together with the atoms to which they are bound may form an annulated or spirocyclic 4-6 membered saturated heterocyclic ring comprising 1-2 O atoms; phenyl optionally substituted by C₁-C₆ alkoxy; a 5-6 membered heteroaryl containing 1 to 3 heteroatoms selected from N and O optionally substituted by C₁-C₆ alkyl which may optionally be substituted by amino or hydroxy; Rg; or N,N-di-C₁-C₆ alkyl amino carbonyl;
wherein
Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is phenyl independently substituted two or more times by Ra; wherein
Ra independently from each other is halogen; cyano; —COOC₁-C₆ alkyl; C₁-C₅ alkoxy; C₁-C₆ alkyl optionally substituted by halogen or a 5-6 membered heterocyclic ring containing 1 to 2 N atoms said ring being optionally substituted by C₁-C₆ alkyl; a 5-6 membered heteroaryl ring containing 1 to 3 N atoms said ring being optionally substituted by amino, C₁-C₆ alkyl optionally substituted by amino or hydroxy, or by N-mono- or N,N-di-C₁-C₆ alkylamino carbonyl;
and/or,
two Ra together with the ring atoms to which they are bound form a 5 to 6 membered heterocyclic or heteroaromatic ring containing 1 to 2 N atoms, any such ring being optionally substituted by C₁-C₆ alkyl or oxo.

11. The method according to claim 6, wherein in said MALT1 inhibitor or said salt thereof, R₁ is methyl.

12. The method according to claim 6, wherein in said MALT1 inhibitor or said salt thereof, R₁ is halogen;
R is phenyl independently substituted two or more times by Ra; wherein
Ra independently from each other is halogen; cyano; —COOC₁-C₆ alkyl; C₁-C₆ alkoxy; C₁-C₆ alkyl optionally substituted by fluoro or a 5-8 membered heterocyclic ring containing 1 to 2 N atoms which heterocyclyl is optionally substituted by C₁-C₆ alkyl; a 5-6 membered heteroaryl ring containing 1 to 3 N atoms said ring being optionally substituted by amino, C₁-C₆ alkyl optionally substituted by amino or hydroxy, or by N-mono- or N,N-di-C₁-C₆ alkylamino carbonyl, and
the remaining substituents are as defined in claim 6.

13. The method according to claim 6 wherein in said MALT1 inhibitor or said salt thereof, R₁ is fluoro;
R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkylamino, O—Rg, Rg, phenyl, or by C₁-C₆ alkoxy, wherein said alkoxy again may optionally be substituted by C₁-C₆ alkoxy or Rg or phenyl; wherein
Rg is a 5-6 membered heterocyclic ring having 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is 2-pyridyl substituted one or more times by Rb; and
Rb independently from each other is halogen; oxo; hydroxy; cyano; C₁-C₆ alkoxy optionally substituted by halogen; C₁-C₆ alkoxy carbonyl; phenyl; N,N-di-C₁-C₆ alkylamino; C₁-C₆ alkyl optionally substituted by halogen or phenyl; a 5-6 membered heteroaryl ring having 1 to 3 N atoms said ring being optionally substituted by C₁-C₆ alkyl optionally substituted by amino or hydroxy, or by mono- or di-N—C₁-C₆ alkylamino carbonyl; O—Rh; or Rh; wherein
Rh is a 5-6 membered heterocyclyl ring having 1 to 4 heteroatoms selected from N, O and S said ring being optionally substituted by C₁-C₆ alkyl, hydroxy or oxo,

14. The method according to claim 6 wherein in said MALT1 inhibitor or said salt thereof, R₁ is fluoro;
R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkyl amino, O—Rg, Rg, phenyl, or by C₁-C₆ alkoxy, wherein said alkoxy again may optionally be substituted by C₁-C₆ alkoxy, N,N-di-C₁-C₆ alkyl amino, Rg or phenyl; wherein
Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is 3-pyridyl substituted one or more times by Rc; and
Rc independently from each other is halogen; oxo; hydroxyl; cyano; C₁-C₆ alkoxy optionally substituted by halogen; C₁-C₆ alkoxy carbonyl; phenyl; N,N-di-C₁-C₆ alkyl amino; C₁-C₆ alkyl optionally substituted by halogen or phenyl; a 5-6 membered heteroaryl ring having 1 to 3 N atoms said ring being optionally substituted by C₁-C₆ alkyl optionally substituted by amino or hydroxy, or by mono- or di-N—C₁-C₆ alkylamino carbonyl; O—Rh; or Rh; wherein
Rh is a 5-6 membered heterocyclyl having 1 to 4 heteroatoms selected from N, O and S said ring being optionally substituted by C₁-C₆ alkyl, hydroxyl or oxo.

15. The method according to claim 6 wherein in said MALT1 inhibitor or said salt thereof, R₁ is fluoro;
R₂ is C₁-C₆ alkyl optionally substituted one or more times by C₁-C₆ alkyl, C₂-C₆ alkenyl, hydroxyl, N,N-di-C₁-C₆ alkyl amino, N-mono-C₁-C₆ alkyl amino, O—Rg, Rg, phenyl, or by C₁-C₆ alkoxy, wherein said alkoxy again may optionally be substituted by C₁-C₆ alkoxy, N,N-di-C₁-C₆ alkyl amino, Rg or phenyl; wherein
Rg is a 5-6 membered heterocyclic ring containing 1-3 heteroatoms selected from N and O said ring being optionally substituted by C₁-C₆ alkyl, C₁-C₆ alkoxy-C₁-C₆ alkyl, C₁-C₆ alkoxy-carbonyl;
R is 4-pyridyl substituted one or more times by Rd; and
Rd independently from each other is halogen; oxo; hydroxyl; cyano; C₁-C₆ alkoxy optionally substituted by halogen; C₁-C₆ alkoxy carbonyl; phenyl; N,N-di-C₁-C₆ alkyl amino; C₁-C₆ alkyl optionally substituted by halogen or phenyl; a 5-6 membered heteroaryl ring containing 1 to 3 N atoms said ring being optionally substituted by C₁-C₆ alkyl optionally substituted by amino or hydroxy, or by mono- or di-N—C₁-C₆ alkylamino carbonyl; O—Rh; or Rh; wherein
Rh is a 5-6 membered heterocyclyl containing 1 to 4 heteroatoms selected from N, O and S said ring being optionally substituted by C₁-C₆ alkyl, hydroxyl or oxo.

16. The method according to claim 6 wherein said MALT1 inhibitor is selected from:
(S)-1-(6-(2H-1,2,3-triazol-2-yl)-5-(trifluoromethyl)pyridin-3-yl)-3-(2-chloro-7-(1-methoxy ethyl) pyrazolo[1,5-a]pyrimidin-6-yl)urea;
(S)-1-(2-chloro-7-(1-methoxyethyl)pyrazolo[1,5-a]pyrimidin-6-yl)-3-(2-(trifluoromethyl) pyridin-4-yl)urea;
(S)-1-(2-chloro-7-(1-methoxyethyl)pyrazolo[1,5-a]pyrimidin-6-yl)-3-(1-methyl-2-oxo-5-(trifluoromethyl)-1,2-dihydropyridin-3-yl)urea;
(R)-1-(6-(2H-1,2,3-triazol-2-yl)-5-(trifluoromethyl)pyridin-3-yl)-3-(2-chloro-7-(1-methoxy\hat{ }2-methylpropyl)pyrazolo[1,5-a]pyrimidin-6-yl)urea;
(R)-1-(5-chloro-6-(2H-1,2,3-triazol-2-yl)pyridin-3-yl)-3-(2-chloro-7-(1-methoxy-2-methyl propyl) pyrazolo[1,5-a]pyrimidin-6-yl)urea;
(S)-1-(7-(1-methoxyethyl)-2-methylpyrazolo[1,5-a]pyrimidin-6-yl)-3-(2-(trifluoromethyi) pyridin-4-yl)urea;
(S)-1-(2-fluoro-7-(1-methoxyethyl)pyrazolo[1,5-a]pyrimidin-6-yl)-3-(2-(trifluoromethyl) pyridin-4-yl)urea; (S)-1-(2-chlQro-7-(1-methQxyeihyl)p\hat{ }urea;
(S)-1-(5-chloro-6-methoxypyridin-3-yl)-3-(2-chloro-7-(1-methoxyethyl)pyrazolo[1,5-a]pyrimidin-6-yl)urea;
(S)-1-(5-chloro-6-(2H-1,2,3-triazol-2-yl)pyridin-3-yl)-3-(2-chloro-7-(1-methoxyethyl) pyrazolo[1,5-a]pyrimidin-6-yl)urea;
(S)-1-(2-chloro-7-(1-methoxyethyl)pyrazolo[1,5-a]pyrimidin-6-yl)-3-(5-methoxy-6-(2H-1,2,3-triazol-2-yl)pyridin-3-yl)urea;
(S)-1-(2-chloro-7-(1-methoxyethyl)pyrazolo[1,5-a]pyrimidin-6-yl)-3-(2-chloropyridin-4-yl)urea;
(S)-methyl 3-chloro-5-(3-(2-chloro-7-(1-methoxyethyl)pyrazolo[1,5-a]pyrimidin-6-yl) ureido) benzoate;
1-(5-chloro-6-(2H-1,2,3-triazol-2-yl)pyridin-3-yl)-3-(2-chloro-7-(2-methoxypropan-2-yl) pyrazolo[1,5-a]pyrimidin-6-yl)urea;
1-(2-chloro-7-(2-methoxypropan-2-yl)pyrazolo[1,5-a]pyrimidin-6-yl)-3-(2-(trifluoromethyl) pyridin-4-yl)urea;
1-(5-chloro-6-(2H-1,2,3-triazol-2-yl)pyridin-3-yl)-3-(2-chloro-7-(1-methylcyclopropyl) pyrazolo[1,5-a]pyrimidin-6-yl)urea;
1-(2-chloro-7-(1-methylcyclopropyl)pyrazolo[1,5-a3 pyrimidin-6-yl)-3-(2-(trifluoromethyl) pyridin-4-yl)urea;
1-(5-chloro-6-methoxypyridin-3-yl)-3-(2-chloro-7-isopropylpyrazolo[1,5-a]pyrimidin-6-yl) urea;
1-(2-chloro-7-isopropylpyrazolo[1,5-a]pyrimidin-6-yl)-3-(3-cyano-4-(3-methyl-1H-1,2,4-triazol-1-yl)phenyl)urea;
1-(3-chloro-4-(2H-1,2,3-triazol-2-yl)phenyl)-3-(2-chloro-7-isopropylpyrazolo[1,5-a]pyrimidin-6-yl)urea;
1-(5-chloro-6-(2H-1,2,3-triazol-2-yl)pyridin-3-yl)-3-(2-chloro-7-isopropylpyrazolo[1,5-a]pyrimidin-6-yl)urea;
1-(5-chloro-6-(4-methyl-2H-1,2,3-triazol-2-yl)pyridin-3-yl)-3-(2-chloro-7-isopropylpyrazolo[1,5-a]pyrimidin-6-yl)urea;
1-(4-(2-aminopyrimidin-4-yl)-3-chlorophenyl)-3-(2-chloro-7-isopropylpyrazolo[1,5-a]pyrimidin-6-yl)urea;
1-(5-chloro-1-methyl-6-oxo-2-(1H-pyrazol-1-yl)-1,6-dihydropyridin-3-yl)-3-(2-chloro-7-isopropylpyrazolo[1,5-a]pyrimidin-6-yl)urea;
1-(5-chloro-6-ethoxypyridin-3-yl)-3-(2-chloro-7-isopropylpyrazolo[1,5-a]pyrimidin-6-yl)urea;
1-(5-bromopyridin-3-yl)-3-(2-chloro-7-isopropylpyrazolo[1,5-a]pyrimidin-6-yl)urea;
1-(2-chloro-7-isopropylpyrazolo[1,5-a]pyrimidin-6-yl)-3-(6-(1,1-dioxidoisothiazolidin-2-yl)-5-(trifluoromethyl)pyridin-3-yl)urea;
1-(3-chloro-4-(3-(hydroxymethyl)-5-methyl-1H-pyrazol-1-yl)phenyl)-3-(2-chloro-7-iso-propyl pyrazolo[1,5-a]pyrimidin-6-yl)urea;
1-(5-chloro-6-(1-methyl-1H-pyrazol-5-yl)pyridin-3-yl)-3-(2-chloro-7-isopropylpyrazolo[1,5-a]pyrimidin-6-yl)urea;
1-(2-chloro-7-isopropylpyrazolo[1,5-a]pyrimidin-6-yl)-3-(3,5-dichloro-4-(2H-1,2,3-triazol-2-yl)phenyl)urea;
1-(5-chloro-2-oxoindolin-7-yl)-3-(2-chloro-7-isopropylpyrazoio[1ᵢ5-a]pyrimidin-6-yl)urea;
1-(2-chloro-7-isopropylpyrazolo[1,5-a]pyrimidin-6-yl)-3-(1-methyl-2-oxo-5-(trifluoro-methyl)-1,2-dihydropyridin-3-yl)urea;
1-(5-chloro-2-((1-methylpyrrolidin-3-yl)oxy)-6-(2H-1,2,3-triazol-2-yl)pyridin-3-yl)-3-(2-chloro-7-isopropylpyrazolo[1,5-a]pyrimidin-6-yl)urea;
1-(7-(tert-butyl)-2-chloropyrazolo[1,5-a]pyrimidin-6-yl)-3-(5-chloro-6-(2H-1,2,3-triazol-2-yl)pyridin-3-yl)urea;
1-(7-(sec-butyl)-2-chloropyrazolo[1,5-a]pyrimidin-6-yl)-3-(5-chloro-6-(2H-1,2,3-triazol-2-yl)pyridin-3-yl)urea;
1-(2-chloro-7-(2-methyltetrahydrofuran-2-yl)pyrazolo[1,5-a]pyrimidin-6-yl)-3-(2-(trifluoro-methyl)pyridin-4-yl)urea;
(R)-1-(2-chloro-7-(1-methoxy-2-methylpropyl)pyrazolo[1,5-a]pyrimidin-6-yl)-3-(2-(trifluoro-methyl)pyridin-4-yl)urea;
1-(5-chloro-6-(2H-1,2,3-triazol-2-yl)pyridin-3-yl)-3-(2-chloro-7-cyclobutylpyrazolo[1,5-a]-pyrimidin-6-yl)urea;
(S)-1-(5-chloro-6-(2H-1,2,3-triazol-2-yl)pyridin-3-yl)-3-(2-chloro-7-(1-(2-methoxy-ethoxy)-ethyl)pyrazolo[1,5-a]pyrimidin-6-yl)urea;
(S)-1-(5-chloro-6-methoxypyridin-3-yl)-3-(2-chloro-7-(1-(2-methoxyethoxy)-ethyl)-pyrazolo-[1,5-a]pyrimidin-6-yl)urea;
(S)-1-(2-chloro-7-(1-(2-methoxyethoxy)ethyl)pyrazolo[1,5-a]pyrimidin-6-yl)-3-(2-(trifluoromethyl)-pyridin-4-yl)urea;
(R)-1-(5-chloro-6-(2H-1,2,3-triazol-2-yl)pyridin-3-yl)-3-(2-chloro-7-(1-(2-methoxy-ethoxy)-ethyl)pyrazolo[1,5-a]pyrimidin-6-yl)urea;
1-(5-chloro-6-(2H-1,2,3-triazol-2-yl)pyridin-3-yl)-3-(2-chloro-7-(1,4-dioxan-2-yl)-pyrazolo[1,5-a]pyrimidin-6-yl)urea;
(S)-1-(5-chloro-6-(2H-1,2,3-triazol-2-yl)pyridin-3-yl)-3-(2-chloro-7-(1-methoxypropan-2-yl)-pyrazolo[1,5-a]pyrimidin-6-yl)urea;
(R)-1-(5-chloro-8-(2H-1,2,3 triazol-2-yl)pyridin-3-yl)-3-(2-chloro-7-(1-methoxypropan-2-yl)-pyrazolo[1,5-a]pyrimidin-6-yl)urea;
(R)-1-(5-chloro-6-(2H-1,2,3-triazol-2-yl)pyridin-3-yl)-3-(2-chloro-7-(1-methoxyethyl)-pyrazolo[1,5-a]pyrimidin-6-yl)urea;
(R)-1-(2-chloro-7-(1-methoxyethyl)pyrazolo[1,5-a]pyrimidin-6-yl)-3-(2-(trifluoromethyl)-pyridin-4-yl)urea;
(R)-1-(2-chloro-7-(1-methoxyethyl)pyrazolo[1,5-a]pyrimidin-6-yl)-3-(1-methyl-2-oxo-5-(trifluoromethyl)-1,2-dihydropyridin-3-yl)urea;
1-(5-chloro-6-(2H-1,2,3-triazol-2-yl)pyridin-3-yl)-3-(2-chloro-7-(methoxy(phenyl)methyl)-pyrazolo[1,5-a]pyrimidin-6-yl)urea;
1-(5-chloro-6-(2H-1,2,3-triazol-2-yl)pyridin-3-yl)-3-(2-chloro-7-(1-(methoxymethyl) cyclobutyl)pyrazolo[1,5-a]pyrimidin-6-yl)urea;
1-(2-chloro-7-(1-(methoxymethyl)cyclobutyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-cyclopropylpyrazolo[1,5-a]pyrimidin-6-yl)urea;
1-(5-chloro-6-(2H-1,2,3-triazol-2-yl)pyridin-3-yl)-3-(2-chloro-7-(2-methoxyphenyl)pyrazolo[1,5-a]pyrimidin-6-yl)urea;
1-(6-(2H-1,2,3-triazoj-2-yl)-5-(trifluoromethyl)pyridine-3-yl)-3-(2-chloro-7-(tetrahydrofuran-2-yl)pyrazolo[1,5-a]pyrimidin-6-yl)urea;
1-(5-chloro-6-(2H-1,2,3-triazol-2-yl)pyridin-3-yl)-3-(2-chloro-7-(4-methyltetrahydro-2H-pyran-4-yl)pyrazolo[1,5-a]pyrimidin-6-yl)urea;
1-(5-chloro-6-(2H-1,2,3-triazol-2-yl)pyridin-3-yl)-3-(2-chloro-7-(1,2-dimethoxyethyl) pyrazolo[1,5-a]pyrimidin-6-yl)urea;
1-(5-chloro-6-(2H-1,2,3-triazol-2-yl)pyridin-3-yl)-3-(2-chloro-7-(tetrahydrofuran-3-yl) pyrazolo[1,5-a]pyrimidin-6-yl)urea;
1-(5-chloro-6-(2H-1,2,3-triazol-2-yl)pyridin-3-yl)-3-(2-chloro-7-(tetrahydro-2H-pyran-4-yl) pyrazolo[1,5-a]pyrimidin-6-yl)urea;
1-(5-chloro-6-(2H-1,2,3-triazol-2-yl)pyridin-3-yl)-3-(2-chloro-7-(methoxymethyl) pyrazolo[1,5-a]pyrimidin-6-yl)urea;
1-(5-chloro-6-(2H-1,2,3-triazol-2-yl)pyridin-3-yl)-3-(2-chloro-7-((tetrahydro-2H-pyran-4-yl)methyl)pyrazolo[1,5-a]pyrimidin-6-yl)urea;
1-(5-chloro-6-(2H-1,2,3-triazol-2-yl)pyridin-3-yl)-3-(2-chloro-7-(isopropoxymethyl) pyrazolo[1,5-a]pyrimidin-6-yl)urea;
1-(5-chloro-6-(2H-1,2,3-triazol-2-yl)pyridin-3-yl)-3-(2-chloro-7-methylpyrazolo[1,5-a]pyrimidin-6-yl)urea;
1-(6-(2H-1,2,3-triazol-2-yl)-5-(trifluoromethyl)pyridin-3-yl)-3-(2-chloro-7-(furan-2-yl) pyrazolo[1,5-a]pyrimidin-6-yl)urea;
1-(5-chloro-6-(2H-1,2,3-triazol-2-yl)pyridin-3-yl)-3-(2-chloro-7-(1,3-dimethoxypropyl) pyrazolo[1,5-a]pyrimidin-6-yl)urea;
(R)-1-(6-(2H-1,2,3-triazol-2-yl)-5-(trifluoromethyl)pyridin-3-yl)-3-(7-(1-(benzyloxy)ethyl)-2-chloropyrazolo[1,5-a]pyrimidin-6-yl)urea;
tert-butyl 2-(2-chloro-6-(3-(5-chloro-6-(2H-1,2,3-triazol-2-yl)pyridin-3-yl)ureido)pyrazolo[1,5-a]pyrimidin-7-yl)morpholine-4-carboxylate;
1-(7-(3-oxabicyclo[3.1.0]hexan-6-yl)-2-chloropyrazolo[1,5-a]pyrimidin-6-yl)-3-(5-chloro-6-methoxypyridin-3-yl)urea;
1-(5-chloro-6-(2H-1,2,3-triazol-2-yl)pyridin-3-yl)-3-(2-chloro-7-(5-oxaspiro[2.4]heptan-1-yl) pyrazolo[1,5-a]pyrimidin-6-yl)urea;
1-(5-chloro-6-methoxypyridin-3-yl)-3-(2-chloro-7-(pyridin-4-yl)pyrazolo[1,5-a]pyrimidin-6-yl)urea;
2-chloro-6-(3-(5-chloro-6-(2H-1,2,3-triazol-2-yl)pyridin-3-yl)ureido)-N,N-dimethyl pyrazolo[1,5-a]pyrimidine-7-carboxamide;
(S)-1-(5-chloro-6-(2H-1,2,3-triazol-2-yl)pyridin-3-yl)-3-(7-(1-methoxyethyl)-2-methy pyrazolo[1,5-a]pyrimidin-6-yl)urea;
1-(5-chloro-6-(2H-1,2,3-triazol-2-yl)pyridin-3-yl)-3-(2-methyl-7-(1-methylcyclopropyl) pyrazolo[1,5-a]pyrimidin-6-yl)urea;
1-(5-chloro-6-methoxypyridin-3-yl)-3-(2-methyl-7-(1-methylcyclopropyl)pyrazolo[1,5-a]pyrimidin-6-yl)urea;
(S)-1-(5-chloro-6-(2H-1,2,3-triazol-2-yl)pyridin-3-yl)-3-(2-chloro-7-(1-methoxy-2-methylpropyl)pyrazolo[1,5-a]pyrimidin-6-yl)urea;
(S)-1-(2-chloro-7-(1-methoxy-2-methylpropyl)pyrazolo[1,5-a]pyrimidin-6-yl)-3-(2-(trifluoromethyl)pyridin-4-yl)urea;
1-(5-chloro-6-(2H-1,2ₗ3-triazol-2-yl)pyridin-3-yl)-3-(2-chloro-7-(1-(methoxymethyl) cyclopropyl)pyrazolo[1,5-a]pyrimidin-6-yl)urea;
1-(2-chloro-7-(1-(methoxymethyl)cyclopropyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(7-(1-(methoxymethyl)cyclopropyl)-2-methylpyrazolo[1,5-a]pyrimidin-6-yl)urea;
1-(7-(1-(methoxymethyl)cyclopropyl)-2-methylpyrazolo[1,5-a]pyrimidin-6-yl)-3-(2-(trifluoromethyl)pyridin-4-yl)urea;
1-(2-chloro-7-(2-(tetrahydro-2H-pyran-4-yl)propan-2-yl)pyrazolo[1 !5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-(1,2-dimethoxypropan-2-yl)pyrazolo[1,5-a]pyrimidin-6-yl)urea;
(S)-1-(5-chloro-6-(2H-1,2,3-triazol-2-yl)pyridin-3-yl)-3-(2-chloro-7-(1-(2-(dimethylamino) ethoxy)ethyl)pyrazolo[1,5-a]pyrimidin-6-yl)urea;
1-(5-chloro-6-(2H-1,2,3-triazol-2-yl)pyridin-3-yl)-3-(2-chloro-7-((4-methylmorpholin-3-yl) methyl)pyrazolo[1,5-a]pyrimidin-6-yl)urea;
(S)-1-(5-chloro-6-(2H-1,2_{&gt;}3-triazol-2-yl)pyridin-3-yl)-3-(2-chloro-7-(1-methylpiperidin-2-yl) pyrazolo[1,5-a]pyrimidin-6-yl)urea;
1-(5-chloro-6-(2H-1,2,3-triazol-2-yl)pyridin-3-yl)-3-(2-chloro-7-((S)-1-((R)-2-methoxy-propoxy)ethyl)pyrazolo[1,5-a]pyrimidin-6-yl)urea;
1-(5-chloro-6-(2H-1,2,3-triazol-2-yl)pyridin-3-yl)-3-(2-chloro-7-(1-methyl-1H-imidazol-2-yl)pyrazolo[1,5-a]pyrimidin-6-yl)urea;
1-(5-chloro-6-methoxypyridin-3-yl)-3-(2-chloro-7-(5-methyltetrahydrofuran-2-yl)pyrazolo[1,5-a]pyrimidin-6-yl)urea;
1-(5-chloro-6-(2H-1,2,3-triazol-2-yl)pyridin-3-yl)-3-(2-chloro-7-(1-(dimethylamino) cyclopropyl) pyrazolo[1,5-a]pyrimidin-6-yi)urea;
(S)-1-(2-chloro-7-(1-methoxyethyl)pyrazolo[1,5-a]pyrimidin-6-yl)-3-(5-(difluoromethyl)-6-(2H-1_{!}2,3-triazol-2-yl)pyridin-3-yl)urea;
1-(2-chloro-7-(methoxy(tetrahydro-2H-pyran-4-yl)methyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-(2-(methoxymethyl) tetrahydrofuran-2-yl)pyrazolo[1,5-a]pyrimidin-6-yl)urea;
(S)-1-(2-chloro-7-(1-methoxyethyl)pyrazolo[1,5-a]pyrimidin-6-yl)-3-(2-(difluoromethyl) pyridin-4-yl)urea;
(S)-1-(5-chloro-6-(difluoromethoxy)pyridin-3-yl)-3-(2-chloro-7-(1-methoxyethyl) pyrazolo[1,5-a]pyrimidin-6-yl)urea;
(S)-1-(6-(2H-1,2,3-triazol-2-yl)-5-(trifluoromethyl)pyridin-3-yl)-3-(2-fluoro-7-(1-methoxy-ethyl)pyrazolo[1,5-a]pyrimidin-6-yl)urea;
(S)-1-(5-chloro-6-(2H-1,2,3-triazol-2-yl)pyridin-3-yl)-3-(2-fluoro-7-(1-methoxyethyl) pyrazolo[1,5-a]pyrimidin-6-yl)urea;
(S)-1-(5-chloro-6-(2H-1,2,3-triazol-2-yl)pyridin-3-yl)-3-(2-cyano-7-(1-methoxy-ethyl) pyrazolo[1,5-a]pyrimidin-6-yl)urea;
(S)-1-(5-chloro-6-(diiluoromethoxy)pyridin-3-yl)-3-(2-chloro-7-(1-(2-methoxyethoxy) ethyl)pyrazolo[1,5-a]pyrimidin-6-yl)urea;
1-(5-chloro-6-(2H-1,2,3-triazol-2-yl)pyridin-3-yi)-3-(2-chloro-7-((1R,2R)-1,2-propyl) pyrazolo[1,5-a]pyrimidin-6-yl)urea;
(S)-1-(5-chloro-2-(2-(dimethylamino)ethoxy)pyridin-3-yl)-3-(2-chloro-7-(1-methoxy ethyl)pyrazolo[1,5-a]pyrimidin-6-yl)urea;
1-(5-chloro-6-(2H-1,2,3-triazo!-2-yl)pyridin-3-yl)-3-(2-chloro-7-((S)-1-(((furan-3-yl)oxy)ethyl)pyrazolo[1,5-a]pyrimidin-6-yl)urea;
1-(5-chloro-6-(2H-1,2,3-triazol-2-yl)pyridin-3-yl)-3-(2-chloro-7-((S)-1-(((S)-tetrahydro-furan-3-yl)oxy)ethyl)pyrazolo[1,5-a]pyrimidin-6-yl)urea;
1-(5-chloro-6-(2H-1,2,3 triazol-2-yl)pyridin-3-yi)-3-(2-chloro-7-((S)-1-(((S)-ietrahydro-furan-3-yl)methoxy)ethyl)pyrazolo[1,5-a]pyrimidin-6-yl)urea;
1-(5-chloro-6-(2H-1,2,3-triazol-2-yl)pyridin-3-yl)-3-(2-chloro-7-((S)-1-(((R)-tetrahydro-furan-3-yl)methoxy)ethyl)pyrazolo[1,5-a]pyrimidin-6-yl)urea;
1-(5-chloro-6-(2H-1,2,3-triazol-2-yl)pyridin-3-yl)-3-(2-methyl-7-(2-methyltetrahydrofuran-2-yl)pyrazolo[1,5-a]pyrimidin-6-yl)urea;
(S)-1-(2-chloro-7-(1-methoxyethyl)pyrazolo[1,5-a]pyrimidin-6-yl)-3-(2-cyanopyridin-4-yl)urea;
1-(2-chloro-7-(2-(methoxymethyl)tetrahydrofuran-2-yl)pyrazolo[1,5-a]pyrimidin-6-yl)-3-(5-(trifluoromethyl)pyridin-3-yl)urea;
1-(6-(2H-1,2,3-triazol-2-yl)-5-(trifluoromethyl)pyridin-3-yl)-3-(2-chloro-7-(1-(dimethyl-amino)cydopropyl)pyrazolo[1,5-a]pyrimidin-6-yl)urea;
1-(6-(2H-1,2,3-triazol-2-yl)-5-(trifluoromethyl)pyridin-3-yl)-3-(2-chloro-7-(1,2-dimethoxy-ethyl)pyrazolo[1,5-a]pyrimidin-6-yl)urea;
1-(2-chloro-7-(1,2-dimethoxyethyl)pyrazolo[1,5-a]pyrimidin-6-yl)-3-(2-(trifluoromethyl) pyridin-4-yl)urea;
(S)-1-(6-(2H-1,2,3-triazol-2-yl)-5-(trifluoromethyl)pyridin-3-yl)-3-(2-chloro-7-(1-methoxy-propan-2-yl)pyrazolo[1,5-a]pyrimidin-6-yl)urea;
(R)-1-(6-(2H-1,2,3-triazol-2-yl)-5-(trifluoromethyl)pyridin-3-yl)-3-(2-chloro-7-(1-methoxy-propan-2-yl)pyrazolo[1,5-a]pyrimidin-6-yl)urea;
1-(5-(2H-1,2,3-triazol-2-yl)-2-(trifluoromethyl)pyridin-4-yl)-3-(2-chloro-7 [1,5-a]pyrimidin-6-yl)urea;
(R)-1-(6-(2H-1,2,3-triazo-2-yl)-5-(trifluoromethyl)pyridyin-3-yl)-3-(2-chloro-7-(1-hydroxy-ethyl) pyrazolo[1,5-a]pyrimidin-6-yl)urea;
(S)-1-(2-chloro-7-(1-hydroxyethyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-(morpholin-2-yl)pyrazolo[1,5-a]pyrimidin-8-yl)urea;
1-(5-chloro-6-(2H-1,2,3-triazol-2-yl)pyridin-3-yl)-3-(2-chloro-7-(4-methylmorpholin-2-yl) pyrazolo[1,5-a]pyrimidin-6-yl)urea;
1-(4-(4-(aminomethyl)-1H-pyrazol-1-yl)-3-chlorophenyl)-3-(2-chloro-7-isopropyl pyrazolo[1,5-a]pyrimidin-6-yl)urea;
1-(6-(4-(aminomethyl)-1H-pyrazol-1-yl)-5-chloropyridin-3-yl)-3-(2-chloro-7-isopropyl pyrazolo[1,5-a]pyrimidin-6-yl)urea;
(S)-1-(5-chloro-6-(2H-1,2,3-triazol-2-yl)pyridin-3-yl)-3-(2-chloro-7-(1-(methylamino) ethyl) pyrazolo[1,5-a]pyrimidin-6-yl)urea;
2-(3-(2-chloro-7-isopropylpyrazolo[1,5-a]pyrimidin-6-yl)ureido)-4-(trifluoromethyl)pyridine 1-oxide;
(R)-1-(5-chloro-6-(2H-1,2,3-triazol-2-yl)pyridin-3-yl)-3-(2-chloro-7-(1-(dimethylamino) ethyl)pyrazolo[1,5-a]pyrimidin-6-yl)urea;
(S)-1-(5-chloro-6-(2H-1_{!}2,3-triazol-2-yl)pyridin-3-yl)-3-(2-chloro-7-(1-(dimethylamino) ethyl)pyrazolo[1,5-a]pyrimidin-6-yl)urea;
1-(5-chloro-6-(2H-1,2,3-triazol-2-yl)pyridin-3-yl)-3-(2-chloro-7-(morpholin-3-yl) pyrazolo[1,5-a]pyrimidin-6-yl)urea;
(S)-1-(5-chloro-6-(2H-1,2,3-triazol-2-yl)pyridin-3-yl)-3-(2-chloro-7-(2-methyl-1-(methyl-amino) propyl)pyrazolo[1,5-a]pyrimidin-6-yl)urea;
1-(5-chloro-6-(2H-1,2,3-triazol-2-yl)pyridin-3-yl)-3-(2-chloro-7-(4-methylmorpholin-3-yl) pyrazolo[1,5-a]pyrimidin-6-yl)urea;
(R)-1-(5-chloro-6-(2H-1,2,3-triazol-2-yl)pyridin-3-yl)-3-(2-chloro-7-(1,2-dimethoxyethyl) pyrazolo[1,5-a]pyrimidin-6-yl)urea; and
(S)-1-(5-chloro-6-(2H-1,2,3-triazol-2-yl)pyridin-3-yl)-3-(2-chloro-7-(1,2-dimethoxyethyl) pyrazolo[1,5-a]pyrimidin-6-yl)urea.

17. The method according to claim 1, wherein said MALT1 inhibitor is a small molecule of the formula (II) or a pharmaceutically acceptable salt thereof, 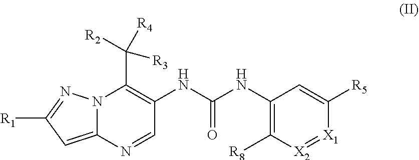
wherein
R₁ is fluoro, chloro, methyl or cyano;
R₂ and R₃ are independently from each other C₁-C₆ alkoxy optionally substituted by C₁-C₆ alkoxy; C₁-C₈, alkyl optionally substituted by halogen or C₁-C₆ alkoxy; amino optionally substituted by C₁-C₆ alkyl; phthalimido; or hydroxy optionally substituted by a 5 or 6 membered heterocyclic ring comprising a nitrogen or oxygen heteroatom wherein said ring is optionally substituted by C₁-C₃ alkyl carbonyl;
or R₂ and R₃ together with carbon atom to which they are attached form a 3-5 membered carbocyclic ring or heterocyclic ring comprising 1 heteroatom selected from N and O;
R₄ is hydrogen; C₁-C₆ alkyl optionally substituted by C₁-C₆ alkoxy;
X₁ is N, N—O or CR₆;
X₂ is N or CR₇;
R₅ is chloro; cyano; or C₁-C₆ alkyl optionally substituted by halogen and/or hydroxy;
R₆ is hydrogen; oxo; methoxy; 1,2,3-triazole-2-yl; or aminocarbonyl substituted at the nitrogen atom by R₉ and R₁₀;
R₇ is hydrogen; C₁-C₆ alkyl optionally substituted by halogen and/or hydroxy; or N, N-dimethylaminocarbonyl;
R₈ is hydrogen; C₁-C₆ alkoxy optionally substituted by methoxy or amino;
R₉ and R₁₀ are independently of each other hydrogen; C₁-C₆ alkyl optionally substituted by C₁-C₆ alkoxy, N-mono-C₁-C₆ alkyl amino, or N, N-di-C₁-C₆ alkyl amino; or
R₉ and R₁₀ together with the nitrogen atom to which they are attached form a 5-7 membered heterocyclic ring having one, two or three ring hetero atoms selected from the group consisting of oxygen, nitrogen and sulphur, that ring being optionally substituted by C₁-C₆ alkyl, hydroxy or oxo;
with the proviso that X₁ and X₂ must not be N at the same time, or X₁ must not be N-O when X₂ is N.

18. The method according to claim 17, wherein
R₁ is fluoro or chloro;
R₂ is C₁-C₆ alkyl optionally substituted by C₁-C₆ alkoxy;
R₃ is C₁-C₆ alkoxy optionally be substituted by C₁-C₆ alkoxy;
R₄ is hydrogen;
X₁ is N;
X₂ is CR₇;
R₅ is chloro; cyano; difluoromethyl; trifluoromethyl;
R₇ is hydrogen; and
R₈ is hydrogen.

19. The method according to claim 17, wherein
R₁ is fluoro or chloro;
R₂ is C₁-C₆ alkyl optionally substituted by C₁-C₆ alkoxy;
R₃ is C₁-C₆ alkoxy optionally be substituted by C₁-C₆ alkoxy;
R₄ is hydrogen;
X₁ is CR₆
X₂ is N;
R₅ is chloro; cyano; difluoromethyl; trifluoromethyl;
R₆ is hydrogen; oxo; methoxy; 1,2,3-triazole-2-yl; N-methylaminocarbonyl, N,N-dimethylaminocarbonyl; pyrrolidin-1-yl carbonyl and
R₈ is hydrogen.

20. The method according to claim 17, wherein
R₁ is methyl, fluoro or chloro;
R₂ is C₁-C₆ alkyl;
R₃ is C₁-C₆ alkoxy;
R₄ is hydrogen;
X₁ is CR₆
X₂ is N;
R₅ is chloro; cyano; difluoromethyl; trifluoromethyl;
R₆ is hydrogen; methoxy; 1,2,3-triazole-2-yl; N-methylaminocarbonyl, N,N-dimethylamino carbonyl; pyrrolidin-1-yl carbonyl and
R₈ is hydrogen.

21. The method according to claim 17, wherein
R₁ is methyl, fluoro or chloro;
R₂ is C₁-C₆ alkyl;
R₃ is C₁-C₆ alkoxy;
R₄ is hydrogen;
X₁-is N;
X₂ is CR₇;
R₅ is chloro; cyano; difluoromethyl; trifluoromethyl;
R₇ is hydrogen; and
R₈ is hydrogen.

22. The method according to claim 17, wherein
R₁ is fluoro or chloro;
R₂ is C₁-C₆ alkoxy;
R₃ is C₁-C₆ alkyl;
R₄ is hydrogen;
X₁ is CR₆
X₂ is N;
R₅ is chloro; cyano; difluoromethyl; trifluoromethyl;
R₆ is hydrogen; methoxy; 1,2,3-triazole-2-yl; N-methylaminocarbonyl, N,N-imethylamino carbonyl; pyrrolidin-1-yl carbonyl; and
R₈ is hydrogen.

23. The method according to claim 17, wherein
R₁ is fluoro or chloro;
R₂ is C₁-C₆ alkoxy;
R₃ is C₁-C₆ alkyl;
R₄ is hydrogen;
X₁ is N;
X₂ is CR₇;
R₅ is chloro; cyano; difluoromethyl; trifluoromethyl;
R₇ is hydrogen; and
R₈ is hydrogen.

24. The method according to claim 17, wherein the MALT1 inhibitor is selected from
(S)-1-(5-cyanopyridin-3-yl)-3-(7-(1-methoxyethyl)-2-methylpyrazolo[1,5-a]pyrimidin-6-yl)urea;
(S)-1-(2-(difluoromethyl)pyridin-4-yl)-3-(2-fluoro-7-(1-methoxyethyl) pyrazolo[1,5-a]pyrimidin-6-yl)urea;
(S)-1-(2-chloro-7-(1-methoxyethyl)pyrazolo[1,5-a]pyrimidin-6-yl)-3-(2-(trifluoromethyl)pyridin-4-yl)urea;
1-(5-chloro-6-(2H-1,2,3-triazol-2-yl)pyridin-3-yl)-3-(2-chloro-7-isopropylpyrazolo[1,5-a]pyrimidin-6-yl)urea;
(S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea;
(S)-1-(5-cyano-6-methoxypyridin-3-yl)-3-(2-fluoro-7-(1-methoxyethyl) pyrazolo[1,5-a]pyrimidin-6-yl)urea;
(S)-1-(6-(2H-1,2,3-triazol-2-yl)-5-(trifluoromethyl)pyridin-3-yl)-3-(2-chloro-7-(1-(2-methoxyethoxy)ethyl)pyrazolo[1,5-a]pyrimidin-6-yl)urea;
(S)-1-(6-(2H-1,2,3-triazol-2-yl)-5-(trifluoromethyl)pyridin-3-yl)-3-(2-chloro-7-(1-methoxy-2-methyl-propyl)pyrazolo[1,5-a]pyrimidin-6-yl)urea;
1-(2-chloro-7-(1-(methoxymethyl)cyclopropyl)pyrazolo[1,5-a]pyrimidin-6-yl)-3-(5-cyanopyridin-3-yl)urea;
1-(5-chloro-6-(2H-1,2,3-triazol-2-yl)pyridin-3-yl)-3-(2-chloro-7-((1R,2S)-1,2-dimethoxypropyl) pyrazolo[1,5-a]pyrimidin-6-yl)urea;
(S)-1-(2-chloro-7-(1-methoxyethyl)pyrazolo[1,5-a]pyrimidin-6-yl)-3-(5-cyano-6-(2H-1,2,3-triazol-2-yl)pyridin-3-yl)urea;
(S)-1-(5-cyanopyridin-3-yl)-3-(2-fluoro-7-(1-methoxyethyl)pyrazolo[1,5-a]pyrimidin-6-yl)urea;
1-(7-((S)-1-(((R)-1-acetylpyrrolidin-3-yl)oxy)ethyl)-2-chloropyrazolo[1,5-a]pyrimidin-6-yl)-3-(5-chloro-6-(2H-1,2,3-triazol-2-yl)pyridin-3-yl)urea;
(S)-1-(5-chloro-6-(2H-1,2,3-triazol-2-yl)pyridin-3-yl)-3-(2-fluoro-7-(1-methoxy-2-methylpropyl)-pyrazolo[1,5-a]pyrimidin-6-yl)urea;
(S)-1-(5-cyano-6-(2H-1,2,3-triazol-2-yl)pyridin-3-yl)-3-(7-(1-methoxy-2-methylpropyl)-2-methylpyrazolo[1,5-a]pyrimidin-6-yl)urea;
(S)-1-(2-chloro-7-(1-methoxyethyl)pyrazolo[1,5-a]pyrimidin-6-yl)-3-(5-cyano-6-methoxypyridin-3-yl)urea;
1-(2-fluoro-7-((S)-1-methoxyethyl)pyrazolo[1,5-a]pyrimidin-6-yl)-3-(2-(1-hydroxyethyl)-6-(trifluoromethyl)pyridin-4-yl)urea;
(S)-1-(5-cyano-6-(2H-1,2,3-triazol-2-yl)pyridin-3-yl)-3-(2-fluoro-7-(1-methoxyethyl)pyrazolo[1,5-a]pyrimidin-6-yl)urea;
1-(2-chloro-7-(1,2-dimethoxyethyl)pyrazolo[1,5-a]pyrimidin-6-yl)-3-(5-cyano-6-(2H-1,2,3-triazol-2-yl)pyridin-3-yl)urea;
1-(2-chloro-7-((S)-1-methoxyethyl)pyrazolo[1,5-a]pyrimidin-6-yl)-3-(2-(2,2,2-trifluoro-1-hydroxy-ethyl)pyridin-4-yl)urea;
(S)-1-(5-chloro-2-(2-methoxyethoxy)pyridin-3-yl)-3-(2-chloro-7-(1-methoxyethyl)-pyrazolo[1,5-a]-pyrimidin-6-yl)urea;
(S)-1-(5-cyano-6-methoxypyridin-3-yl)-3-(7-(1-methoxy-2-methylpropyl)-2-methylpyrazolo[1,5-a]-pyrimidin-6-yl)urea;
(S)-1-(2-cyanopyridin-4-yl)-3-(2-fluoro-7-(1-methoxyethyl)pyrazolo[1,5-a]pyrimidin-6-yl)urea;
(S)-1-(5-cyano-6-methoxypyridin-3-yl)-3-(7-(1-methoxyethyl)-2-methylpyrazolo[1,5-a]pyrimidin-6-yl)urea;
1-(2-chloro-7-((1R,2S)-1,2-dimethoxypropyl)pyrazolo[1,5-a]pyrimidin-6-yl)-3-(5-cyano-6-(2H-1,2,3-triazol-2-yl)pyridin-3-yl)urea;
1-(7-((S)-1-(((S)-1-acetylpyrrolidin-3-yl)oxy)ethyl)-2-chloropyrazolo[1,5-a]pyrimidin-6-yl)-3-(5-cyano-6-methoxypyridin-3-yl)urea;
(S)-1-(5-cyano-6-(2H-1,2,3-triazol-2-yl)pyridin-3-yl)-3-(7-(1-methoxyethyl)-2-methylpyrazolo[1,5-a]pyrimidin-6-yl)urea;
(S)-6-chloro-4-(3-(2-chloro-7-(1-methoxyethyl)pyrazolo[1,5-a]pyrimidin-6-yl)ureido)-N,N-dimethylpicolinamide;
(S)-1-(5-(difluoro-methyl)pyridin-3-yl)-3-(2-fluoro-7-(1-methoxyethyl)-pyrazolo[1,5-a]pyrimidin-6-yl)urea;
(S)-1-(2-fluoro-7-(1-methoxyethyl)pyrazolo[1,5-a]pyrimidin-6-yl)-3-(5-(trifluoro-methyl)pyridin-3-yl)urea;
(S)-3-chloro-5-(3-(2-chloro-7-(1-methoxyethyl)pyrazolo[1,5-a]pyrimidin-6-yl)ureido)-N,N-dimethylpicolinamide;
(S)-1-(5-chloro-pyridin-3-yl)-3-(2-fluoro-7-(1-methoxyethyl)pyrazolo[1,5-a]pyrimidin-6-yl)urea;
(S)-1-(5-chloro-6-(pyrrolidine-1-carbonyl)pyridin-3-yl)-3-(2-chloro-7-(1-methoxyethyl)pyrazolo-[1,5-a]pyrimidin-6-yl)urea
(S)-3-chloro-5-(3-(2-chloro-7-(1-methoxyethyl)pyrazolo[1,5-a]pyrimidin-6-yl)ureido)-N-methylpicolinamide
(S)-1-(2-chloro-7-(1-methoxyethyl)pyrazolo[1,5-a]pyrimidin-6-yl)-3-(5-chloropyridin-3-yl)urea;
(S)-1-(7-(1-aminoethyl)-2-chloropyrazolo[1,5-a]pyrimidin-6-yl)-3-(5-chloro-6-(2H-1,2,3-triazol-2-yl)pyridin-3-yl)urea;
(S)-1-(5-cyanopyridin-3-yl)-3-(7-(1-hydroxyethyl)-2-methylpyrazolo[1,5-a]pyrimidin-6-yl)urea;
(S)-1-(2-(difluoromethyl)pyridin-4-yl)-3-(2-fluoro-7-(1-hydroxyethyl) pyrazolo[1,5-a]pyrimidin-6-yl)urea;
1-(2-((S)-2-aminopropoxy)-5-chloropyridin-3-yl)-3-(2-chloro-7-((S)-1-methoxyethyl)pyrazolo[1,5-a]pyrimidin-6-yl)urea;
(S)-2-(difluoromethyl)-4-(3-(2-fluoro-7-(1-methoxyethyl)pyrazolo[1,5-a]pyrimidin-6-yl)ureido)pyridine 1-oxide;
1-(2-chloro-7-((1R,2S)-1,2-dimethoxypropyl)pyrazolo[1,5-a]pyrimidin-6-yl)-3-(5-cyano-6-methoxypyridin-3-yl)urea;
1-(2-chloro-7-(1-(methoxymethyl)cyclopropyl)pyrazolo[1,5-a]pyrimidin-6-yl)-3-(2-cyanopyridin-4-yl)urea; and
(S)-3-chloro-5-(3-(2-fluoro-7-(1-methoxyethyl)pyrazolo[1,5-a]pyrimidin-6-yl)ureido)picolinamide;
and pharmaceutically acceptable salts of any of the above.

25. The method according to claim 17, wherein X₁ is N and X₂ is not N, or X₁ is not N and X₂ is N.

26. The method according to claim 1, wherein the MALT1 inhibitor is a small molecule of the formula III 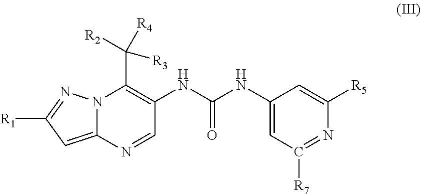
R₁ is fluoro or chloro;
R₂ and R₃ are independently from each other C₁-C₆ alkyl or C₁-C₆ alkoxy;
R₄ is hydrogen;
R₅ and R₇ are independently from each other hydrogen; cyano; halogen or C₁-C₆ alkyl optionally substituted by fluoro and/or hydroxyl.

27. The method according to claim 1, wherein the MALT1 inhibitor is a small molecule of the formula (IV) or a pharmaceutically acceptable salt thereof, wherein 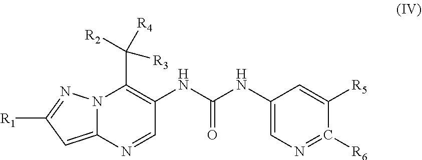
R₁ is fluoro or chloro;
R₂ and R₃ are independently from each other C₁-C₆ alkyl or C₁-C₆ alkoxy;
R₄ is hydrogen;
R₅ is hydrogen; cyano; halogen or C₁-C₆ alkyl optionally substituted by fluoro and/or hydroxyl; and
R₆ is hydrogen; 1,2,3-triazole-2-yl; N,N-dimethylaminocarbonyl; N-monomethylaminocarbonyl; or pyrrolidin-1-yl carbonyl.

28. The method according to claim 1, wherein the method further comprises the administration of at least one of:
(i) an additional immunomodulatory agent which blocks or inhibits an immune system checkpoint, which checkpoint may or may not be a component of the NFκB pathway; and/or
(ii) an agent which directly stimulates an immune effector response; and/or
(iii) a composition comprising a tumour antigen or immunogenic fragment thereof; and/or
(iv) a chemotherapeutic agent.

29. The method according to claim 28, wherein said additional immunomodulatory agent blocks or inhibits at least one of the following checkpoints:
a) The interaction between Indoleamine 2,3-dioxygenase (IDO1) and its substrate;
b) The interaction between PD1 and PDL1 and/or PD1 and PDL2;
c) The interaction between CTLA4 and CD86 and/or CTLA4 and CD80;
d) The interaction between B7-H3 and/or B7-H4 and their respective ligands;
e) The interaction between HVEM and BTLA;
f) The interaction between GAL9 and TIM3;
g) The interaction between MHC class I or II and LAG3;
h) The interaction between MHC class I or II and KIR;
i) The interaction between OX40(CD134) and OX40L (CD252);
k) The interaction between CD40 and CD40L (CD154);
l) The interaction between 4-1BB (CD137) and ligands including 4-1BBL;
m) The interaction between GITR and ligands including GITRL;
preferably wherein said checkpoint is (b) or (c) and wherein said agent is an antibody which binds to a component of the checkpoint.

30. The method according to claim 28, wherein said agent which directly stimulates an immune effector response is a cytokine or chemokine (or an agent which stimulates production of either), a tumour specific adoptively transferred T cell population, or an antibody specific for a protein expressed by a tumour cell, optionally wherein said agent is IFNα, IFNβ, IFNγ, IFNλ, IL-2, CXCL9, CXCL10, CXCL11, or Bacille Calmette-Guerin (BCG).

31. The method according to claim 28, wherein said composition comprising a tumour antigen or immunogenic fragment thereof is an autologous tumour cell vaccine.

32. The method according to claim 28, wherein said chemotherapeutic agent is mitomycin, valrubicin, docetaxel, thiotepa or gemcitabine.

33. The method according to claim 28 which is for the treatment of bladder cancer, wherein BCG and/or a chemotherapeutic agent selected from mitomycin, valrubicin, docetaxel, thiotepa and gemcitabine is administered intravesically to the subject.

34. The method according to claim 28 which is for the treatment of colon cancer, wherein BCG and/or an autologous tumour cell vaccine is administered intravesically to the subject, optionally as a single combined preparation.
